(19) United States
(12) Patent Application Publication
(10) Pub. No.: US 2026/0158073 A1
(43) Pub. Date: Jun. 11, 2026

(54) COMPOSITIONS AND METHODS FOR ASSESSING, TREATING, OR REDUCING AGING-RELATED FUNCTIONAL DECLINE

(71) Applicant: The Children's Medical Center Corporation, Boston, MA (US)

(72) Inventors: Yi Zhang, Boston, MA (US); Yuting Wang, Boston, MA (US); Xiangle Ren, Boston, MA (US)

(73) Assignee: The Children's Medical Center Corporation, Boston, MA (US)

(21) Appl. No.: 19/418,153

(22) Filed: Dec. 12, 2025

Related U.S. Application Data
(63) Continuation of application No. PCT/US2024/033619, filed on Jun. 12, 2024.
(60) Provisional application No. 63/472,759, filed on Jun. 13, 2023.

(51) Int. Cl. A61K 35/28; A61P 7/00; C07K 16/28; C12N 5/00; C12N 5/0789; G01N 33/50

(52) U.S. Cl. CPC A61K 35/28; A61P 7/00; C07K 16/2803; C12N 5/0087; C12N 5/0647; G01N 33/5094; G01N 2333/70503

(57) ABSTRACT

Described herein are compositions and methods for reducing, improving, abrogating, or alleviating age- or aging-related functional decline in an older or aging subject by reducing the ratio or number of hematopoietic stem cells (HSCs) expressing high levels of the glycoprotein CD150 (CD150^{high} HSCs), which may be dysfunctional or defective, in the subject. The methods involve reducing or removing dysfunctional or defective high CD150^{high} HSCs from a sample obtained from a subject. Methods for administering to an old, aged, or aging subject having CD150^{high} HSCs a composition comprising hematopoietic stem cells expressing low levels of the glycoprotein CD150 (CD150^{low}HSCs) or reducing or removing CD150^{high} HSCs with an antibody-drug conjugate provide therapeutics that rejuvenate the differentiated hematopoietic cell types in the blood as well as improve the physical health of the aging subject to counter functional decline that occurs in aging HSCs.

### CROSS REFERENCE TO RELATED APPLICATIONS

This application is a continuation under 35 U.S.C. § 111 (a) of PCT International Patent Application No. PCT/US2024/033619, filed Jun. 12, 2024, designating the United States and published in English, which claims priority to and benefit of U.S. Provisional Application No. 63/472,759, filed on Jun. 13, 2023, the entire contents of which are incorporated by reference herein.

### SEQUENCE LISTING

The instant application contains a Sequence Listing which has been submitted electronically as an XML file and is hereby incorporated by reference in its entirety. The Sequence Listing, created Jun. 24, 2024, is named “167705-033501PCT_SL.xml” and is 9,020 bytes in size.

### BACKGROUND

Aging is a process accompanied by functional decline in tissues and organs with great social and medical consequences. Previous studies have demonstrated that aged hematopoietic stem cells (HSCs) are functionally compromised, which at least partly contributes to aging-related decline of the overall health of the body. However, the underlying mechanism of aging is largely unknown. Needed in the art are compositions and methods for assessing, reducing, attenuating, mitigating, and/or abrogating the aging process. Such methods are important for the physical health and well-being of mammalian subjects, including humans, as described herein.

### SUMMARY

Developing effective anti-aging strategies is of great significance. As described and demonstrated herein, transplantation of young hematopoietic stem cells (HSCs) into old mice can mitigate aging phenotypes, underscoring the crucial role played by HSCs in the aging process. Through comprehensive molecular and functional analyses, a subset of HSCs in aged mice was identified that exhibit “youthful” molecular profiles and functions, marked by low expression levels of CD150 on HSCs (“CD150^{low} HSCs”). Mechanistically, CD150^{low} HSCs from old mice can effectively differentiate into downstream lineage cells, in contrast to their CD150^{high} counterparts. Notably, transplantation of old CD150^{low} HSCs attenuates aging phenotypes and prolongs lifespan of elderly mice compared to those transplanted with unselected or CD150^{high} HSCs. Reducing the dysfunctional CD150^{high} HSCs was able to alleviate aging phenotypes in old recipient mice. Thus, as described herein, the presence of “youthful” HSCs is demonstrated in old mice, and rejuvenation of physical health and improvements in the aging process can be achieved by removal of the dysfunctional HSCs.

Featured and described herein are compositions comprising selected hematopoietic stem cells (HSCs) having a reduction in levels of CD150 relative to a reference, and methods of using the same for administration, transplantation, or engraftment in a subject in need thereof. In an embodiment, the subject is an aging or aged subject. Compositions and methods for reducing and/or removing functionally defective HSCs are provided to reduce, abrogate, mitigate, and/or alleviate aging-related phenotypes in old or aged subjects, e.g., mammalian subjects, including humans.

Also featured and described herein are compositions and methods for ameliorating an old, aging, or aged phenotype, and/or rejuvenating old or aging mammalian subjects in need thereof, including human subjects, by targeted reduction, removal, depletion, or ablation of dysfunctional HSCs from a sample of the subject, such as a peripheral blood sample, bone marrow, or a tissue preparation, in vitro or ex vivo, or by removal, depletion, or ablation of dysfunctional HSCs from the subject in vivo.

In an aspect, a method for characterizing hematopoietic stem cell (HSC) functionality is provided, in which the method involves detecting the level of CD150 polypeptide or polynucleotide in an HSC, wherein an alteration in the level of CD150 relative to a reference is indicative of HSC functionality. In an embodiment of the method, a reduction in the level of the CD150 polypeptide or polynucleotide relative to the reference indicates that the HSC has increased self-renewal capacity and is capable of balanced differentiation.

In another aspect, a method for selecting a hematopoietic stem cell (HSC) for engraftment in a subject in need thereof is provided, in which the method involves detecting the level of CD150 polypeptide or polynucleotide expressed in an HSC, wherein detection of a reduced level of CD150 expression relative to a reference selects the HSC for engraftment in a subject. In an embodiment of the method, a reduction in the level of CD150 polypeptide or polynucleotide relative to the reference indicates that the HSC should be selected for engraftment. In an embodiment of the method, an increase in the level of CD150 polypeptide or polynucleotide relative to the reference indicates that the HSC should not be selected for engraftment.

In an embodiment of the methods of the above-delineated aspects and/or embodiments thereof, the HSC is present in a population marked by heterogeneity in HSC functionality. In an embodiment of the methods of the above-delineated aspects and/or embodiments thereof, the method further comprises detecting the expression of one or more markers selected from the group consisting of Sbspon, Ehd3, Clu, Dpy19l2, Kcnb2, Selp, B3gnt8, Jam2, Enpp5, Gpr183, and Ramp2, wherein expression of the one or more markers positively correlates with CD150 expression. In an embodiment of the methods of the above-delineated aspects and/or embodiments thereof, the method further comprises detecting the expression of one or more markers selected from the group consisting of Serpinb1a, Usp6nl, Lrr1, Col4a2, Gng2, Kif15, Rnase6, or Arhgap30, wherein expression of said markers negatively correlates with CD150 expression. In an embodiment of the methods of the above-delineated aspects and/or embodiments thereof, the method further comprises characterizing the level of one or more of 332 HSC aging marker genes (Tables 1A, 1B), wherein detecting an increase in one or more of said genes is indicative of HSC functional decline. In an embodiment of the methods of the above-delineated aspects and/or embodiments thereof, the method levels of CD150 polypeptide are detected using FACS analysis, and wherein levels of CD150 polynucleotide are detected using RNA-seq analysis.

In another aspect, a method of rejuvenating blood components of an aging or aged subject or alleviating aging-related hematopoietic cell functional decline is provided, in which the method involves administering to the subject an effective amount of hematopoietic stem cells (HSCs) expressing low levels of CD150 polypeptide (CD150^{low} HSCs) or a composition thereof comprising a physiologically acceptable carrier, excipient or diluent; wherein the CD150^{low} HSCs differentiate into hematopoietic cell types which rejuvenate blood components in the aged subject. In an embodiment of the method, CD150^{low} HSCs have a mean fluorescence intensity of less than about 4×10³ cell surface expressed CD150 proteins per cell based on flow cytometry or fluorescence activated cell sorting (FACS) analysis. In an embodiment of the method, the CD150^{low} HSCs comprise the lowest 25% subpopulation of a Long-Term-HSC population of bone marrow which expresses a Lin⁻Sca-1⁺c-Kit⁺CD48⁻CD34⁻CD150⁺ cell surface biomarker signature based on flow cytometry or fluorescence activated cell sorting (FACS) analysis. In an embodiment of the method, CD150^{low} HSCs express marker genes Rnase6 and Arhgap30.

In an embodiment of the above-delineated method of rejuvenating blood components of an aging or aged subject or alleviating aging-related hematopoietic cell functional decline, the administration is intravenous or is via transplant or engraftment. In an embodiment of the method, the cell types which differentiate from the CD150^{low} HSCs to rejuvenate the blood components comprise B lymphocytes, T lymphocytes, and myeloid cells; and/or wherein the rejuvenated blood components are in an activated state In an embodiment of the method, the aging or aged subject is selected by detecting hematopoietic stem cells expressing high levels of CD150 polypeptide (CD150^{high} HSCs) in a sample obtained from the subject. In an embodiment, the CD150^{high} HSCs have a mean fluorescence intensity of greater than about 4×10³ cell surface expressed CD150 proteins per cell based on flow cytometry or fluorescence activated cell sorting (FACS) analysis. In an embodiment, the CD150^{high} HSCs comprise the highest 25% subpopulation of a Long-Term-HSC population of bone marrow which expresses a Lin⁻Sca-1⁺c-Kit⁺CD48⁻CD34⁻CD150⁺ cell surface biomarker signature based on flow cytometry or fluorescence activated cell sorting (FACS) analysis. In an embodiment, the CD150^{high} HSCs express a CD150 mRNA level of between about 0.3 to about 2 RNA Units per cell. In an embodiment of the methods, the subject is greater than 50 years of age or is greater than 70 years of age. In embodiments of the method, the sample is a blood, plasma, serum, bone marrow, or tissue sample. In an embodiment of the method, following administration of the CD150^{low} HSCs, the subject exhibits increased numbers of naïve T cells and a decrease in central memory T cell (Tcm) and effector memory T cell (Tem) ratio in the CD8⁺ and CD4⁺ subgroups of T cells.

In another aspect, a method of improving physical health and aging-related hematopoietic cell functional decline of a selected subject is provided, in which the method involves selecting the subject by detecting hematopoietic stem cells (HSC) expressing high levels of CD150 polypeptide (CD150^{high} HSCs) in a sample obtained from the subject; and administering to the subject an effective amount of hematopoietic stem cells (HSCs) expressing low levels of CD150 polypeptide (CD150^{low} HSCs) or a composition thereof comprising a physiologically acceptable carrier, excipient or diluent; wherein the CD150^{low} HSCs differentiate into hematopoietic cell types which improve physical health and aging-related hematopoietic cell functional decline in the subject. In an embodiment of the method, the CD150^{high} HSCs have a mean fluorescence intensity of greater than about 4×10³ cell surface expressed CD150 proteins per cell based on flow cytometry or fluorescence activated cell sorting (FACS) analysis. In an embodiment of the method, the CD150^{high} HSCs comprise the highest 25% subpopulation of a Long-Term-HSC population of bone marrow which expresses a Lin⁻Sca-1⁺c-Kit⁺CD48⁻CD34⁻CD150⁺ cell surface biomarker signature in the sample based on flow cytometry or fluorescence activated cell sorting (FACS) analysis. In an embodiment of the method, the CD150^{high} HSCs express a CD150 mRNA level of between about 0.3 to about 2 RNA Units per cell. In an embodiment of the method and/or embodiments thereof, the CD150^{low} HSCs administered to the subject have a mean fluorescence intensity of less than about 4×10³ cell surface expressed CD150 proteins per cell based on flow cytometry or fluorescence activated cell sorting (FACS) analysis. of the method and/or embodiments thereof, the CD150^{low} HSCs administered to the subject comprise the lowest 25% subpopulation of a Long-Term-HSC population of bone marrow which expresses a Lin⁻Sca-1⁺c-Kit⁺CD48⁻CD34⁻CD150⁺ cell surface biomarker signature based on flow cytometry or fluorescence activated cell sorting (FACS) analysis. In an embodiment of the method and/or embodiments thereof, the CD150^{low} HSCs express marker genes Serpinb1a, Usp6nl, Lrr1, Col4a2, Gng2, Kif15, Rnase6, or Arhgap30. In an embodiment of the method and/or embodiments thereof, the CD150^{low} HSCs express marker genes Rnase6 and Arhgap30. In an embodiment of the method and/or embodiments thereof, the administration to the subject is intravenous or is via transplant or explant. In an embodiment of the method and/or embodiments thereof, improving physical health and improving aging-related hematopoietic cell functional decline of the subject comprises administering CD150^{low} HSCs which differentiate into activated or activatable B lymphocytes, T lymphocytes, and myeloid cells. In an embodiment of the method and/or embodiments thereof, the subject is greater than 50 years of age or is greater than 70 years of age. In embodiment of the method and/or embodiments thereof, the sample is a blood, plasma, serum, or bone marrow sample. In an embodiment of the method and/or embodiments thereof, following administration of the CD150^{low} HSCs, the subject exhibits increased numbers of naïve T cells and a decrease in central memory T cell (Tcm) and effector memory T cell (Tem) ratio in the CD8⁺ and CD4⁺ subgroups of T cells. In an embodiment of the method and/or embodiments thereof, the subject having CD150^{high} HSCs is selected by further detecting in cells of the sample one or more age-related genes selected from Sbspon, Ehd3, Clu, Dpy19l2, Kcnb2, Selp, B3gnt8, Jam2, Enpp5, Gpr183, and Ramp2.

In an embodiment of any one of the above-delineated methods and/or embodiments thereof, the CD150^{low} HSCs highly express one or more genes selected from Serpinb1a, Usp6nl, Lrr1, Col4a2, Gng2, Kif15, Rnase6, or Arhgap30. In an embodiment of any one of the above-delineated methods and/or embodiments thereof, the CD150^{low} HSCs administered to the subject dilute or reduce the numbers of CD150^{high} HSCs in the subject, thereby rejuvenating the blood with CD150^{low} HSCs capable of differentiation into activated or activatable hematopoietic cells and cell types. In an embodiment, the CD150^{low} HSCs administered to the subject increase the numbers of activated versus quiescent CD150-expressing HSCs in the subject.

In another aspect, a composition comprising an effective amount of hematopoietic stem cells expressing a low abundance of the CD150 glycoprotein (CD150^{low} HSCs) is provided, wherein said CD150^{low} HSCs have a mean fluorescence intensity of less than about 4×10³ cell surface expressed CD150 proteins per cell or wherein CD150^{low} HSCs comprise the lowest 25% subpopulation of a Long-Term-HSC population of bone marrow which expresses a Lin⁻Sca-1⁺c-Kit⁺CD48⁻CD34⁻CD150⁺ cell surface biomarker signature based on flow cytometry or fluorescence activated cell sorting (FACS) analysis. In an embodiment, the composition further includes a physiologically acceptable carrier, excipient or diluent. In an embodiment of the composition, the CD150^{low} HSCs express marker genes Rnase6 and Arhgap30. In an embodiment of the composition and/or embodiments thereof, CD150^{low} HSCs highly express one or more genes selected from Serpinb1a, Usp6nl, Lrr1, Col4a2, Gng2, Kif15, Rnase6, or Arhgap30. In an embodiment, the composition comprises a saline solution or a buffer solution.

In another aspect, the use of a composition comprising an effective amount of hematopoietic stem cells expressing a low abundance of the CD150 glycoprotein (CD150^{low} HSCs) is provided, wherein the CD150^{low} HSCs have a mean fluorescence intensity of less than about 4×10³ cell surface expressed CD150 proteins per cell wherein CD150^{low} HSCs comprise the lowest 25% subpopulation of a Long-Term-HSC population which expresses a Lin⁻Sca-1⁺c-Kit⁺CD48⁻CD34⁻CD150⁺ cell surface biomarker signature based on flow cytometry or fluorescence activated cell sorting (FACS) analysis and/or express marker genes Rnase6 and Arhgap30, in the manufacture of a medicament for treating an aging or aged subject having hematopoietic stem cells expressing a high abundance of the CD150 glycoprotein (CD150^{high} HSCs); wherein the CD150^{high} HSCs have a mean fluorescence intensity of greater than about 4×10³ cell surface expressed CD150 proteins per cell or wherein CD150^{high} HSCs comprise the highest 25% subpopulation of a Long-Term-HSC population which expresses a Lin⁻Sca-1⁺c-Kit⁺CD48⁻CD34⁻CD150⁺ cell surface biomarker signature based on flow cytometry or fluorescence activated cell sorting (FACS) analysis; and/or wherein the CD150^{high} HSCs express a CD150 mRNA level of between about 0.3 to about 2 RNA Units per cell. In an embodiment of the use, the CD150^{low} HSCs express high or increased levels one or more genes selected from Serpinb1a, Usp6nl, Lrr1, Col4a2, Gng2, Kif15, Rnase6, or Arhgap30 relative to a control with low or no expression of these markers.

In another aspect, a method is provided in which a sample, such as a blood sample (e.g., a peripheral blood sample), a lymph sample, a bone marrow sample, or a tissue preparation is contacted with an anti-CD150 antibody conjugated to saporin (CD150-saporin) to eliminate CD150^{high} HSCs (e.g., potentially dysfunctional CD150^{high} HSCs having increased cell-surface expression of CD150) obtained from an old or aged subject, e.g., a mammalian subject, including a human subject, in the absence of transplantation. A titrated dosage of the conjugated CD150-SAP may be used to specifically deplete CD150^{high} HSCs both in vitro and in vivo. The elimination of CD150^{high} HSCs in an old or aged subject, as exemplified in mice, results in an increased B cell ratio and a decreased myeloid cell ratio in peripheral blood, which supports the targeted removal of dysfunctional or defective HSCs in old or aged subjects as an advantageous and beneficial method or procedure for mitigating aging-related functional decline.

In an aspect, a method of reducing or eliminating dysfunctional or defective CD150-expressing hematopoietic stem cells (HSCs) in a subject is provided in which the method involves contacting a sample containing CD150-expressing hematopoietic stem cells (HSCs) obtained from a subject in need thereof with an anti-CD150 antibody conjugated to a cellular toxin in an amount effective to bind to high CD150-expressing HSCs (CD150^{high} HSCs); wherein the CD150^{high} HSCs express one or more age-related marker genes selected from the group consisting of Sbspon, Ehd3, Clu, Dpy19l2, Kcnb2, Selp, B3gnt8, Jam2, Enpp5, Gpr183, and Ramp2 which correlate with high expression of CD150 on the cell surface; culturing the sample for a time sufficient for the anti-CD150 antibody-toxin conjugate to cause cytolysis of the CD150^{high} HSCs; and obtaining a sample in which dysfunctional or defective CD150-expressing hematopoietic stem cells (HSCs) are reduced or eliminated. In an embodiment, the subject is an old, aged, or aging subject. In embodiments, the anti-CD150 antibody-toxin conjugate is used in an amount selected from 0.01 to 1 nM, 0.01 to 0.05 nM, or 0.01 nM. In an embodiment, the method further involves measuring death of cells in the culture after three days or longer. In an embodiment, the method further involves measuring a decrease or reduction in the number of CD150-expressing HSCs compared to a control and/or a decreased ratio of CD150^{high} HSCs to CD150^{low} HSCs in the sample. In an embodiment, the control is a saline-, a buffer-, or a phosphate buffered saline-treated sample. In an embodiment, the sample is selected from blood, peripheral blood, cord blood, serum, plasma, or bone marrow. In an embodiment, the method further involves administering to the subject the sample in which dysfunctional or defective high CD150-expressing hematopoietic stem cells (HSCs) are reduced or eliminated. In an embodiment, the sample is isolated and/or purified by methods known and practiced in the art. In an embodiment, the method is performed in vitro or ex vivo. In an embodiment of the method, the sample in which dysfunctional or defective high CD150-expressing hematopoietic stem cells (HSCs) are reduced or eliminated is administered to the subject.

In another aspect a method of reducing or eliminating dysfunctional or defective CD150-expressing hematopoietic stem cells (HSCs) in an old, aged or aging subject is provided, in which the method involves administering to a subject in need thereof an effective amount of an anti-CD150 antibody conjugated to a cellular toxin in an amount effective to bind to high CD150-expressing HSCs (CD150^{high} HSCs) in the subject; wherein the CD150^{high} HSCs express one or more age-related marker genes selected from the group consisting of Sbspon, Ehd3, Clu, Dpy19l2, Kcnb2, Selp, B3gnt8, Jam2, Enpp5, Gpr183, and Ramp2 which correlate with high expression of CD150 on the cell surface; thereby reducing or eliminating dysfunctional or defective CD150-expressing hematopoietic stem cells (HSCs) in the subject. In an embodiment, the anti-CD150 antibody-toxin conjugate is administered in an amount of 1 mg/kg to 5 mg/kg or in an amount of 1 mg/kg. In an embodiment, the anti-CD150 antibody-toxin conjugate is administered in an amount of 1 mg/kg. In an embodiment, the method further involves measuring the percentage of high CD150-expressing HSCs (CD150^{high} HSCs) remaining after administration of the anti-CD150 antibody-toxin conjugate. In an embodiment, the method further involves measuring a decreased ratio of CD150^{high} HSCs to CD150^{low} HSCs in the subject after administration of the anti-CD150 antibody-toxin conjugate. In an embodiment, the method further involves measuring a reduction of about 60% CD150^{high} HSCs in the subject compared to a control after administration of the anti-CD150 antibody-toxin conjugate. In an embodiment of the method, the control is a saline-treated subject, a buffer-treated subject, or a phosphate buffered saline-treated subject. In an embodiment of the above-delineated method and/or embodiments thereof, reducing or eliminating the functionally defective or dysfunctional CD150^{high} HSCs attenuates an aging phenotype and improves physical functioning in the old, aged, or aging subject. In an embodiment of the above-delineated method and/or embodiments thereof, the percentage of B cells is increased and the percentage of myeloid cells are decreased in the subject by about 45-90 days following administration of the anti-CD150 antibody-toxin conjugate. In an embodiment, the measuring step is performed at least three weeks following administration of the anti-CD150 antibody-toxin conjugate.

In an embodiment of any of the above-delineated methods of reducing or eliminating dysfunctional or defective CD150-expressing hematopoietic stem cells (HSCs) and/or embodiments thereof, the CD150-expressing HSCs are characterized as Lin−Sca-1+C-Kit+CD48−CD34−CD150+. In another embodiment of the methods and/or embodiments thereof, the anti-CD150 antibody is TC15-12F12.2. In an embodiment, the anti-CD150 antibody is conjugated to saporin. In an embodiment, the saporin is conjugated to streptavidin. In an embodiment of any of the above-delineated methods of reducing or eliminating dysfunctional or defective CD150-expressing hematopoietic stem cells (HSCs) and/or embodiments thereof, the subject is 50 years old or older. In an embodiment of these methods and/or embodiments thereof, the old, aged, or aging subject has a defective or dysfunctional CD150^{high}-expressing population of HSCs associated with expression of one or more HSC age-related markers selected from Sbspon, Ehd3, Clu, Dpy19l2, Kcnb2, Selp, B3gnt8, Jam2, Enpp5, Gpr183, and Ramp2. In an embodiment of these methods and/or embodiments thereof, amounts of HSCs comprising lower CD150 levels (CD150^{low} HSCs) are increased, wherein said CD150^{low} HSCs express one or more genes selected from the group consisting of Serpinb1a, Usp6nl, Lrr1, Col4a2, Gng2, Kif15, Rnase6, or Arhgap30. In an embodiment of any of the above-delineated methods and/or embodiments thereof, administration to a subject is selected from parenteral, intravenous, or subcutaneous administration.

In embodiments of any of the above-delineated methods of reducing or eliminating dysfunctional or defective CD150-expressing hematopoietic stem cells (HSCs) and/or embodiments thereof, CD150^{low} HSCs comprise the lowest 25% subpopulation of a Long-Term-HSC population in bone marrow which expresses a Lin⁻Sca-1⁺c-Kit⁺CD48⁻CD34-CD150⁺ cell surface biomarker signature and CD150^{high} HSCs comprise the highest 25% subpopulation of a Long-Term-HSC population in bone marrow which expresses a Lin⁻Sca-1⁺c-Kit⁺CD48⁻CD34⁻CD150⁺ cell surface biomarker signature based on flow cytometry or fluorescence activated cell sorting (FACS) analysis.

Provided in another aspect is a kit or article of manufacture comprising a composition comprising an effective amount of hematopoietic stem cells (HSCs) expressing a low abundance of the CD150 glycoprotein (CD150^{low} HSCs), wherein said CD150^{low} HSCs have a mean fluorescence intensity of less than about 4×10³ cell surface expressed CD150 proteins per cell based on flow cytometry or fluorescence activated cell sorting (FACS) analysis; and/or wherein CD150^{low} HSCs express one or more genes selected from Serpinb1a, Usp6nl, Lrr1, Col4a2, Gng2, Kif15, Rnase6, or Arhgap30, which correlate with CD150^{low} expressing HSCs, and instructions for use. In an embodiment, the CD150^{low} HSCs comprise the lowest 25% subpopulation of a Long-Term-HSC population in bone marrow which expresses a Lin⁻Sca-1⁺c-Kit⁺CD48⁻CD34⁻CD150⁺ cell surface biomarker signature based on flow cytometry or fluorescence activated cell sorting (FACS) analysis; In an embodiment, the composition is a physiological or pharmaceutically acceptable composition.

In embodiments related to any of the above-delineated methods, compositions, and/or embodiments thereof, based on FACS analysis of LT-HSCs to determine CD150 surface expression level, the lowest 25% subpopulation (lower level of CD150) is defined as CD150^{low} HSCs, which show youthful signatures and exhibit good function. The highest 25% subpopulation (higher level of CD150) is defined as CD150^{high} HSCs, which show aging signatures and phenotypes when administered, e.g., via transplantation, into recipient mice. In an embodiment, the LT-HSCs, which comprise a source of CD150^{low} and CD150^{high} HSCs, express the following biomarker signature on their cell surface: Lin⁻Sca-1⁺c-Kit+CD48⁻CD34⁻CD150⁺. In an embodiment. HSCs are isolated and/or purified from bone marrow.

Compositions and articles as described in the aspects and embodiments herein were isolated or otherwise manufactured in connection with the examples provided below. Other features and advantages of the disclosure and embodiments will be apparent from the detailed description, and from the claims.

### Definitions

Unless defined otherwise, all technical and scientific terms used herein have the meaning commonly understood by a person skilled in the art related to the disclosure and embodiments described herein. The following references provide one of skill with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a cell, small molecule, polypeptide, polynucleotide, or a functional fragment thereof. In some embodiments, the cell is a hematopoietic stem cell (HSC) selected as having a reduced level of CD150 or a high level of CD150 relative to a reference. In some cases, the reference is a non-CD150-expressing cell. In an embodiment, the cell is a Long-Term HSC (LT-HSC), which resides in bone marrow.

By “ameliorate” is meant decrease, reduce, suppress, attenuate, diminish, arrest, abrogate, improve, or stabilize the development or progression of a disease, a dysfunction, or a disorder.

By “alteration” is meant a change (increase or decrease) in the expression levels or activity of a gene or polypeptide as detected by standard art known methods such as those described herein. As used herein, an alteration includes a 10% change in expression levels, a 20% change, a 25% change, a 40% change, or a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, “blood components” refer to hematopoietic cells or cell types, or cells or cell types derived, differentiated, or generated from hematopoietic stem cells (HSCs), in the blood, serum, plasma, and/or bone marrow of a mammalian subject or individual. Such cells include, without limitation, activated or activatable hematopoietic cells and cell types, e.g., white blood cells, such as B cells, T cells, eosinophils, neutrophils, and myeloid cells, as well as red blood cells. In an embodiment, CD150^{low} HSCs, which have improved activities and functional properties as described herein, generate significant numbers of activated or activatable hematopoietic cells and cell types, e.g., white blood cells, such as B cells, T cells, eosinophils, neutrophils, myeloid cells, as well as increase red blood cell numbers and hemoglobin levels in old, aged, or aging subjects who receive such HSCs, to boost, renew, or rejuvenate activity and numbers of the functional blood components in the subjects.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“CD150 polypeptide” (also called Signaling Lymphocyte Activation Molecule (SLAM)), e.g., human CD150, refers to a glycoprotein having at least about 85% amino acid sequence identity to GenBank Reference No. AAI32793.1, and having CD150 antibody binding activity. CD150 is expressed on the surface of hematopoietic cells, such as immature thymocytes, T cells, B cells, natural killer, and dendritic cells. The extracellular domain of CD150 is the receptor for measles virus. CD150 acts as a co-activator on T and B cells and/or as a marker of activated B cells. Both the mouse and human CD150 genes comprise seven exons spanning approximately 32 kb. (N. Wang et al., 2001, Immunogenetics, 53 (5): 382-394. CD150 embraces an amino acid sequence having at least 85%, at least 90%, at least 95%, at least 98%, at least 99%, or 100% identity to the human canonical SLAM (aka SLAMF1) amino acid sequence (GenBank Reference No. AAI32793.1 and UniProt Accession No. Q13291-1). The sequence of an exemplary CD150 polypeptide follows:

| (SEQ ID NO: 1) | |
|---|---|
| 1 | MDPKGLLSLT FVLFLSLAFG ASYGTGGRMM NCPKILRQLG SKVLLPLTYE RINKSMNKSI |
| 61 | HIVVTMAKSL ENSVENKIVS LDPSEAGPPR YLGDRYKFYL ENLTLGIRES RKEDEGWYLM |
| 121 | TLEKNVSVQR FCLQLRLYEQ VSTPEIKVLN KTQENGTCTL ILGCTVEKGD HVAYSWSEKA |
| 181 | GTHPLNPANS SHLLSLTLGP QHADNIYICT VSNPISNNSQ TFSPWPGCRT DPSETKPWAV |
| 241 | YAGLLGGVIM ILIMVVILQL RRRGKTNHYQ TTVEKKSLTI YAQVQKPGPL QKKLDSFPAQ |
| 301 | DPCTTIYVAA TEPVPESVQE TNSITVYASV TLPES |

By “CD150 polynucleotide” is meant a polynucleotide encoding a CD150 polypeptide. An exemplary polynucleotide sequence encoding a human CD150 Signaling Lymphocyte Activation Molecule (SLAM)) is provided below. CD150 embraces a nucleic acid sequence having at least 85%, at least 90%, at least 95%, at least 98%, at least 99%, or 100% identity to the human SLAM (aka SLAMF1) nucleic acid sequence (GenBank Reference No. BC132792) as follows:

| (SEQ ID NO: 2) | |
|---|---|
| 1 | ggccaggatc ccttccttct cctcattggc tgatggatcc caaggggctc ctctccttga |
| 61 | ccttcgtgct gtttctctcc ctggcttttg gggcaagcta cggaacaggt gggcgcatga |
| 121 | tgaactgccc aaagattctc cggcagttgg gaagcaaagt gctgctgccc ctgacatatg |
| 181 | aaaggataaa taagagcatg aacaaaagca tccacattgt cgtcacaatg gcaaaatcac |
| 241 | tggagaacag tgtcgagaac aaaatagtgt ctcttgatcc atccgaagca ggccctccac |
| 301 | gttatctagg agatcgctac aagttttatc tggagaatct caccctgggg atacgggaaa |
| 361 | gcaggaagga ggatgaggga tggtacctta tgaccctgga gaaaaatgtt tcagttcagc |
| 421 | gcttttgcct gcagttgagg ctttatgagc aggtctccac tccagaaatt aaagttttaa |
| 481 | acaagaccca ggagaacggg acctgcacct tgatactggg ctgcacagtg gagaaggggg |
| 541 | accatgtggc ttacagctgg agtgaaaagg cgggcaccca cccactgaac ccagccaaca |
| 601 | gctcccacct cctgtccctc accctcggcc cccagcatgc tgacaatatc tacatctgca |
| 661 | ccgtgagcaa ccctatcagc aacaattccc agaccttcag cccgtggccc ggatgcagga |
| 721 | cagacccctc agaaacaaaa ccatgggcag tgtatgctgg gctgttaggg ggtgtcatca |
| 781 | tgattctcat catggtggta atactacagt tgagaagaag aggtaaaacg aaccattacc |
| 841 | agacaacagt ggaaaaaaaa agccttacga tctatgccca agtccagaaa ccaggtcctc |
| 901 | ttcagaagaa acttgactcc ttcccagctc aggacccttg caccaccata tatgttgctg |
| 961 | ccacagagcc tgtcccagag tctgtccagg aaacaaattc catcacagtc tatgctagtg |
| 1021 | tgacacttcc agagagctga caccagagac caacaaaggg actttctgaa ggaaaatgga |
| 1081 | aaaaccaaaa tgaacactga acttggccac aggccccaag tttcctctgg cagacatgct |
| 1141 | gcacgtctgt acccttctca gatcaactcc ctggtgatgt ttcttccaca tacatctgtg |
| 1201 | aaatgaacaa ggaagtgagg cttcccaaga atttagcttg ctgtgcagtg gctgcaggcg |
| 1261 | cagaacagag cgttacttga taacagcgtt ccatctttgt gttgtagcag atgaaatgga |
| 1321 | cagtaatgtg agttcagact ttgggcatct tgctcttggc tgga |

The amino acid sequence of CD150 from Mus musculus (mouse), GenBank Accession No. AAI17100.1) is set forth below.

| (SEQ ID NO: 3) | |
|---|---|
| 1 | MDPKGSLSWR ILLFLSLAFE LSYGTGGGVM DCPVILQKLG QDTWLPLTNE HQINKSVNKS |
| 61 | VRILVTMATS PGSKSNKKIV SFDLSKGSYP DHLEDGYHFQ SKNLSLKILG NRRESEGWYL |
| 121 | VSVEENVSVQ QFCKQLKLYE QVSPPEIKVL NKTQENENGT CSLLLACTVK KGDHVTYSWS |
| 181 | DEAGTHLLSR ANRSHLLHIT LSNQHODSIY NCTASNPVSS ISRTFNLSSQ ACKQESSSES |
| 241 | SPWMQYTLVP LGVVIIFILV FTAIIMMKRQ GKSNHCOPPV EEKSLTIYAQ VQKSGPQEKK |
| 301 | LHDALTDQDP CTTIYVAATE PAPESVQEPN PTTVYASVTL PES |

A polynucleotide sequence encoding mouse CD150 Signaling Lymphocyte Activation Molecule (SLAM)) is provided below. CD150 embraces a nucleic acid sequence having at least 85%, at least 90%, at least 95%, at least 98%, at least 99%, or 100% identity to the mouse SLAM (aka SLAMF1) nucleic acid sequence (GenBank Reference No. BC117099.1) as follows:

| (SEQ ID NO: 4) | |
|---|---|
| 1 | ctcacagctg gggaccctgt ctgcgattgc tggctaatgg atcccaaagg atccctttcc |
| 61 | tggagaatac ttctgtttct ctccctggct tttgagttga gctacggaac aggtggaggt |
| 121 | gtgatggatt gcccagtgat tctccagaag ctgggacagg acacgtggct gcccctgacg |
| 181 | aatgaacatc agataaataa gagcgtgaac aaaagtgtcc gcatcctcgt caccatggcg |
| 241 | acgtccccag gaagcaaatc caacaagaaa attgtgtctt ttgatctctc taaagggagc |
| 301 | tatccagatc acctggagga tggctaccac tttcaatcga aaaacctgag cctgaagatc |
| 361 | ctcgggaaca ggcgggagag tgaaggatgg tacttggtga gcgtggagga gaacgtttct |
| 421 | gttcagcaat tctgcaagca gctgaagctt tatgaacagg tctcccctcc agagattaaa |
| 481 | gtgctaaaca aaacccagga gaacgagaat gggacctgca gcttgctgtt ggcctgcaca |
| 541 | gtgaagaaag gggaccatgt gacttacagc tggagtgatg aggcaggcac ccacctgctg |
| 601 | agccgagcca accgctccca cctcctgcac atcactctta gcaaccagca tcaagacagc |
| 661 | atctacaact gcaccgcaag caaccctgtc agcagtatct ctaggacctt caacctatca |
| 721 | tcgcaagcat gcaagcagga atcctcctca gaatcgagtc catggatgca atatactctt |
| 781 | gtaccactgg gggtcgttat aatcttcatc ctagttttca cggcaataat aatgatgaaa |
| 841 | agacaaggta aatcaaatca ctgccagcca ccagtggaag aaaaaagcct tactatttat |
| 901 | gcccaagtac agaaatcagg gcctcaagag aagaaacttc atgatgccct aacagatcag |
| 961 | gacccctgca caaccattta tgtggctgcc acagagcctg ccccagagtc tgtccaggaa |
| 1021 | ccaaacccca ccacagttta tgccagtgtg acactgccag agagctgacc catataccca |
| 1081 | gtgaaaggac tttttgaagg aggatagaag aaccaaaatc cacactgaac tggaccccgg |
| 1141 | gtcccaagtt ctctgtgaca gaaactgcac atctgtaacc ttctccaatc agttccctgg |
| 1201 | tgacggatct gcacaggcat gcttatgaag tagatgagaa gtgaggcttc ctgggcatgc |
| 1261 | aacctgctct gctgctgaca ca |

CD150 is a type I transmembrane protein belonging to the immunoglobulin superfamily. Its molecular weight is between 70 kDa and 95 kDa. The extracellular region of the receptor is composed of one Ig variable-like domain and one Ig constant 2-like domain. The intracellular region of the receptor contains two intracellular tyrosine-based switch motifs (ITSMs) that interact with SH2 domain-containing proteins. The Slamf1 gene that encodes CD150 is located on the human chromosome 1 and consists of eight exons and seven introns. Alternative splicing of SLAMF1 transcripts results in several isoforms of the protein, including the conventional transmembrane isoform (mCD150), secreted isoform (sCD150) cytoplasmic isoform (cCD150), and a novel transmembrane isoform (nCD150).

Hematopoietic stem cells (HSCs), sometimes called blood stem cells, refer to multipotent immature or primitive cells that can develop into all types of blood cells, including white blood cells (lymphocytes), red blood cells, myeloid lineage cells. HSCs reside in several organs of the body, including peripheral blood (PB), or “blood” herein, bone marrow (BM), and umbilical cord blood (UCB). In an embodiment, HSCs are characterized by marker gene and/or encoded polypeptide expression. In an embodiment, HSCs are characterized by expression of lineage−Sca-1⁺c-Kit⁺ (“LSK”) CD48−CD34−CD150+ markers. In an embodiment, lineage−Sca-1⁺c-Kit⁺ (“LSK”) CD48−CD34−CD150+HSCs are referred to as Long Term (LT)-HSCs.

“CD150^{low} HSCs” refer to hematopoietic stem cells (HSCs), or a population of HSCs as described herein which express low levels of CD150 glycoprotein on their surface relative to HSCs from older, aged, or aging animals that express higher levels of CD150 (CD150^{high} HSCs). (FIGS. 3B, 3C). In some embodiments, the level of CD150 protein is detected by fluorescent intensity, e.g., using FACS or flow cytometry analysis, and a reduction in CD150 is a reduction in intensity by at least about 10%, 20%, 30%, 40%, 50%, 60%, 75%, 85%, 95% or more. In some embodiments, CD150^{low} HSCs also express a lesser abundance of mRNA encoding CD150 protein compared with CD150^{high} HSCs. (FIGS. 3B, 3C). In an embodiment, the level of CD150 mRNA is quantified by RNA-seq. In another embodiment, the level of CD150 mRNA is reduced by at least about 10%, 20%, 30%, 40%, 50%, 60%, 75%, 85%, 95% or more. As described herein, higher numbers and amounts of CD150^{low} HSCs are found in young animals (e.g., young animals include those that are from about or equal to 2-4 months of age) and were found to be in a more active state compared with CD150^{high} HSCs from older, aging animals (e.g., aged or aging animals include those that are greater than about or equal to 12 months of age, such as about or equal to 13 months or 22-24 months of age), which are in a more quiescent state. (e.g., a1, a2, FIGS. 2F, and 2H). In particular, CD150^{low} HSCs may be defined as having a mean fluorescence intensity of less than about 4×10³ (<about 4×10³) cell surface expressed CD150 proteins based on FACS analysis (FIG. 3B). In an embodiment, CD150^{low} HSCs typically express the marker genes Rnase6 and Arhgap30, based on scRNAseq analysis (FIG. 10C). In an embodiment, young subjects (individuals, mammals, patients) are characterized as having a greater number or abundance of CD150^{low} HSCs in their blood (peripheral blood (PB)) compared with older, aged, or aging subjects. In some embodiments, older, aged, or aging subjects have both CD150^{low} HSCs and CD150^{high} HSCs in peripheral blood (or a blood sample); however, older or aging subjects typically have a greater abundance or amount of CD150^{high} HSCs compared with CD150^{low} HSCs in peripheral blood (or a blood sample). In an embodiment, representative genes that are highly expressed in young HSCs (CD150^{low} HSCs) and correlate with young or youthful HSCs include, for example, one or more of Serpinb1a, Usp6nl, Lrr1, Col4a2, Gng2, Kif15, Rnase6 and Arhgap30 (FIGS. 10A, 11D).

“CD150^{high} HSCs” refer to hematopoietic stem cells (HSCs), or a population of HSCs as described herein which express high levels of the CD150 glycoprotein on their surface relative to HSCs from young animals that express lower levels of CD150 (CD150^{low} HSCs). (FIG. 3B). In some embodiments, the level of CD150 protein is detected by fluorescent intensity, and an increase in CD150 is an increase in intensity of at least about 5%, 10%, 20%, 30%, 40%, 50%, 60%, 75%, 85%, 95% or more. CD150^{high} HSCs also express a greater abundance of mRNA encoding CD150 protein compared with CD150^{low} HSCs. (FIG. 3C). In an embodiment, the level of CD150 mRNA is quantified by RNA-seq. In another embodiment, the level of CD150 mRNA is increased by at least about 10%, 20%, 30%, 40%, 50%, 60%, 75%, 85%, 95% or more. As described herein, higher numbers and amounts of CD150^{high} HSCs are found in old, aging, or aged animals (e.g., of the ages as described above and in Example 9) and were found to be in a more quiescent state compared with CD150^{low} HSCs from younger animals (e.g., of the ages as described above and in Example 9), (e.g., q1-q4, FIGS. 2F, 2H, 11D, and 11F). In particular, CD150^{high} HSCs may be defined as having a mean fluorescence intensity of greater than about 4×10³ (>about 4×10³) cell surface expressed CD150 proteins based on flow cytometry or fluorescence activated cell sorting (FACS) analysis (FIG. 3B). In addition, CD150^{high} HSCs express a level of CD150 mRNA between about 0.3 to about 2 RNA Units per cell (FIGS. 2H, 11F). In an embodiment, old, aged, or aging subjects (individuals, mammals, patients) are characterized as having a greater number or abundance of CD150^{high} HSCs in their blood (peripheral blood (PB)) compared with young subjects. In some embodiments, older, aged, or aging subjects have both CD150^{low} HSCs and CD150^{high} HSCs in peripheral blood (or a blood sample); however, older or aging subjects typically have a greater abundance or amount of CD150^{high} HSCs compared with CD150^{low} HSCs in peripheral blood (or a blood sample). In an embodiment, representative genes that are highly expressed in old, aged, aging HSCs (CD150^{high} HSCs) and correlate with old or aging HSCs include, for example, one or more of Sbspon, Ehd3, Clu, Dpy19l2, Kcnb2, Selp, Mt1, B3gnt8, Jam2, Enpp5, Gpr183, and Ramp2 (FIG. 11D). In an embodiment, representative genes that are highly expressed in old, aged, aging HSCs (CD150^{high} HSCs) and correlate with old or aging HSCs include, for example, one or more of Clu, Selp, Mt1, Ramp2 and Gpr183.

Based on FACS analysis of LT-HSCs to determine CD150 surface expression level, the lowest 25% subpopulation (lower level of CD150) is defined as CD150^{low} HSCs, which show youthful signatures and exhibit good function. The highest 25% subpopulation (higher level of CD150) is defined as CD150^{high} HSCs, which show aging signatures and phenotypes when administered, e.g., via transplantation, into recipient mice. In an embodiment, the LT-HSCs, which comprise a source of CD150^{low} and CD150^{high} HSCs, express the following biomarker signature on their cell surface: Lin⁻Sca-1⁺c-Kit⁺CD48⁻CD34⁻CD150⁺. In an embodiment, HSCs are isolated and/or purified from bone marrow.

“Detect” refers to identifying the presence, absence or amount of the analyte to be detected. CD150 polypeptides and polynucleotides are exemplary analytes.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include diseases or disorders involving hematopoietic stem cells, or cells of the immune lineage, e.g., lymphocytes, T lymphocytes, B lymphocytes, myeloid cells,

By “effective amount” is meant the amount of a required to ameliorate the symptoms of a disease, disorder, state, or condition, relative to an untreated patient. The effective amount of active compound(s) used to practice methods as described herein for therapeutic treatment varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA nucleic acid, or a functional portion thereof, an oligonucleotide, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein. In an embodiment, the level of CD150 polypeptide or polynucleotide is reduced using an inhibitory polynucleotide that targets a CD150 polynucleotide, thereby reducing its expression or the expression of the encoded CD150 polypeptide.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide as described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule as described herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide as described herein that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide as described herein. An isolated polypeptide as described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein, polynucleotide, analyte, or clinical indicator having an alteration, e.g., an alteration in expression level or activity, that is associated with a disease or disorder, or a state or condition. A marker can be a protein, polypeptide, peptide, or polynucleotide that may be detected and/or quantified in a sample obtained from a subject, e.g., a subject having a disease, disorder, state, or condition that is associated with the presence of the marker. In an embodiment, the marker can be CD150 expression in hematopoietic stem cells (HSCs). In embodiments, the HSCs can express low (decreased relative to a control) or high (increased relate to a control) levels of CD150, e.g., CD150^{low} HSCs or CD150^{high} HISCs. In embodiments, the marker positively correlates with CD150 expression; such markers include, but are not limited to, Sbspon, Ehd3, Clu, Dpy19l2, Kcnb2, Selp, B3gnt8, Jam2, Enpp5, Gpr183, and Ramp2 (aging-related gene expression markers). In other embodiments, the marker negatively correlates with CD150 expression; such markers include, but are not limited to, Serpinb1a, Usp6nl, Lrr1, Col4a2, Gng2, Kif15, Rnase6, or Arhgap30 (young or youthful-related gene expression markers).

As used herein, “obtaining” as in “obtaining an agent” includes synthesizing, isolating, extracting, purifying, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition. In one embodiment, the level of CD150 polypeptide or polynucleotide in an HSC derived from an aging subject is compared to the level of CD150 polypeptide or polynucleotide in an HSC derived from a young, healthy control subject. In other embodiments, the reference is a reference value. For example, the level of CD150 polypeptide or polynucleotide in an HSC is compared to a reference value based on results obtained in a population of HSCs derived from young, healthy control subjects, where the reference value defines a range of test values (e.g., normal range).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

“Rejuvenation” in one embodiment refers to the provision (e.g., administration, engraftment) of CD150^{low} HSCs to a subject, e.g., having CD150^{high} HSCs, typically characteristic of an old, aged, or aging subject as described herein, by administering an effective amount of the CD150^{low} HSCs to the subject. The administration of CD150^{low} HSCs to the subject serves to dilute or reduce the numbers of CD150^{high} HSCs in the subject, such that the CD150^{low} HSCs, having improved activities and functional properties, generate significant numbers of activated or activatable hematopoietic cells and cell types, e.g., white blood cells, such as B cells, T cells, eosinophils, neutrophils, myeloid cells, as well as increase red blood cell numbers and hemoglobin levels in the old, aged, or aging subjects, to boost, renew, or rejuvenate activity and numbers of functional blood components in such subjects. In an embodiment, rejuvenation refers to reducing, depleting, or eliminating a population of dysfunctional or diseased HCS, i.e., CD150^{high} HSCs, in vitro or ex vivo from a sample (e.g., bone marrow or blood) obtained from a subject, or in vivo by administering an antibody that reduces, depletes, ablates, or eliminates a population of dysfunctional or diseased HCS, i.e., CD150^{high} HSCs, resulting in an increase in a population of more youthful CD150^{low} HSCs in the sample or the subject, an improvement in the aging status of the cells in the sample or of the subject (e.g., a decrease in the number of myeloid cells and a corresponding decrease in the number of lymphoid cells), and/or improved physical functions as described herein.

“Saporin” refers to ribosome-inactivating protein of type 1, which inhibits protein synthesis in animal cells. The protein is useful as an immunotoxin for pharmacological applications. In an embodiment, saporin can be conjugated to an antibody, e.g., an anti-CD150 antibody, to produce an antibody-drug conjugate. An exemplary saporin polypeptide sequence is provided below. Saporin embraces an amino acid sequence having at least 85%, at least 90%, at least 95%, at least 98%, at least 99%, or 100% identity to the ribosome-inactivating protein saporin-6 sequence having Uniprot designation P20656, as follows:

(SEQ ID NO: 5)
MKIYVVATIAWILLQFSAWTTTDAVTSITLDLVNPTAGQYSSFVDKIRN
NVKDPNLKYGGTDIAVIGPPSKEKFLRINFQSSRGTVSLGLKRDNLYVV
AYLAMDNTNVNRAYYFRSEITSAESTALFPEATTANQKALEYTEDYQSI
EKNAQITQGDQSRKELGLGIDLLSTSMEAVNKKARVVKDEARFLLIAIQ
MTAEAARFRYIQNLVIKNFPNKFNSENKVIQFEVNWKKISTAIYGDAKN
GVFNKDYDFGFGKVRQVKDLQMGLLMYLGKPKSSNEANSTVRHYGPLKP
TLLIT

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide as described herein, but which does not substantially recognize and bind other molecules in a sample, for example, a biological sample, which naturally includes a polypeptide as described. A biological sample may include blood, peripheral blood, serum, plasma, lymph, bone marrow, tissue or tissue preparation, saliva, tears, urine, stool, sputum, lavage, and the like. In an embodiment, an anti-CD150 antibody specifically binds to a CD150 molecule on an HSC, e.g., a CD150^{low} HSC, a CD150^{high} HSC, or a LT-HSC.

Nucleic acid molecules useful in the methods as described herein include any nucleic acid molecule that encodes a polypeptide as described herein or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods as described herein include any nucleic acid molecule that encodes a polypeptide as described herein or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

60%, 80% or 85%, 90%, 95% or even 99% identical at the amino acid level or nucleic acid to the sequence used for comparison. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a human patient or a non-human primate, or a bovine, equine, canine, ovine, or feline mammal. Other subject mammals may include, without limitation, rodents, rabbits, mice, goats, pigs, alpacas, or llamas.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing, abrogating, alleviating, or ameliorating a disorder and/or symptoms associated therewith. It will be appreciated that, although not precluded, treating a disorder or condition does not require that the disorder, condition or symptoms associated therewith be completely eliminated.

The terms “prevent,” “preventing,” “prevention,” “prophylactic treatment” and the like refer to reducing the probability of developing a disorder or condition in a subject who does not have, but is at risk of, or susceptible to, developing a disorder or condition, e.g., one associated with hematopoietic stem cells (HSCs), particularly, due to age.

In an aspect, methods of treating disease and/or disorders or symptoms thereof which comprise administering a therapeutically effective amount of a pharmaceutical composition comprising a cell or biological product or preparation, etc. to a subject (e.g., a mammal such as a human). Thus, one embodiment is a method of treating a subject who is aging or aged (old) to increase the number of CD150^{low} HSCs (or dilute out the number of CD150^{high} HSCs) in blood so as to provide the subject with a source of all blood cell types that differentiate from the HSCs and to improve the overall and physical health of the subject. The method includes the step of administering to the mammal a therapeutic amount of an amount of a therapeutic agent (e.g., CD150^{low} HSCs) herein sufficient to treat the disease, disorder, condition, physical state or symptom thereof, under conditions such that effective treatment occurs.

The methods herein include administering to the subject (including a subject identified as in need of such treatment) an effective amount of a composition or treatment product or the like as described herein to produce such effect. Identifying a subject in need of such treatment can be in the judgment of a subject or a health care professional and can be subjective (e.g. opinion) or objective (e.g. measurable by an assay, test, or diagnostic method to identify or select the subject as needing the treatment), genetic test, enzyme or protein marker, or Marker. The therapeutic methods (which include prophylactic treatment) in general comprise administration of a therapeutically effective amount of the cells, preparations, compositions containing the same, and the like) as described herein, to a subject (e.g., animal, human) in need thereof, including a mammal, particularly a human. Such treatment will be suitably administered to subjects, particularly humans, suffering from, having, susceptible to, or at risk for a disease, disorder, condition, or symptom thereof. Determination of those subjects “at risk” can be made by any objective or subjective determination by a diagnostic test or opinion of a subject or health care provider family history, and the like).

A method of monitoring treatment progress is encompassed herein. The method includes the step of determining a level of a diagnostic marker (Marker) (e.g., any target delineated herein or modulated by a compound herein, a protein or indicator thereof, etc.) or diagnostic measurement (e.g., screen, assay) in a subject suffering from or susceptible to a disorder or symptoms thereof associated with HSCs, particularly, CD150^{high} HISCs, in which the subject has been administered a therapeutic amount of an agent, cells, composition, preparation, etc., herein sufficient to treat the disease, condition, or symptoms thereof. The level of Marker determined in the method can be compared to known levels of Marker in either healthy normal controls or in other afflicted patients to establish the disease or condition status of the subject. In preferred embodiments, a second level of Marker in the subject is determined at a time point later than the determination of the first level, and the two levels are compared to monitor the course of the disease or condition, or the efficacy of the therapy. In certain preferred embodiments, a pre-treatment level of Marker in the subject is determined prior to beginning treatment; this pre-treatment level of Marker can then be compared to the level of Marker in the subject after the treatment commences or continues, to determine the efficacy of the treatment.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof as described herein. Any compositions or methods provided and described herein can be combined with one or more of any of the other compositions and methods provided herein.

### BRIEF DESCRIPTION OF THE DRAWINGS

FIGS. 1A-1H provide schematic diagrams, line and bar graphs, and FACS analysis plots illustrating that transplantation of young HSCs rejuvenates old mice. FIG. 1A: Diagram illustration of the competitive young (2-3 months) and old (22-24 months) HSC transplantation experiment. For the first transplantation, the ratio of young to old HSCs was 1:2 (500 young and 1,000 old) while the ratio was 1:1 (1,000 young and 1,000 old) for the second transplantation. FIG. 1B: Peripheral blood chimerism of donor HSCs at different times after the 1st and 2nd transplantation. n=6 for the first and n=3 for the second transplantation. FIG. 1C: Diagram showing the experimental design of individual transplantation of 1,000 young (3 months) or 1,000 old (22-24 months) HSCs into middle-aged recipients (13-month-old). Five months after the transplantation, a series of hematopoietic and physical tests were performed. FIG. 1D: Bar graph showing the percentage of B cells, T cells, and myeloid cells in the peripheral blood (PB) of recipient mice, n=8. FIG. 1E: Bar graph showing the absolute number of blood cells in the peripheral blood (PB) of recipient mice, n=8. FIG. 1F: FACS plot and bar plot showing the percentage of naïve T cells in CD8+ T cells from recipient mice, Naïve T cells (CD44^{low}, CD62L^{high}), Tcm (CD44^{high}, CD62L^{high}) and Tem (CD44^{high}, CD62L^{low}), n=8. FIG. 1G: Bar plot showing the epigenetic age of blood from recipient mice, n=4. FIG. 1H: Physical tests of recipient mice that received young and old HSCs. Muscle strength, motor coordination, endurance and brain function were assessed, n=8. Mean±SD, student t test, *p<0.05, **p<0.01, ****p<0.0001.

FIGS. 2A-2H provide diagrams, plots, heat maps, graphs and violin plots demonstrating that scRNA-seq reveals increased heterogeneity of old HSCs. FIG. 2A: Workflow of 10× scRNA-seq of young and old HSCs. The HSCs were sorted from young (2-3 months) and old (23 months) mice. FIG. 2B: UMAP plot showing the distribution of young and old HSCs based on scRNA-seq. Clear separation of young and old HSCs indicates transcriptional changes of HSCs during aging. As known in the art, UMAP (Uniform Manifold Approximation and Projection for Dimension Reduction) is a dimension reduction technique and algorithm known in the art for general non-linear dimension reduction. UMAP is based on manifold learning techniques generated from topological data analysis and allows for the display of many-dimensional data in two or three dimensions (2D or 3D space). Using UMAP, gene expression can be compared between cells. (Becht, E. et al., (2018), Nature Biotech., 37, 38-44). FIG. 2C: Cell cycle phase analysis of young and old HSCs based on scRNA-seq. The S-phase and G2/M-phase marker genes are from Seurat package (V4.0.2). The cell cycle phase was determined by the relative expression levels of these marker genes. If neither S-phase nor G2/M phase genes were expressed, they were classified as G0/G1 phase. FIG. 2D: UMAP plot showing unsupervised clustering of young and old HSCs. In total, 6 clusters were identified with two clusters (a1 and a2) representing active, and four clusters (q1-q4) representing quiescent cells. FIG. 2E: Heatmap showing the cluster-specific marker genes expression across the 6 clusters and their enriched GO terms. Differentially expressed genes with min.pct=0.25, logfc.threshold=0.25 among the 6 clusters were used to generate the heatmap. Well-known HSC aging related genes in clusters q1 and q2 are highlighted. Commonly identified marker genes in clusters q3 and q4 were also highlighted. FIG. 2F: Box plot showing relative expression of marker genes of q1, q2 and q3 in young and old HSCs via analysis of bulk RNA-seq. The expression level was normalized to the average expression level of the old. Plot shows the mean and 5-95 percentile. Two-sided unpaired Wilcoxon test. FIG. 2G: UMAP presentation of well-known HSC aging marker genes, Sbspon, Gpr183, Clu and Ramp2 in each of the single cells. FIG. 2H: UMAP plot and violin plot showing the calculated aging score of single cells of different clusters based on the HSC aging genes identified in bulk RNA-seq. In total, 332 up-regulated genes in old HSCs were used for aging score calculation. Two-sided unpaired Wilcoxon test. **p<0.01, ****p<0.0001.

FIGS. 3A-3I provide schematic diagrams, FACS analysis plots, heat maps, bar and line graphs, and line plot data related to the identification of CD150 as a hematopoietic stem cell (HSC) aging heterogeneity marker. FIG. 3A: Workflow for identifying heterogeneity marker genes in old HSCs. FIG. 3B: FACS plot showing expression level of CD150 in young (3 months) and old (22-24 months) HSCs. FIG. 3C: HSCs from old mice were separated into four subgroups (25% for each) based on their CD150 protein levels and subjected to bulk RNA-seq, n=3. FIG. 3D: Dot plot showing Pearson correlation between CD150 signature score and aging score based on scRNA-seq data, R=0.78. FIG. 3E: Heatmap showing changes in expression of aging-related genes with ascending level of CD150 based on bulk RNA-seq in FIG. 3C. FIG. 3F: Bar graph plot showing the epigenetic age of CD150^{low} and CD150^{high} HSCs from 22-24 months old mice, n=4, paired t test, the HSC subsets from the same mice were paired with dash line. FIG. 3G: Line plot and heatmap showing ATAC-seq signal difference between CD150^{low} and CD150^{high} HSCs in aging-related open and closed regions, respectively. FIG. 3H: Diagram of competitive transplantation to evaluate the repopulation capacity of CD150^{low} and CD150^{high} HSCs from old mice. FIG. 3I: Whole blood chimerism of CD150^{low} and CD150^{high} HSCs from 22-24 months old donor mice at different times after the 1st and 2nd transplantation. n=6 for the first transplantation, and n=3 for the second transplantation. Mean±SD, student t test, *p<0.05, **p<0.01, ****p<0.0001.

FIGS. 4A-4H provide schematic depictions, FACS analysis maps, bar graphs, and UMAP data and results showing that differentiation, but not self-renewal, is a major defect of old CD150^{high} HSCs. FIG. 4A: Diagram illustration of the transplantation experiment comparing old CD150^{low} and CD150^{high} HSCs. Donor HSCs derived HSPCs from the bone marrow were analyzed on days 7 and 14 after transplantation. FIG. 4B: Representative FACS analysis of donor HSCs derived HSPCs (left) and quantification of different cell populations 7 days after transplantation (right). LT-HSC (CD45.2 LSK (i.e., Lin−Sca1+c-Kit+), CD34⁻CD48⁻CD150⁺), ST-HSC (CD45.2 LSK, CD34⁺/CD48⁺CD150⁺) and MPPs (CD45.2 LSK, CD34⁺/CD48⁺CD150⁻) were analyzed, n=3. FIG. 4C: Diagram illustration of the competitive transplantation for evaluating the long-term differentiation of CD150^{high} HSCs. Both peripheral blood and bone marrow were analyzed five months after transplantation. FIG. 4D: Representative FACS analysis of donor HSC-derived HSPCs (left) and quantification of different cell populations (right) 5 months after transplantation, n=3. FIG. 4E: Bar graph showing the chimerism of donor HSCs in LT-HSCs, ST-HSCs, MPPs and PB 5 months after transplantation, n=3. FIG. 4F: UMAP plot showing cell distribution of HSPCs derived from CD150^{low} and CD150^{high} HSCs in scRNA-seq 14 days after transplantation. FIG. 4G: UMAP presentation of cell types predicted based on HSPC marker gene expression (left). In total, 7 different cell types were identified in HSPCs. FIG. 4H: Bar graph showing relative abundance of predicted cell types in HSPCs from mice received old CD150^{low} or CD150^{high} HSCs. Mean±SD, student t test, *p<0.05, **p<0.01, ***p<0.001.

FIGS. 5A-5G provide schematic depictions, bar graphs, FACS analysis, and survival curve data and results demonstrating that transplantation of a “youthful” subset of old HSCs alleviates aging phenotypes of old or aged mice. FIG. 5A: Diagram showing the experimental design of individual transplantation of 2,000 old CD150^{low} (25% lowest), whole-HSCs (un-selected) and CD150^{high} (25% highest) HSCs into middle-aged recipients (13-month-old), n=8. Five months after transplantation, a series of hematopoietic and physical tests were performed. FIG. 5B: Bar graph showing the percentage of B cells, T cells, and myeloid cells in the PB of recipient mice of the three groups, n=8. FIG. 5C: Bar graph showing absolute numbers of blood cells in PB of recipient mice from different transplanted groups, n=8. FIG. 5D: Representative FACS analysis of naïve T cells, central memory T cells (Tcm) and effector T cells (Tem) ratio in CD8 positive (CD8+) T cells and their quantification in recipient mice of the three groups, n=8. FIG. 5E: Physical tests of recipient mice that had received CD150^{low}, whole-HSCs and CD150^{high} HSCs. Muscle strength, motor coordination and endurance were assessed, n=8. FIG. 5F: Bar graph showing the epigenetic age of recipient mice from different groups, n=4. FIG. 5G: Survival curve showing the lifespan differences among recipient mice that had received old CD150^{low}, whole-HSCs and CD150^{high} HSCs. Each mouse received 2,000 HSCs. Log-rank (Mantel-Cox) test, n=17. For FIGS. 5B-5E, Mean±SD, one-way ANOVA, *p<0.05, **p<0.01, ***p<0.001, ****p<0.0001.

FIGS. 6A-6E provide schematic depictions, bar graphs, and FACS analysis data and results illustrating that the reduction of dysfunctional HSCs ameliorates aging phenotypes in old mice after transplantation. FIG. 6A: Diagram showing the experimental design. Individual transplantation of 500 old CD150^{low} HSCs with: 2,500 CD150^{high} HSCs (1:5 group); 1,000 CD150^{high} HSCs (1:2 group), and 0 CD150^{high} HSCs (1:0 group) into middle-aged recipients (13-months-old). Five months after transplantation, a series hematopoietic and physical tests were performed. FIG. 6B: Bar graph showing the percentage of B cells, T cells, and myeloid cells in the PB of recipient mice of the different groups. FIG. 6C: Bar graph showing absolute number of blood cells in PB of recipient mice from different transplanted groups. FIG. 6D: Representative FACS analysis of naïve T cells and effector T cells (Tem) ratio in CD8 positive T cells and their quantification in recipient mice of different groups. FIG. 6E: Bar plot showing the results of physical functional tests of recipient mice of different groups. Muscle strength, motor coordination, endurance and locomotor activity were assessed. n=6 for 1:5 and 1:2 group, n=7 for 1:0 group, Mean±SD, one-way ANOVA, *p<0.05, **p<0.01.

FIGS. 7A-7F show FACS analysis data, bar graphs, line graphs and results illustrating age-related functional decline of HSCs in mice (related to above FIGS. 1A-1H). FIG. 7A: The gating strategy for sorting LT-HSCs. Cells of the lineage Sca-1⁺c-Kit⁺ (LSK) were first gated, and LT-HSCs (CD48⁻CD34⁻CD150⁺) were gated in the LSK population. FIG. 7B: Bar graph showing the percentage of LT-HSCs in LSK HSPCs from young (2-3 months) and old (22-24 months) mice, n=10 for young, n=9 for old. FIG. 7C and FIG. 7D: Peripheral blood chimerism of donor HSCs at different times after the first (FIG. 7C) and second (FIG. 7D) transplantation, n=6 for the first and n=3 for the second transplantation. FIG. 7E: Diagram illustration of individual transplantation of young and old HSCs into young recipient mice for assessing differentiation bias. FIG. 7F: Analysis of HSC differentiation toward T cells, B cells, and myeloid cells at different times after transplantation, n=3. Mean±SD, student t test, *p<0.05, **p<0.01, ***p<0.001, ****p<0.0001.

FIGS. 8A and 8B provide FACS analysis data and bar graph results demonstrating the immune profile alterations during aging in mice (related to above FIGS. 1A-1H). FIG. 8A and FIG. 8B: Representative FACS analysis and bar graph plots showing the change in percentage of naïve, Tcm and Tem with aging in PB of mice in CD8+ T cells (FIG. 8A) and CD4+ T cells (FIG. 8B). Mean±SD, student t test, n=4, *p<0.05, **p<0.01, ***p<0.001, ****p<0.0001.

FIGS. 9A-9D provide FACS analysis data, bar graphs and pictorial guides demonstrating that transplantation of young HSCs rejuvenates old mice (related to FIGS. 1A-1H). FIG. 9A: Representative FACS analysis and bar plot showing the percentage of naïve T cells in CD4+ T cells from recipient mice, n=8. FIG. 9B: Bar plots showing the body weights of recipient mice that had received young or old HSCs, n=8. FIG. 9C: Bar plots showing the total moving distance and the ratio of duration in novel arm to familiar arm in Y maze test of recipient mice, n=8. FIG. 9D: Bar plot showing the results of the fear conditioning test. Compared to acclimation, the increased freezing time was calculated, n=8. Mean±SD, student t test, **p<0.01, ns, not significant.

FIGS. 10A-10D provide a heat map, bar graph data, UMAP data, and violin plot data and results demonstrating that scRNA-seq reveals increased heterogeneity of old HSCs (related to FIGS. 2A-2H). FIG. 10A: Heatmap showing HSC aging related genes that were identified through bulk RNA-seq analysis. Well-known HSC marker genes in public datasets are bolded. FIG. 10B: Bar graph showing the enriched GO terms in HSC aging related up and down genes identified in (FIG. 10A). FIG. 10C: UMAP showing the expression of the young HSC marker genes (Rnase6 and Arhgap30) in single cells. FIG. 10D: UMAP (left) showing the calculated aging score based on public HSC aging marker genes in each single cell. Violin plot (right) showing the aging score of cells from different clusters. Two-sided unpaired Wilcoxon test, ****p<0.0001.

FIGS. 11A-11H provide dot plots, violin plots, heat map, UMAP, and bar graph data and results showing that CD150 can serve as an aging heterogeneity marker of old HSCs (related to FIGS. 3A-3I). FIG. 11A: Dot plot showing ranking of genes based on the correlation of their expression and aging score in scRNA-seq. The top 150 highly ranked genes were highlighted. FIG. 11B: Violin plot showing expression level of potential marker genes in cluster q1, q2 and q3 by scRNA-seq. FIG. 11C: Dot plot showing the ranking of genes based on their correlation with CD150 levels in expression of the four cell groups in FIG. 3C measured by bulk RNA-seq. A total of 131 positively correlated genes (R>0.8) and 103 negatively correlated genes (R<−0.75) were identified as CD150 related genes. FIG. 11D: Heatmap showing expression changes of CD150-related genes with ascending CD150 level based on bulk RNA-seq. For positive correlated genes, previously identified HSC aging marker genes are in bold. For negative correlated genes, genes that are highly expressed in young HSCs are in bold. FIGS. 11E and 11F: Feature plot (FIG. 11E) and violin plot (FIG. 11F) showing CD150 signature scores of the six cell clusters in scRNA-seq. FIG. 11G: Representative images and bar plot showing the level of γH2AX in old CD150^{low} and CD150^{high} HSCs, n=3. FIG. 11H: Left, representative FACS plot showing the percentage of old CD150^{low} and CD150^{high} HSCs in different cell cycle phases. Right, bar graph showing the percentage of old CD150^{low} and CD150^{high} HSCs in active cell cycle (G1 and G2/S/M), n=3. For FIG. 11G and FIG. 11H, Mean±SD, student t test, *p<0.05, **p<0.01. For (F), Two-sided unpaired Wilcoxon test, ****p<0.0001.

FIGS. 12A and 12B provide graphs of transplantation results showing that old CD150^{low} HSCs are functionally superior to CD150^{high} HSCs in vivo (related to FIGS. 3A-3I). FIGS. 12A and 12B: The peripheral blood chimerism of donor HSCs at different times after transplantation in the first (FIG. 12A) and second (FIG. 12B) competitive transplantation. B cells, T cells, and myeloid cells were analyzed individually. Mean±SD, student t test, n=6 for the first and n=3 for the second transplantation, *p<0.05, **p<0.01, ****p<0.0001.

FIGS. 13A-13C provide FACS analysis plots, bar graphs, schematic diagrams, and line graphs illustrating the comparable function of CD150^{low} and CD150^{high} HSCs from young mice (related to FIGS. 3A-3I). FIG. 13A: Left, representative FACS plots showing the percentage of young CD150^{low} (25% lowest) and CD150^{high} (25% highest) HSCs in different cell cycle phases. Right, bar graph showing the average percentage of young CD150^{low} and CD150^{high} HSCs in active cell cycle (G1 and G2/S/M), n=3. FIG. 13B: Diagram illustrating the competitive transplantation for evaluating the repopulating capacity of CD150^{low} and CD150^{high} HSCs from young mice. The ratio of competitor to donor HSCs to was 1:2 (300 competitor HSCs with 600 donor HSCs). FIG. 13C: The peripheral blood chimerism of donor HSCs at different times after transplantation. Whole blood, T cells, B cells, and myeloid cells were analyzed. n=3, Mean±SD, student t test, *p<0.05, **p<0.01, ns, not significant.

FIGS. 14A-14K provide schematic diagrams, FACS analysis plots, Venn diagrams, bar graphs, UMAP data, and plots showing that differentiation, but not self-renewal, is a major defect of old CD150^{high} HSCs (related to FIGS. 4A-4H). FIG. 14A: Schematic diagram of individual transplantation of old HSCs to study the differentiation trajectory after transplantation. The donor HSCs derived from HSPCs in bone marrow were analyzed on days 4, 7, 10 and 14 after transplantation. FIG. 14B: FACS analysis results showing the differentiation trajectory of old HSCs after transplantation. On the 4th day, most donor HSCs were still maintained as LT-HSCs. From day 7 and beyond, the LT-HSCs started to differentiate toward multipotent progenitor cells (MPPs), the immediate progeny of HSCs. (See, e.g., L. Purton, 2022, Society for Hematology and Stem Cells, doi.org/10.1016/j.exphem.2022.10.005). FIG. 14C: Representative FACS analysis of donor HSCs derived HSPCs (left) and quantification of different cell populations after transplantation (right) on day 14, n=3. FIG. 14D: Diagram of transplantation to evaluate the differences in activation between old CD150^{low} and CD150^{high} HISCs on day 4 after transplantation. The sorted donor HSCs were collected for transcriptome analysis, n=3. FIG. 14E: Venn diagram showing the genes that were commonly activated in old CD150^{low} and CD150^{high} HSCs compared to freshly isolated old CD150^{low} and CD150^{high} HSCs 4 days after transplantation (left). Bar graph showing enriched GO terms of the 794 commonly activated genes (right). FIG. 14F: GSEA analysis showing that transplanted HSCs highly expressed cell cycle related genes in both CD150^{low} (left) and CD150^{high} (right) HSCs when compared with freshly isolated HSCs. FIG. 14G: Schematic diagram showing the experimental design for examining HSC proliferation in vitro. In each well, 50 GFP+ cell and 50 old CD150^{low} or CD150^{high} HSCs were co-cultured. The percentage and absolute number of old CD150^{low} and CD150^{high} HSCs were quantified 6 days after culture. FIG. 14H: Bar graph showing comparable proliferation rate of old CD150^{low} and CD150^{high} HSCs 6 days after culture. The percentage of non-GFP HSCs was shown, n=10. FIG. 14I: Bar graph showing the absolute number of old CD150^{low} and CD150^{high} HISCs 6 days after culture, n=10. FIG. 14J: UMAP and bar graph showing the distribution and percentages of cells in different cell cycle phases of HSPCs from CD150^{low} and CD150^{high} HSCs 14 days after transplantation. FIG. 14K: Violin plot showing the expression pattern of cell type marker genes, including Ly6a, Kit, Cd34, Cd48, Flt3 and Slamf1 (CD150). Mean±SD, student t test, ns, not significant.

FIGS. 15A-15B provide FACS analysis plots and bar graphs showing that transplantation of a “youthful” subset old HSCs attenuates aging phenotypes of old mice (related to FIGS. 5A-5G). FIG. 15A: Representative FACS (left) analysis of naïve T cells, Tcm and Tem ratio in CD4 positive T cells of mice from different groups and their quantification (right, bar graphs). FIG. 15B: Bar graph showing body weight of recipient mice from different groups. Mean±SD, one-way ANOVA, n=8, *p<0.05, **p<0.01, ***p<0.001, ns, not significant.

FIGS. 16A and 16B provide FACS analysis plots and bar graphs demonstrating that reducing dysfunctional HSCs ameliorates aging phenotypes in old mice post transplantation (related to FIGS. 6A-6E). FIG. 16A: Representative FACS (left) analysis of naïve T cells and Tem ratio in CD4 positive (CD4+) T cells of mice from the different groups and their quantification (right, bar graphs). FIG. 16B: Bar graph showing body weight of recipient mice from different groups. n=6 for 1:5 and 1:2 group, n=7 for 1:0 group; Mean±SD, one-way ANOVA, *p<0.05, **p<0.01, ns, not significant.

FIGS. 17A-17D provide a schematic diagram, and bar graphs demonstrating the targeted depletion of dysfunctional CD150^{high} HSCs in vitro. FIG. 17A: Diagram illustration of the CD150-SAP mediated killing principle. Bio-CD150, biotin-conjugated CD150 antibody; Strep-SAP, streptavidin-conjugated saporin. FIG. 17B: Dosage-dependent killing of cultured HSCs in vitro by CD150-SAP, measured using a cell titer assay. The TC15-12F12.2 clone of the CD150 antibody was used for CD150-SAP complex reconstitution. FIG. 17C, FIG. 17D: CD150^{high} HSC-specific elimination was achieved by careful titration of the CD150-SAP complex concentration. 300 CD150^{low} HSCs from a 17-month-old B6.SJL-Ptprcª Pepcb/BoyJ (CD45.1) mouse were mixed with 300 CD150^{high} HSCs from a 17-month-old C57BL/6J (CD45.2) mouse and the resulting cell mixture was treated with CD150-SAP, IgG-SAP or CD150 antibody for 3 days. The cell number (FIG. 17C) and percentage (FIG. 17D) of CD45.1⁺ and CD45.2⁺ cells were analyzed after the 3 day treatment. *p<0.05, **p<0.01, ***p<0.001, ****p<0.0001.

FIGS. 18A-18D provide a schematic depiction, line and bar graphs, and FACS analysis data illustrating the targeted depletion of dysfunctional CD150^{high} HSCs in vivo. FIG. 18A: Experimental scheme for assessing the persistency and labeling efficiency of HSC in mice by the bio-CD150 antibody. At 1, 3, 6, 12, 20 days, mice were sacrificed, and the CD150 signal on HSCs in the bone marrow was measured by strep-FITC (streptavidin-conjugated FITC). Bio-CD150 antibody was added to half of the sample to label the HSCs in the bone marrow. FIG. 18B: The percentage of HSCs labeled by injected bio-CD150 at different time points. FIG. 18C: The percentage of HSCs in total bone marrow cells or in the LSK (lineage−, Sca1+, cKit+) compartment 3 weeks post CD150-SAP treatment. FIG. 18D: The distribution of HSCs in relation to CD150 levels in mice with or without CD150-SAP treatment in vivo. The expression level of CD150 on the remaining HSCs was analyzed 3 weeks after different doses of CD150-SAP injection. The percentage of CD150^{low}, CD150^{med}, and CD150^{high} HSCs was calculated 3 weeks post CD150-SAP treatment. HSCs were divided into 3 groups (CD150^{low}, CD150^{med}, and CD150^{high}) with equal numbers based on the CD150 signal in PBS-injected control mice. Quantification of the three cell groups is shown by the bar-graphs. *p<0.05, **p<0.01, ****p<0.0001.

FIGS. 19A-19E provide bar graphs and FACS analysis results showing that depletion of dysfunctional CD150^{high} HSCs in vivo improves hematopoiesis. FIG. 19A: The percentage of HSCs in total bone marrow cells or in the LSK compartment 1.5 months after treatment with CD150-SAP (1 mg/kg). FIG. 19B: The expression level of CD150 on the remaining HSCs 1.5 months after treatment with CD150-SAP (1 mg/kg). The percentage of CD150^{low}, CD150^{med}, and CD150^{high} HSCs was calculated based on equal distribution of the cells in the three cell groups (CD150 low, med, and high) in PBS. FIGS. 19C-19E: The percentage of B cell (FIG. 19C), T cell (FIG. 19D), and myeloid cell (FIG. 19E) in peripheral blood analyzed at different time points after the CD150-SAP treatment. Number of mice in each group, n=6-9. In FIGS. 19C-E, the leftmost bar in the groups of bars on the x-axis for each of the delineated days represents PBS control; the second bar represents a CD150-SAP dose of 1 mg/kg; the third bar represents a CD150-SAP dose of 2 mg/kg; and the fourth bar (rightmost bar) represents a CD150-SAP dose of 4 mg/kg.

### DETAILED DESCRIPTION OF THE EMBODIMENTS

As featured and described herein, a clear heterogeneity of old (aging) hematopoietic stem cells (HSCs) exists, and this heterogeneity can be marked by the levels of CD150 glycoprotein in or expressed by the HSCs. Comparative molecular and functional analyses revealed that HSCs from old mice with low levels of CD150 (CD150^{low} HSCs) have a younger aging clock, transcriptome, and better repopulation capacity compared to HSCs having high levels of CD150 (CD150^{high} HSCs). Mechanistically, CD150^{high} HSCs from old mice have greatly compromised differentiation capacity compared to that of the CD150^{low} HSCs. In addition, decreasing the CD150^{high} HSC ratio in old mice can alleviate aging-related functional decline. Described herein are disclosure and results showing that HSC heterogeneity contributes to aging, and providing therapeutics and methods for rejuvenation and countering aging in the body.

### Characteristics of the Aging Process

The aging process is marked by a functional decline across multiple tissues and organs, including the hematopoietic system, which significantly increases the risk for a multitude of chronic diseases. Hematopoietic homeostasis declines with age, leading to deterioration in blood regeneration and the immune system response, as well as to an increase in the risk of anemia and leukemia. Interestingly, transfusion of young blood and its components into an old or aged subject's body, e.g., old or aged mice, is able to induce rejuvenation of multiple organs, including brain, muscle and liver, supporting an important role of the hematopoietic system in whole body aging. As the source of all cell types of the hematopoietic system, hematopoietic stem cells (HSCs) undergo profound functional changes with age, including increased prevalence of clonal hematopoiesis, a shift toward myeloid-biased differentiation, and a diminished capacity for blood regeneration. Recent studies have showed that replacing HSCs of old mice with those of young ones can improve health and extend the lifespan of old recipient mice (Guest, I. et al., (2015), Aging (Albany NY) 7(12): 1212-1223; Das, M. M. et al., (2019), Commun Biol, 2:73; Li, J. et al., (2019), Aging Cell, 18(6): e13026; Guderyon, M. J. et al., (2020), Aging Cell, 19(3): e13110), suggesting that HSCs play a central role in systematic aging. Transplanting aged HSCs into young mice can accelerate immune system aging and reduce their lifespan. Conversely, transplanting HSCs from young mice into old or aged recipient mice can improve the health and expand the lifespan of the old or aged recipient mice. The hematopoietic system, and particularly HSCs, are thus highlighted as key players in the aging process, suggesting that HSCs could serve as promising targets for aging-related intervention.

The function of HSCs is influenced by the bone marrow niche, and the bone marrow niche of old HSCs has been reported to restrict the functions of HSCs, as old HSCs transplanted into young recipients were found to show a better engraftment than those transplanted into old ones (Ergen, A. V. et al. 2012, Blood 119(11): 2500-2509). However, recent research studies have indicated that intrinsic alterations of HSCs with aging are associated with their functional decline, such as increased DNA damage, mitochondrial degeneration, diminished asymmetric distribution of CDC42, altered H4K16 acetylation during mitosis, and epigenetic modifications. The HSC population exhibits intrinsic heterogeneity in both young and old mice, distinguishable by lymphoid-biased, balanced, and myeloid-biased subtypes, based on surface markers and lineage predispositions. Notably, a fraction of HSCs can preserve long-term dormancy and functionality during aging. The changes of HSC heterogeneity during aging and the contribution of HSCs to systemic aging are comprehensively characterized as described herein.

As described and demonstrated herein, the transplantation of young HSCs can rejuvenate old mice. Single cell RNA sequencing (scRNA-seq) has revealed an increased aging heterogeneity in old HSCs. Systematic molecular and functional characterization indicated the presence of “youthful” and functionally defective “old” subsets of HSCs in old mice, marked by differential expression levels of CD150. Mechanistically, old CD150^{high} HSCs exhibit differentiation defects when compared to CD150^{low} HSCs. Notably, transplantation of the “youthful” subset of HSCs, e.g., CD150^{low} HSCs, into old recipient mice can attenuate aging phenotypes, including decreased epigenetic age and extended lifespan of old recipient mice when compared with old recipient mice that received old un-selected HSCs or CD150^{high} HSCs. Based on FACS analysis of LT-HSCs to determine CD150 surface expression level, the lowest 25% subpopulation (lower level of CD150) is defined as CD150^{low} HSCs, which shows youthful signatures and exhibits good function. The highest 25% subpopulation (higher level of CD150) is defined as CD150^{high} HSCs, which show aging signatures and phenotypes when administered, e.g., via transplantation, into recipient mice. In an embodiment, the LT-HSCs, which comprise a source of CD150^{low} and CD150^{high} HSCs, express the following biomarker signature on their cell surface: Lin⁻Sca-1⁺c-Kit⁺CD48⁻CD34⁻CD150⁺. In an embodiment, the LT-HSCs are derived from bone marrow. In an embodiment HSCs are isolated and/or purified from bone marrow. Importantly, reducing the dysfunctional CD150^{high} HSCs was found to attenuate aging phenotypes in old recipient mice, thus, highlighting that the removal of defective CD150^{high} HSCs from old mice could be a potential strategy for rejuvenation.

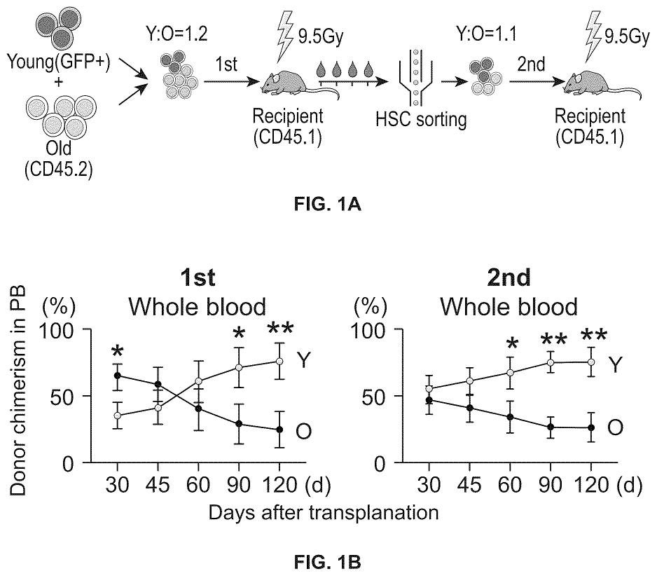

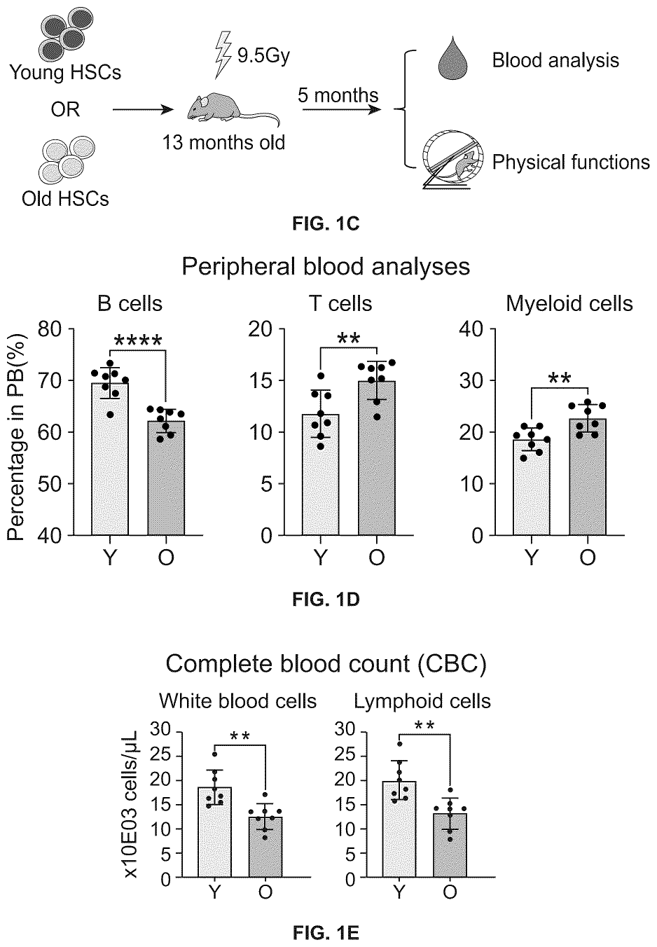

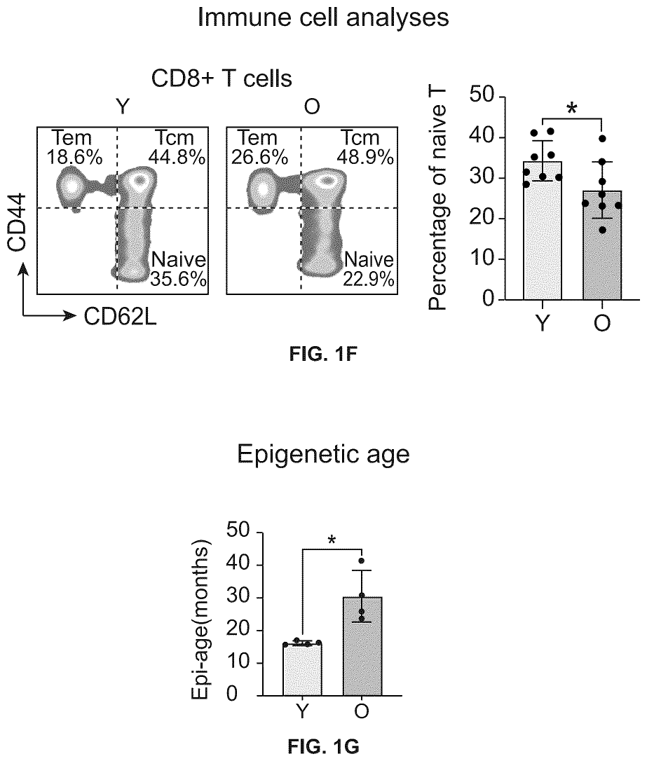

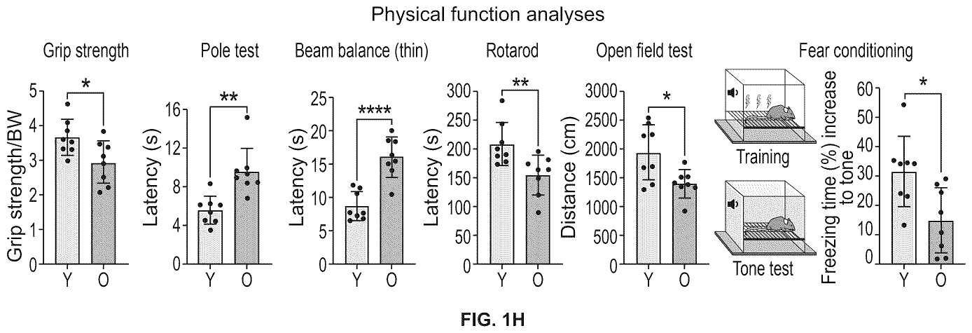

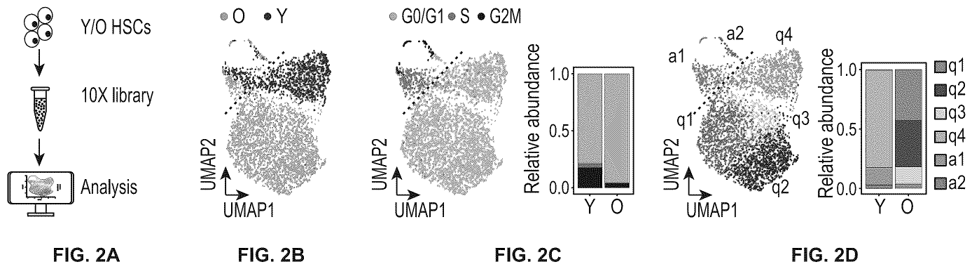

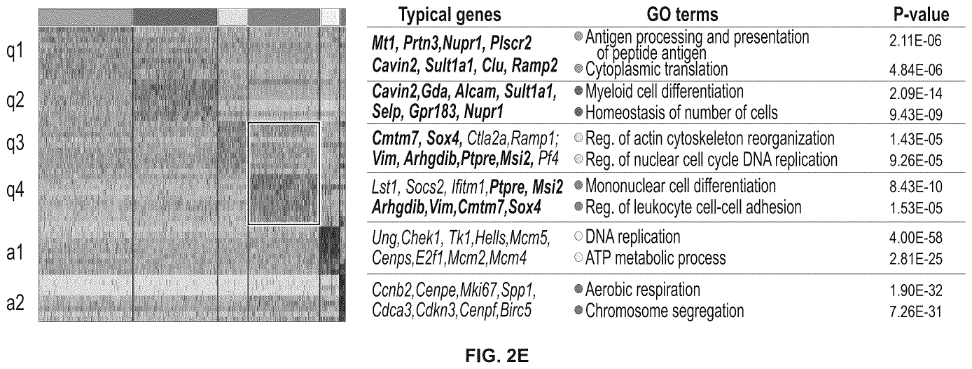

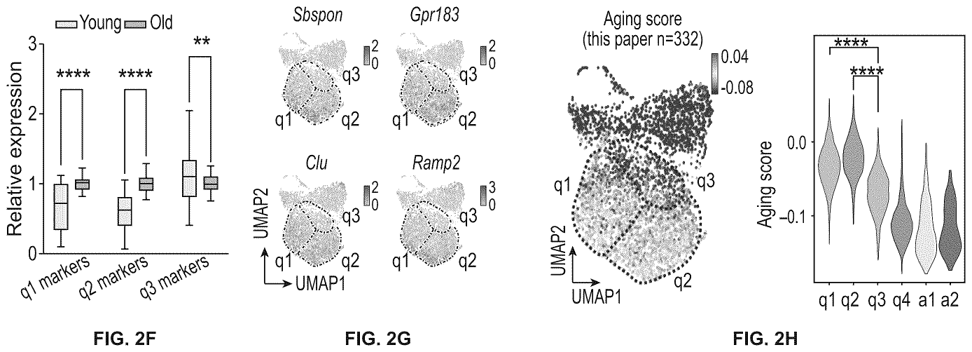

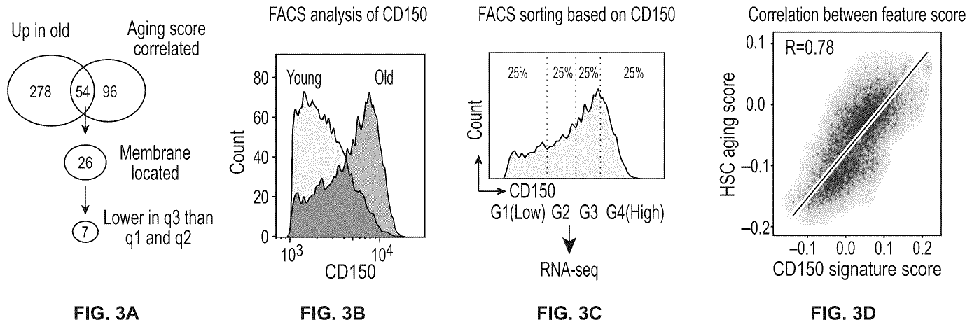

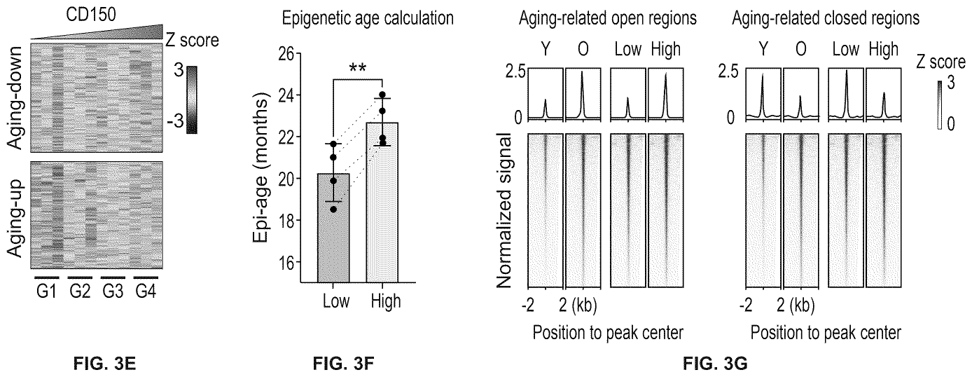

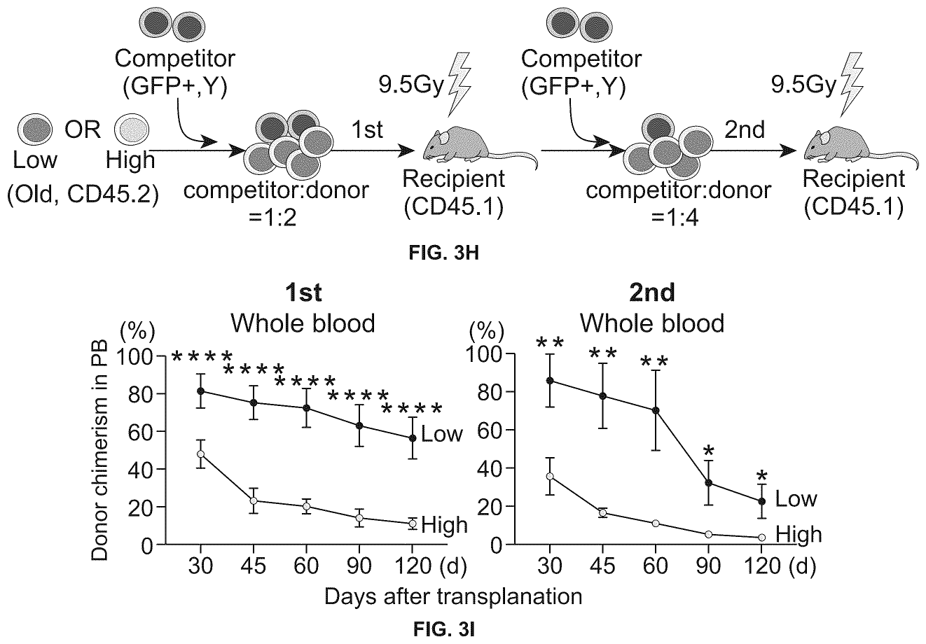

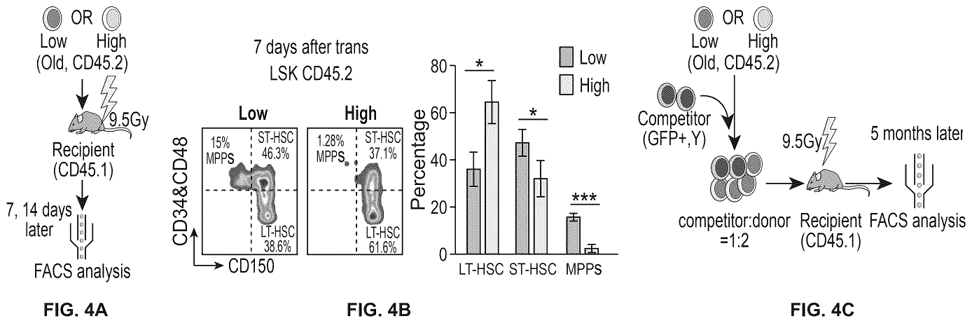

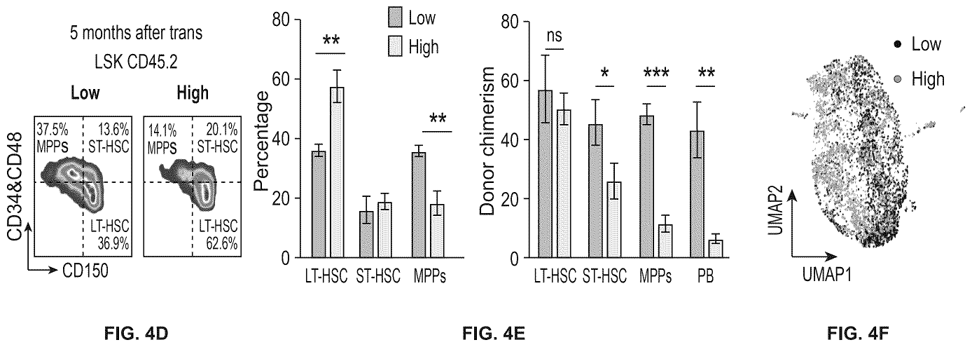

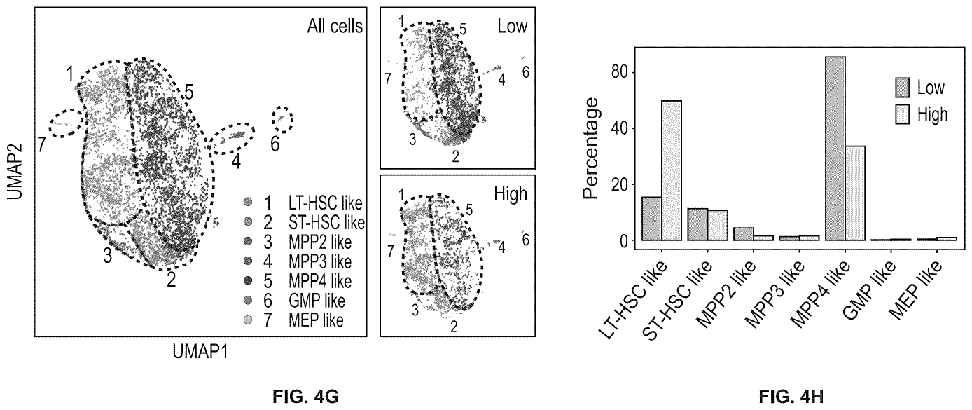

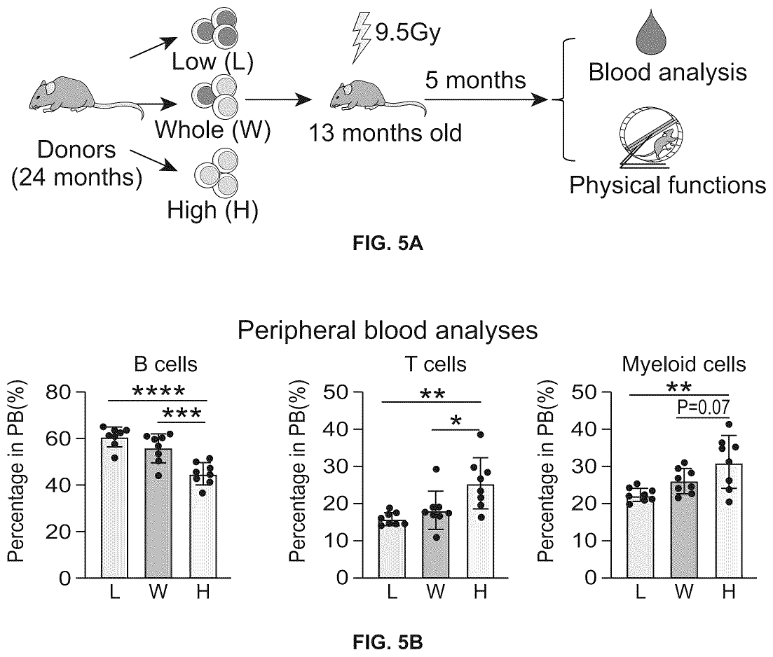

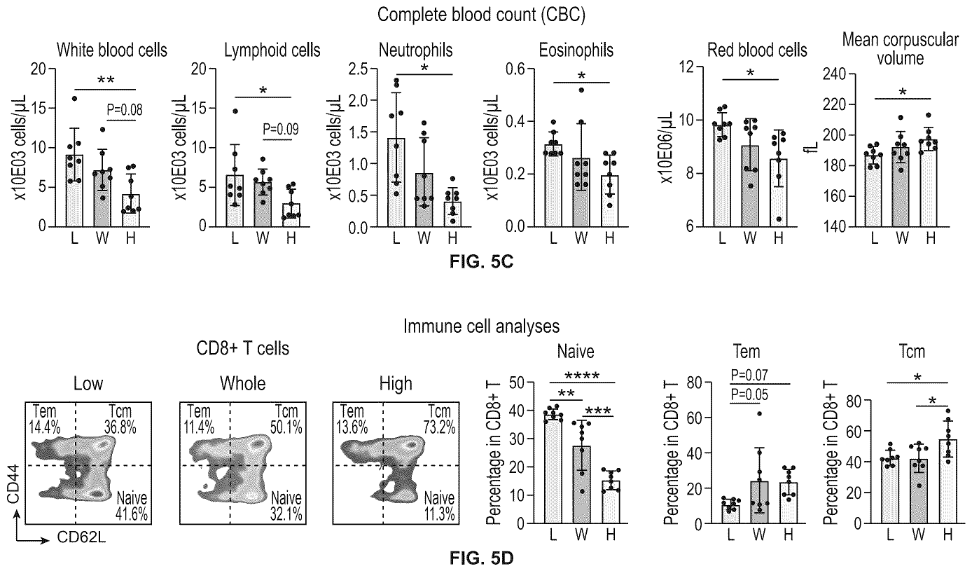

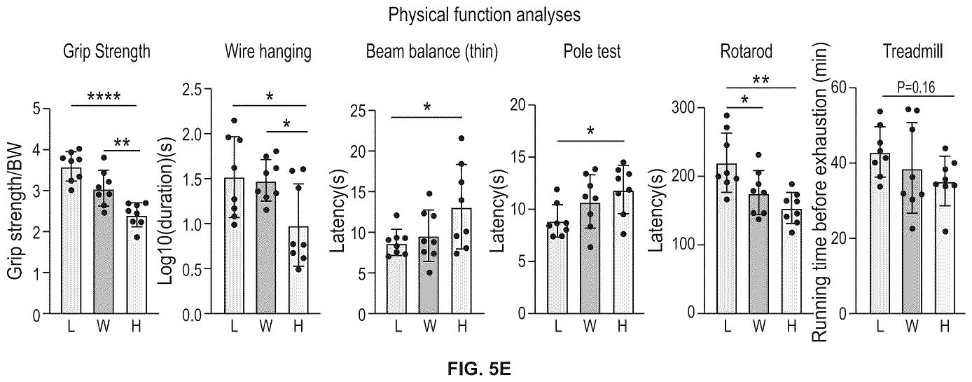

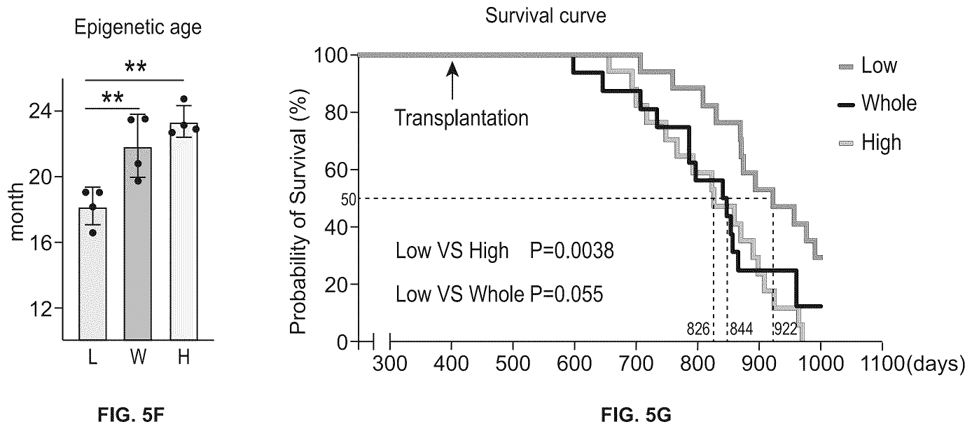

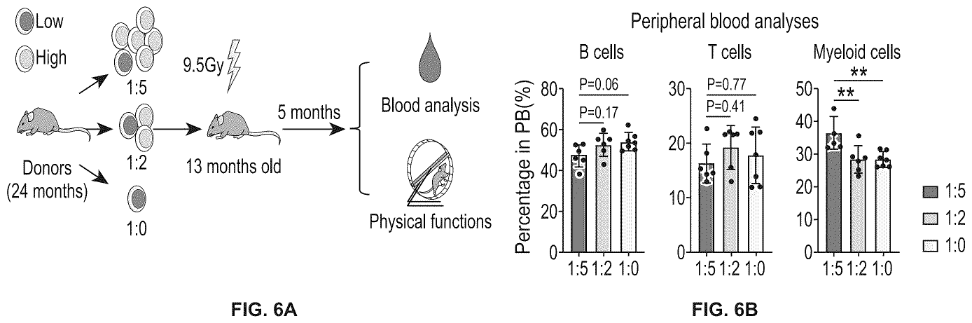

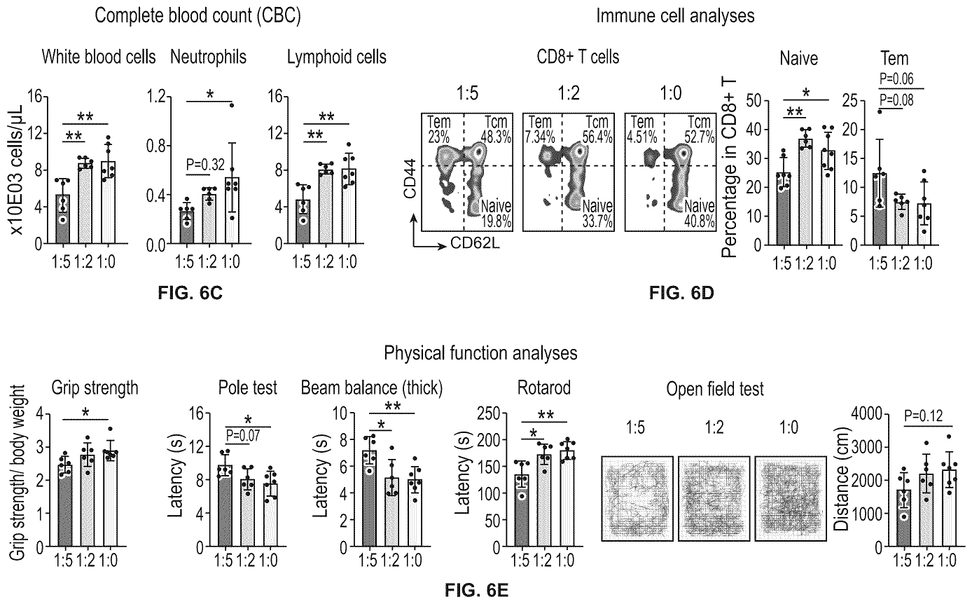

A key role of HSCs in systematic aging was demonstrated by transplantation, as described and exemplified herein (FIGS. 1A-1H). By integrating scRNA-seq and functional evaluation, a “youthful” subset of HSCs was identified in old/aged mice that is marked by a low level of CD150 (FIGS. 2A-2H and FIGS. 3A-3I). Comparative analyses demonstrated that CD150^{high} HSCs from old mice are defective in Long Term-Hematopoietic Stem Cell (LT-HSC) to Short Term-Hematopoietic Stem Cell (ST-HSC) differentiation when compared with the CD150^{low} HSCs (FIGS. 4A-4H). Importantly, transplantation of the “youthful” CD150^{low} HSCs was able to alleviate aging phenotypes and extend lifespan in old mice (FIGS. 5A-5G). As further described and demonstrated herein, the hematopoietic system and physical performance of old recipient mice was improved by reducing the ratio of functionally defective CD150^{high} HISCs in these mice (FIGS. 6A-6E), supporting the feature that rejuvenation can be achieved by removing the dysfunctional HSCs in the elderly.

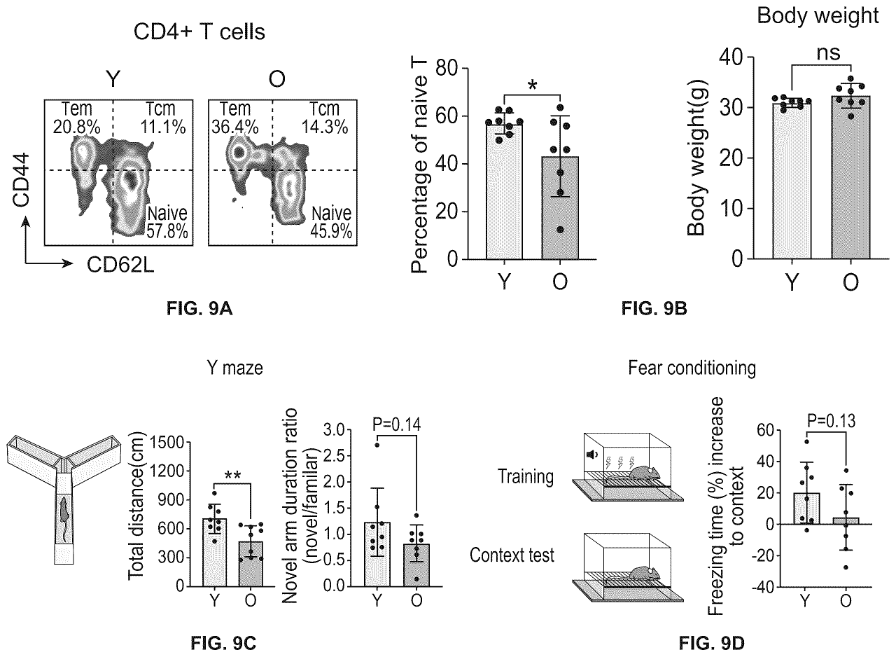

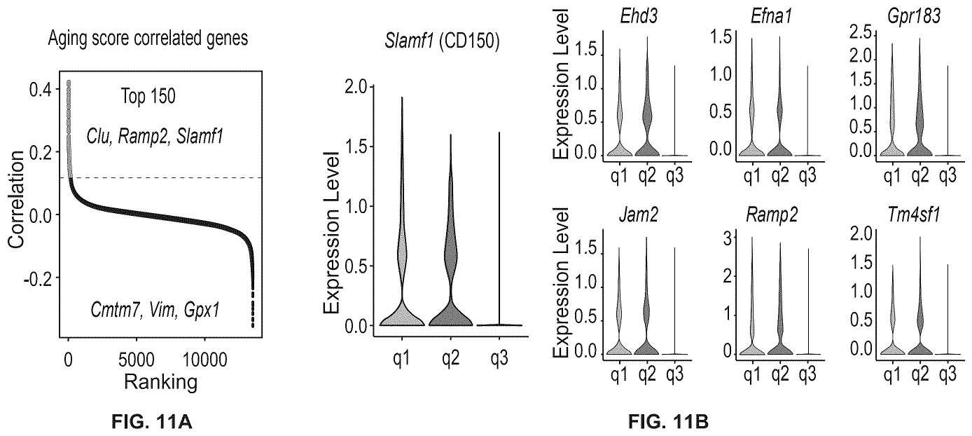

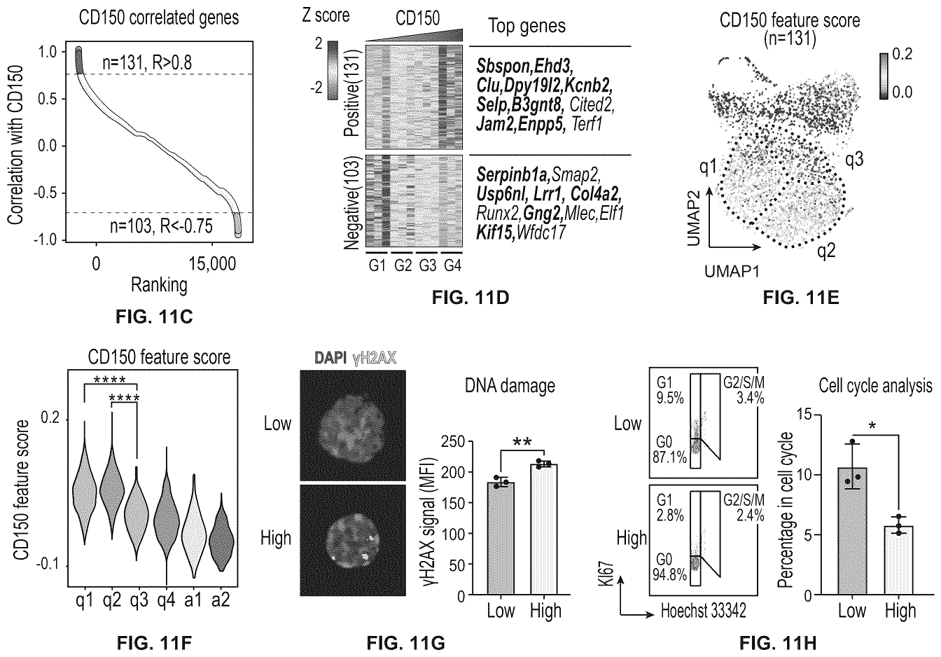

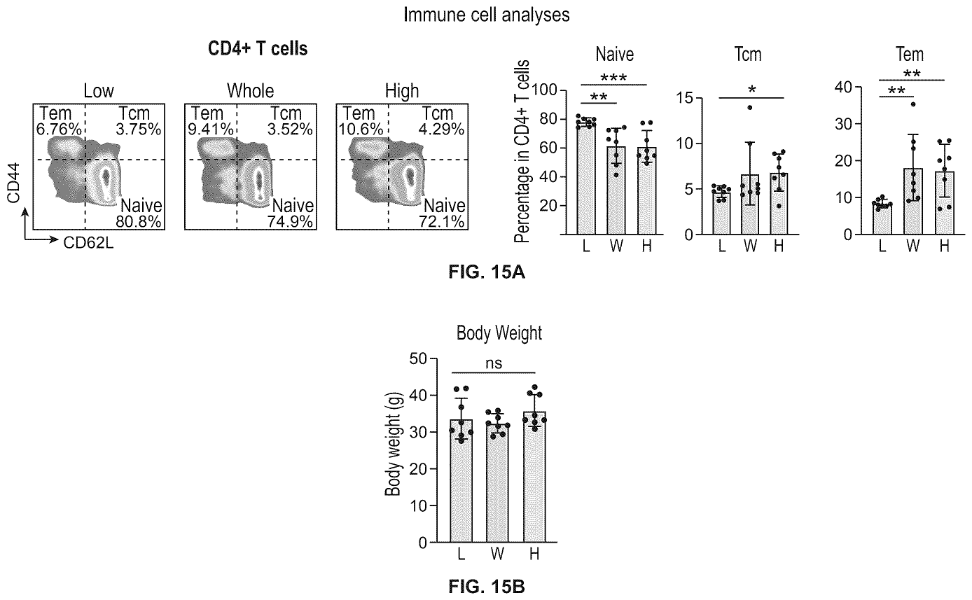

In an embodiment, young hematopoietic stem cells (HSCs) can rejuvenate an old or aging subject when transplanted into the old or aging subject as described and exemplified herein (FIGS. 1C-1H and FIGS. 9A-9D). In an embodiment, CD150 is identified as a marker of HSC aging heterogeneity as described and exemplified herein (FIG. 3G and FIGS. 11G and 11H). In an embodiment, transplantation of the “youthful” subset of old HSCs can rejuvenate an old recipient subject as described and exemplified herein (FIG. 5G). In an embodiment, the removal, elimination, deletion, depletion, or reduction of dysfunctional HSCs rejuvenates old recipient subjects in transplantation as described and exemplified herein (FIGS. 6A-6D and FIGS. 15A and 15B).

### CD150 Level as an Indicator of Aging Heterogeneity in Old HSCs

Single cell RNA sequencing (scRNA-seq) revealed that while quiescent HSCs in young mice are relatively homogeneous, the quiescent HSC counterpart in old mice are heterogenous (FIG. 2D and FIG. 2H). By analyzing the genes highly expressed in “older” subpopulations of aged HSCs, the CD150 glycoprotein (encoded by the Slamf1 gene) was identified as a cell surface marker expressed on HSCs that can serve as an indicator of HSC aging in transcriptome, epigenome and function in old HSCs (FIGS. 2A-2H and FIGS. 3A-3I). Originally characterized as a surface marker for LT-HSCs (Kiel, M. J. et al., (2005), Cell 121, 1109-1121. 10.1016/j.cell.2005.05.026), CD150 has also been used to reflect differentiation bias of HSCs in young and old mice. In addition, CD150^{low} HSCs from aged mice have been shown to retain normal lymphoid differentiation capacity when removed from the aged microenvironment (Montecino-Rodriguez, E., et al., (2019). Stem Cell Reports, 12, 584-596. 10.1016/j.stemcr.2019.01.016). These previous reports suggest a potential link between CD150 levels and HSC function during aging and are in line with the aspects and embodiments described herein, which further support CD150 as a marker of HSC aging heterogeneity.

In addition to CD150, other cell surface proteins, such as Lpar6, Ehd3 and Tm4sf1, were identified that can also mark the “older” populations in old HSCs. Clca3a1 can also serve as a marker to capture the HSC heterogeneity in old mice. In particular, Clca3a1^{high} HSCs from aged mice have a decreased repopulation capacity in primary transplantation and exhibit a myeloid-biased differentiation compared to Clca3a1^{low} HSCs (Kim, K. M. et al., (2022), Nat Commun, 13, 5187. 10.1038/s41467-022-32970-1). However, the functional difference between Clca3a1^{high} and Clca3a1^{low} old HSCs was not maintained in a second transplantation. In contrast, the functional difference between CD150^{high} and CD150^{low} old HSCs persisted in a second transplantation (FIG. 3I), indicating that CD150 is a more robust indicator of the functional heterogeneity of aged HSCs. Clca3a1 was not in the potential marker gene list as described herein due to its minimal expression difference between q3 and q1+q2 clusters in the scRNA-seq analysis.

### A “Youthful” Subset of Old HSCs is Functional in Alleviating Aging Phenotypes

As described and demonstrated herein, transplantation of young HSCs is promising for anti-aging treatment (FIGS. 1A-1H). However, due to immune rejection, transplantation of young HSCs as an anti-aging therapy may be problematic. By combining scRNA-seq with functional analysis, a “youthful” subset of HSCs that is marked by a lower level of CD150 was identified in old mice (FIGS. 2A-2H and FIGS. 3A-21). Based on FACS analysis of LT-HSCs, based on the CD150 level, the lowest 25% subpopulation (lower level of CD150) is defined as CD150^{low} HSCs, which shows youthful signatures and exhibit good function. The highest 25% subpopulation (higher level of CD150) is defined as CD150^{high} HSCs, which shows aging signatures and phenotypes when administered, e.g., via transplantation, into recipient mice. Comparative analyses further demonstrated that this subset of HSCs is superior to the whole-HSCs or CD150^{high} HSCs in terms of: 1) balanced differentiation (FIG. 5B); 2) absolute blood cells numbers (FIG. 5C); 3) immune system quality (FIG. 5D); 4) physical functions (FIG. 5E); 5) epigenetic age (FIG. 5F) and 6) lifespan (FIG. 5G). These results support the feature that transplantation of a “youthful” subset from old donors has anti-aging effects. Given that these “youthful” HSCs reside in aged individuals, immune rejection is not an issue. The demonstrated rejuvenation effect of this “youthful” HSC population found in old mice could serve as a potential source for anti-aging therapies in other mammals, including humans.

### Removal of Dysfunctional HSCs in Old Subjects as a Means of Rejuvenation

Since the discovery of the rejuvenating effects of young blood through parabiosis, great efforts have been expended searching for rejuvenation factors from young blood. Although several modulating candidate factors, such as CDC42, SELP, CCL5, PHF6, IGF1, PF4 and the Yamanaka factors (Oct4, Sox2, Klf4, c-Myc) have been reported to have rejuvenating effects during the past years, their rejuvenating capacities are limited, and the mechanisms underlying their rejuvenating effects are not clear. In some cases, such as GDF11, conflicting results have been reported. Thus far, no universally accepted rejuvenating factors have been identified. The findings described herein reveal that old HSCs exhibit molecular and functional heterogeneity, comprising “youthful” and dysfunctional “old” subsets (FIGS. 4A-4H and FIGS. 5A-5G). Moreover, reducing the proportion of dysfunctional HSCs ameliorates aging phenotypes in aged mice after transplantation (FIGS. 6A-6E), suggesting that removal of functionally defective HSCs from old subjects offers a promising approach for rejuvenation. Consistent with this notion, depletion of the myeloid-biased HSCs (marked by NEO1) by a specific antibody cocktail was reported to restore immune functions in old mice (Ross, J. B. et al., (2024), Nature, 628, 162-170. 10.1038/s41586-024-07238-x), although it is not known if this approach leads to a systemic body rejuvenation. The aspects and embodiments as described herein offer approaches to achieving rejuvenation in the elderly by targeted removal of dysfunctional HSCs.

### Targeted Depletion of Dysfunctional CD150^{high} HSCs as a Strategy to Achieve Rejuvenation in Old or Aged Mammalian Subjects

The development of translatable rejuvenation approaches for the hematopoietic system holds great promise for mitigating age-associated blood disorders and improving immune system of aged individuals. Although various interventions have been applied to rejuvenate old or aged HSCs to restore their youthful state, many of these methods are non-specific and have been met with limited success. A significant proportion of rejuvenation methods rely on transplantation, which involve irradiation and other manipulation that do not reflect real physiological conditions. The use of an anti-CD150 antibody conjugated to saporin (CD150-SAP) provides a new strategy for HSC rejuvenation by specifically targeting and removing dysfunctional, CD150-expressing HSCs in old or aged subjects via specific binding to the CD150 cell surface antigen. As described and exemplified herein, hematopoietic rejuvenation after CD150-SAP treatment was achieved in vivo in mammalian subjects.

### Methods of Use

Provided herein are methods that are useful for reducing, abrogating, checking, or slowing down the aging process. Methods are further provided for rejuvenating blood components of an aged or aging subject, or alleviating old or aging-related functional decline in an old, aging, or aged subject. In accordance with the aspects and embodiments described herein, removal or dilution of the defective HSCs, e.g., CD150^{high} HSCs, in an aged subject's body can improve the blood quality of the subject. In an embodiment, the methods involve administering to an aging or old subject an effective amount of a composition comprising CD150^{low} HSCs and a physiologically acceptable carrier, excipient or diluent, in particular, to increase the number of CD150^{low} HSCs in the aging or old subject, thereby improving the composition of the old or aging subject's blood components (blood cell types/lineages) which differentiate from the CD150^{low} HSCs.

The methods described herein may be advantageous and beneficial to rejuvenate or to improve the immune system and physical performance, particularly in aging, aged, older or elderly mammalian subjects and individuals, including humans. Without intending to be limiting, an old, older, aged, or aging individual, subject, or body, including a human subject or individual, may encompass one of a variety of ages and age ranges, for example, 40, 50, 60, 70, 80, 90 years of age or older. Ages between any of the foregoing years of age are also included. In nonlimiting embodiments, an aging, aged, old, or older subject is greater than or equal to 50 years of age, or is greater than 60, 70, or 80 years of age. In an embodiment, an old or aging subject is characterized by having a significant number or amount of CD150^{high} HSCs in peripheral blood and/or bone marrow. A young individual, subject, or body, including a human individual, subject, or body, may encompass one of a young age or age range, for example, an infant (1-12 months of age), a child (1-3, 1-5, or 1-10 years of age), or a young subject prior to adulthood (e.g., 10-21 years of age). Ages between any of the foregoing are also included. In a particular embodiment, a young subject is characterized by having a significant number or amount of CD150^{low} HSCs in peripheral blood and/or bone marrow. Without wishing to be bound by theory, and for relative comparison purposes, a mouse of 3-6 months of age, e.g., young (mature) adult mouse, may correlate with a human of 20-30 years of age; a mouse of 10-15 months of age, e.g., a middle-aged mouse, may correlate with a human of 38-47 years of age; and a mouse of 18-24 months of age, e.g., an old (elderly), aged or aging mouse, may correlate with a human of 56-69 years of age. (Hagan, C., (2017), The Jackson Laboratory Blog Post, jax.org/news and insights/jax-blog/2027/november/when-are-mice-considered-old).

Identifying or detecting CD150^{high} HSCs and other associated markers (e.g., other cell surface proteins, such as, for example, Ramp2, Gpr183, Ehd3 and Tm4sf1 that can distinguish the q3 from the q1+q2 HSC populations as described herein, and can be used to distinguish the heterogeneous population of aged HSCs) in an individual or subject may be used to identify, categorize, characterize, or select an individual or subject as being old, older, aged, or aging, and in need of an increased number of CD150^{low} HSCs in accordance with the methods and results described herein. An older individual, subject, patient, body, and the like, may be identified through testing or assay as having CD150^{high} HSCs in a blood, serum, plasma, bone marrow, or other bodily sample or fluid. Such testing may involve RNAseq, and the like, or may involve an immunoassay or other binding assay in which an antibody, e.g., an anti-CD150 antibody, e.g., a detectably labeled antibody, is used to detect expression of the CD150 glycoprotein in HSCs isolated from a sample, e.g., blood, peripheral blood (PB), serum or bone marrow, and the like. Detection of CD150 expression on cells (HSCs) and analysis thereof can be performed using flow cytometry analysis, FACS, or any other suitable detection assay used in the art.

Hematopoietic stem cells (HSCs) may be isolated from bone marrow by methods known and used in the art. CD150^{high} HSCs and CD150^{low} HSCs may be identified using labeled, detectable anti-CD150 antibodies that bind to CD150 expressed on HSCs. CD150 alone, or in combination with other surface markers, may be employed to separate HSCs from other blood cells. The identified populations of cells can be isolated via flow cytometry, for example. See, e.g., Rossi, L. et al., 2011, Methods Mol Biol, 750:47-59; Celso, C. L. 2007, J Vis Exp., (2): 157.

The methods provided herein involve administering to a subject having, or who is identified or selected as having, hematopoietic stem cells (HSCs) expressing a high level of the glycoprotein CD150 (i.e., CD150^{high} HSCs), an effective amount of HSCs expressing a lower level of CD150 (i.e., CD150^{low} HSCs). In an embodiment, the CD150^{low} HSCs are isolated from a donor subject. In an embodiment, the CD150^{low} HSCs are components of peripheral blood obtained from a subject having normal or higher than normal levels of CD150^{low} HSCs. In embodiments, CD150^{high} HSCs obtained, isolated, or purified from a sample of an older or aging subject may be contacted with an agent that reduces the amount or levels of CD150 in the subject's HSCs, such that the levels of CD150 in the HSCs is reduced and CD150^{high} HSCs are replaced or replenished by CD150^{low} HSCs. By way of nonlimiting example, the agent may be an antisense nucleic acid, polynucleotide, or oligonucleotide, an siRNA, or iRNA. In an embodiment, the subject may be treated so as to remove CD150^{high} HSCs from the blood or to dilute the number of CD150^{high} HSCs in the blood of a subject, e.g., by administering a blood sample containing CD150^{low} HSCs from a donor or a composition comprising an effective amount of CD150^{low} HSCs. In embodiments, the CD150^{low} HSCs may be autologous or allogeneic. In an embodiment, the administering is via transplantation, infusion, or immunotherapy. In an embodiment, the administered CD150^{low} HSCs serve to dilute the number of CD150^{high} HSCs in the subject, thereby resulting in dilution-based rejuvenation. In embodiments, the methods slow down the aging process, or rejuvenate; or improve the immune system and physical performance of the subject to whom CD150^{low} HSCs have been administered.

### Antibodies

As described herein, antibodies that specifically bind a marker (e.g., a cell surface moiety or protein receptor such as CD150) are useful in the methods described herein, including therapeutic and diagnostic methods. In embodiments, a nanoparticle or polymer having an scFv or antibody fragment that specifically binds a cell surface marker of a microglia and contains a therapeutic and/or diagnostic agent is provided.

Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. As used herein, the term “antibody” means not only intact antibody molecules, but also fragments of antibody molecules that retain immunogen-binding ability. Such fragments are also well known in the art and are regularly employed both in vitro and in vivo. Examples of antibody fragments include, but are not limited to, Fab, Fab′, F(ab′) 2, and Fv fragments, linear antibodies, scFv antibodies, single-domain antibodies, such as camelid antibodies (Riechmann, 1999, Journal of Immunological Methods, 231:25-38), composed of either a light chain variable (VL) or a heavy chain variable (VH) domain which exhibit sufficient affinity for the target, and multispecific antibodies formed from antibody fragments.

The antibodies for use may exist in a variety of forms including, for example, polyclonal antibodies, monoclonal antibodies, Fv, Fab and F(ab′) 2, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). For example, F(ab′) 2, and Fab fragments that lack the Fc fragment of an intact antibody, clear more rapidly from the circulation, and may have less non-specific tissue binding than an intact antibody (Wahl et al., J. Nucl. Med. 24:316-325 (1983). Thus, the antibodies may comprise, without limitation, whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062, 1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and using unconventional antibodies have been described. Bispecific antibodies produced using leucine zippers are described by Kostelny et al. (J. Immunol. 148(5): 1547-1553, 1992). Diabody technology is described by Hollinger et al. (Proc. Natl. Acad. Sci. USA 90:6444-6448, 1993). Another strategy for making bispecific antibody fragments using single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In various embodiments, an antibody is monoclonal. Alternatively, the antibody is a polyclonal antibody. The preparation and use of polyclonal antibodies are also known by the skilled artisan. Hybrid antibodies are also encompassed, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′) 2” fragment, retains both of the antigen binding sites of the intact antibody. Similarly, an antibody from which the Fc region has been enzymatically cleaved, or which has been produced without the Fc region, designated an “Fab”′ fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Methods of preparing antibodies are well known to those of ordinary skill in the science of immunology. Antibodies can be made by any of the methods known in the art utilizing a soluble polypeptide, or immunogenic fragment thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding polypeptides or immunogenic fragments thereof, can be provided to the host in a delivery vehicle that is taken up by immune cells of the host. The cells will in turn express the polypeptide thereby generating an immunogenic response in the host. Alternatively, nucleic acid sequences encoding human polypeptides or immunogenic fragments thereof, can be expressed in cells in vitro, followed by isolation of the polypeptide and administration of the polypeptide to a suitable host in which antibodies are raised.

Alternatively, antibodies may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Monoclonal antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by the described and commonly used methods can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

In embodiments, an antibody, such as an anti-CD150 antibody, is conjugated to saporin (SAP) to provide an anti-CD150 antibody-saporin (CD150-SAP) conjugate by methods known and used by those in the art and as described herein. Such an CD150-SAP conjugate is suitable for use in depleting or removing from a sample hematopoietic stem cells (HSCs) that express CD150, such as HSCs that express high levels of CD150 (CD150^{high} HSCs). In an embodiment, the anti-CD150 antibody is a monoclonal antibody that binds to/reacts with CD150 (signaling lymphocyte activation molecule or SLAM), which is an ˜75 kDa type I transmembrane glycoprotein. In an embodiment, the anti-CD150 antibody is TC15-12F12.2 (Stemcell Technologies, Cambridge, MA). In an embodiment, the saporin protein is conjugated to streptavidin.

### Pharmaceutical Compositions and Methods of Delivery

Provided in another aspect are compositions, particularly, pharmaceutically acceptable compositions or formulations, comprising the described and exemplified CD150^{low} HSCs or physiological preparations containing the CD150^{high} HSCs for use as therapeutics and for the treatment of aging, for removing or reducing defective HSCs (e.g., CD150^{high} HSCs) in an old, elderly, aging, or aged subject, sample, or body, for improving physical performance, and improving hematopoietic stem cell function, as described herein, or a symptom or side effect thereof. In a particular embodiment, a pharmaceutical composition is provided that contains CD150^{low} HSCs, isolated in a physiologically acceptable solution and/or contained in a blood preparation, as described herein.

The compositions described herein comprise a pharmaceutically-acceptable diluent, carrier, or excipient. In an embodiment, the pharmaceutically acceptable compositions are in unit dosage form. Conventional pharmaceutical practice may be employed to provide suitable formulations or compositions to administer the admixed components to aging or older patients. Administration may also begin before a patient is symptomatic. The compositions as described herein containing excipients can be prepared by any technique routinely or typically used by the practitioner in pharmacy, pharmaceutics, drug delivery, pharmacokinetics, medicine, or other related discipline in which an excipient is admixed with other components or reagents, e.g., a drug, medicament, cell population, or therapeutic agent.

Any appropriate route of administration may be employed. In general, the described methods may be practiced using any mode of administration that is medically acceptable and practicable, i.e., any mode or route that produces effective levels of the active ingredients, such as CD150^{low} HSCs disclosed herein, without causing clinically unacceptable, adverse effects. By way of nonlimiting example, modes and routes of administration include parenteral, intravenous, subcutaneous, intramuscular, intraorbital, ophthalmic, intracapsular, intrathecal, intracisternal, intranasal, intratracheal, aerosol, topical, transdermal, intravaginal, rectal (suppository), oral administration, transplantation, or within/on implants, e.g., fibers such as collagen, or osmotic pumps, etc. Therapeutic compositions may be in the form of liquid solutions or suspensions or emulsions.

Methods well known in the art for making formulations and compositions are found, for example, in “Remington: The Science and Practice of Pharmacy” Ed. A. R. Gennaro, Lippincourt Williams & Wilkins, Philadelphia, Pa., 2000, and updated editions thereof. Formulations suitable for administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful delivery systems for the compositions may include ethylene-vinyl acetate copolymer particles, osmotic pumps, implantable infusion systems, and liposomes. The formulations may contain excipients, for example, lactose, or may be aqueous solutions containing, for example, polyoxyethylene-9-lauryl ether, glycocholate and deoxycholate. In embodiments, the formulations or compositions comprise the active and inactive ingredients and components, for example, as described in Examples 1-7 herein. In embodiments, the excipients may include, without limitation, pharmaceutically acceptable components such as solvents, thickening agents, or wetting agents.

The compositions and formulations can be administered to human patients in therapeutically effective amounts (e.g., amounts which reduce, improve, or eliminate aging or an age-related condition, e.g., one that is associated with CD150^{high} HSCs) to provide therapy and treatment for aging or reducing the effects of aging, e.g., physical decline, loss of integrity of bone marrow and blood components (e.g., loss of healthy hematopoietic cells of different lineages). The preferred dosage or administered amount of a composition as described herein is likely to depend on such variables as the type and extent of the disease, disorder, or condition, the overall health status and condition of the particular patient, the formulation of the excipients, and the route of administration. In the event that a response in a subject is insufficient at the initial doses, amounts, or cell numbers administered, higher doses (or effectively higher doses by a different, more localized delivery route), or a larger number of doses, may be employed to the extent that patient tolerance permits. A single dose or multiple doses per day are contemplated to achieve appropriate systemic levels of the active ingredients, e.g., CD150^{low} HSCs, and maintenance thereof over time in the body. Buffers used herein may include acidic buffers, neutral pH buffers, and buffers meant to simulate or mimic biological pH levels.

### Kits or Articles of Manufacture

Also provided are kits or articles of manufacture for use in the described methods of counteracting the effects of aging of an old, aged, or aging subject or body, or rejuvenation of blood cell components of an old, aged, or aging patient (recipient) following CD150^{low} HSC transplantation, improving physical health, and the like. In one embodiment, the kit includes a therapeutic agent, e.g., composition comprising CD150^{low} HSCs or a sample or physiological solution containing the CD150^{low} HSCs. In an embodiment, the kit contains a composition comprising an effective amount of hematopoietic stem cells (HSCs) expressing a low abundance of the CD150 glycoprotein (CD150^{low} HSCs), wherein said CD150^{low} HSCs have a mean fluorescence intensity of less than about 4×10³ cell surface expressed CD150 proteins per cell based on flow cytometry or fluorescence activated cell sorting (FACS) analysis and/or wherein CD150^{low} HSCs express one or more genes selected from Serpinb1a, Usp6nl, Lrr1, Col4a2, Gng2, Kif15, Rnase6, or Arhgap30, which correlate with CD150^{low} expressing HSCs. In an embodiment, CD150^{low} HSCs comprise the lowest 25% subpopulation of a Long-Term-HSC population in bone marrow which expresses a Lin⁻Sca-1⁺c-Kit⁺CD48⁻CD34⁻CD150⁺ cell surface biomarker signature and CD150^{high} HSCs comprise the highest 25% subpopulation of a Long-Term-HSC population in bone marrow which expresses a Lin⁻Sca-1⁺c-Kit⁺CD48⁻CD34⁻CD150⁺ cell surface biomarker signature based on flow cytometry or fluorescence activated cell sorting (FACS) analysis. In an embodiment, the composition contains a physiologically acceptable carrier, excipient or diluent. In some embodiments, the kit comprises a sterile container which contains a therapeutic or prophylactic composition; such containers can be boxes, ampoules, bottles, vials, tubes, culture plates, bags, pouches, blister-packs, or other suitable container forms known in the art. Such containers can be made of plastic, glass, laminated paper, metal foil, or other materials suitable for holding medicaments or biological therapeutics. In an embodiment, the kit contains the composition in the form of a solution for intravenous administration.

If desired, instructions for administering the therapeutic to a subject to improve physical performance of the subject by increasing the numbers, levels, or amounts of CD150^{low} HSCs or decreasing the numbers, levels, or amounts of CD150^{high} HSCs in the subject as described herein. The instructions, if provided, will generally include information about the use of the composition. In other embodiments, the instructions include at least one of the following: description of the compositions and components; methods for using the enclosed materials for the treatment of a disease; precautions; warnings; indications; clinical or research studies; and/or references. The instructions may be printed directly on the container (when present), or as a label applied to the container, or as a separate sheet, pamphlet, card, or folder supplied in or with the container.

The following examples are put forth so as to provide those of ordinary skill in the art with a complete disclosure and description of how to make and use the aspects and embodiments as described herein, and are not intended to limit the scope of the disclosure or the embodiments as described herein.

### EXAMPLES

### Example 1: Transplantation of Young HSCs Rejuvenates Old Recipient Mice

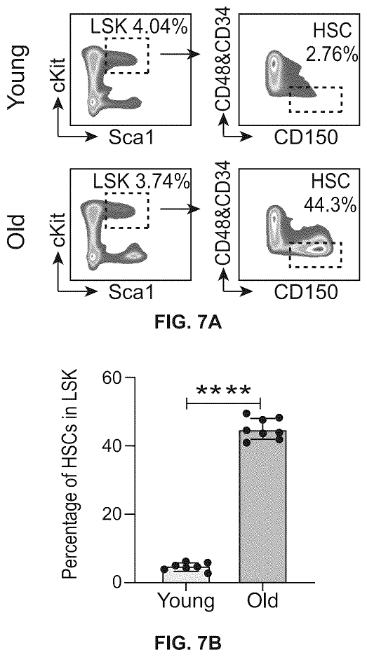

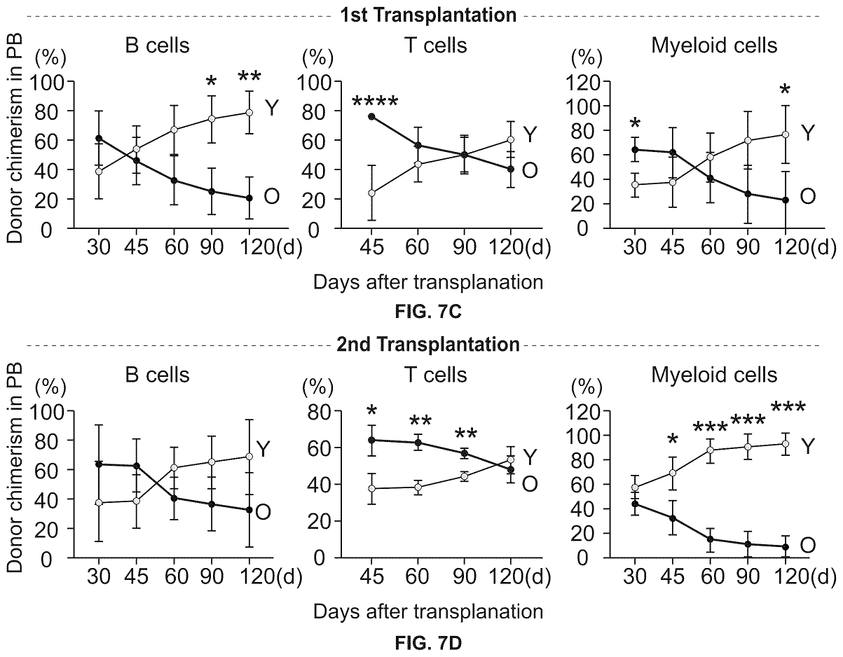

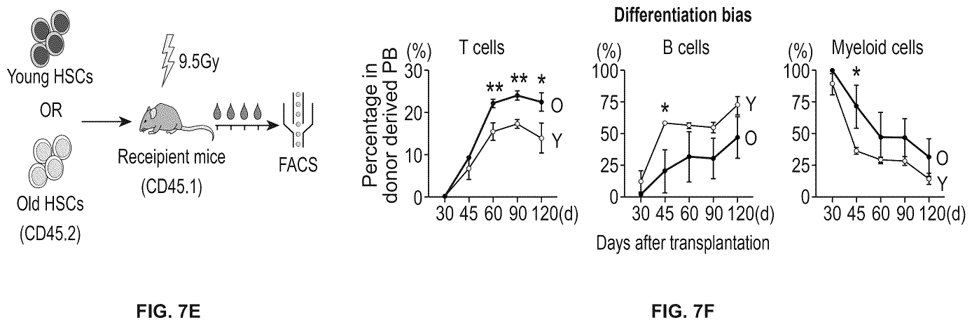

To directly evaluate the functional differences between young and old HSCs in vivo, long-term (LT)-HSCs [lineage⁻c-Kit⁺Sca-1⁺ (LSK) CD48⁻CD34⁻CD150⁺] (referred to as HSCs) were sorted from young (2-3 months old) and old (22-24 months old) mice (FIG. 7A). Consistent with previous reports (Sudo, et al. 2000, Ibid., Rossi et al. 2005. Ibid.), a significant increase in the number of HSCs in old mice was observed when compared with their young counterparts (FIG. 7B). Serial transplantation of both young and old HSCs into lethally irradiated young recipient mice (FIG. 1A), followed by examination of the donor HSC chimerism in the peripheral blood (PB), revealed a notable decline in the repopulation capacity and biased differentiation of old HSCs compared to their young counterparts (FIG. 1B and FIGS. 7C-7F). These results are consistent with previous studies (Rossi, et al. 2005, Ibid.; Leins, et al. 2018, Blood 132(6): 565-576), confirm the age-related intrinsic functional decline of HSCs.

To analyze the contribution of HSCs to systemic aging, young or old HSCs were independently transplanted into irradiated middle-aged mice (13 months). After a stable engraftment of the transplanted HSCs in the peripheral blood (5 months post-transplantation), the aging status of the recipient mice was evaluated by performing blood analyses and physical tests (FIG. 1C). Previous studies have shown that the blood cell composition changes with aging, characterized by an increase in myeloid cells and a corresponding decrease in lymphoid cells. Notably, mice transplanted with young HSCs exhibited a more youthful blood cell composition, characterized by increased B cell and reduced T cell and myeloid cell proportions, as well as an increase in overall white blood cells and lymphoid counts, compared to the recipients received old HSCs (FIG. 1D and FIG. 1E). These results suggest a crucial role of HSCs in systematic hematopoietic aging.

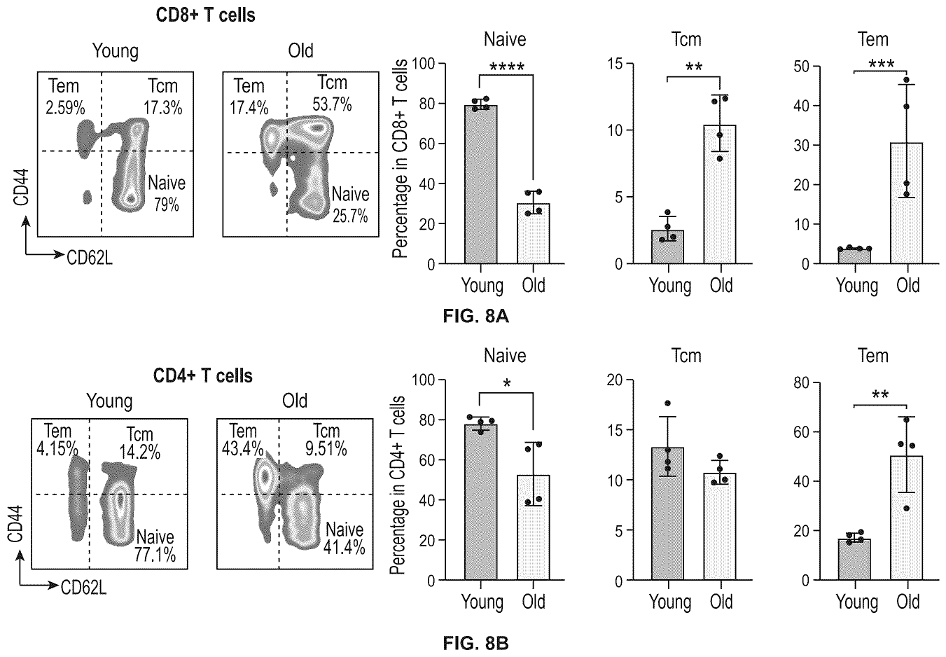

The ratio of naïve T cells serves as a critical indicator of immune function, which declines during the aging process. Conversely, central memory T cells (Tcm) and effector memory T cells (Tem) exhibit an increase with aging, as confirmed by the experiments herein (FIG. 8A and FIG. 8B). Notably, the old mice that received young HSCs had more naïve T cells in both the CD4 and CD8 T cell populations, indicating a rejuvenated immune system after young HSCs transplantation (FIG. 1F and FIG. 9A).

The DNA methylation clock is widely used as an indicator of aging. Importantly, old mice that had been transplanted with young HSCs had a significantly younger epigenetic age compared to those transplanted with old HSCs (FIG. 1G). Consistently, old mice that had received young HSCs exhibited improved physical functions when compared to those that had received aged HSCs, including muscle strength (grip strength), motor coordination (pole test, beam balance and rotarod), locomotor activity (open filed test) and cognitive functions (fear conditioning test), without significant difference in body weight of the recipients (FIG. 1H and FIGS. 9B-9D). Collectively, these results demonstrate that transplantation of young HSCs into old or aged mice can lead to whole body rejuvenation.

### Example 2: Single Cell RNA-Seq Reveals a “Youthful” HSC Subpopulation in Old Mice

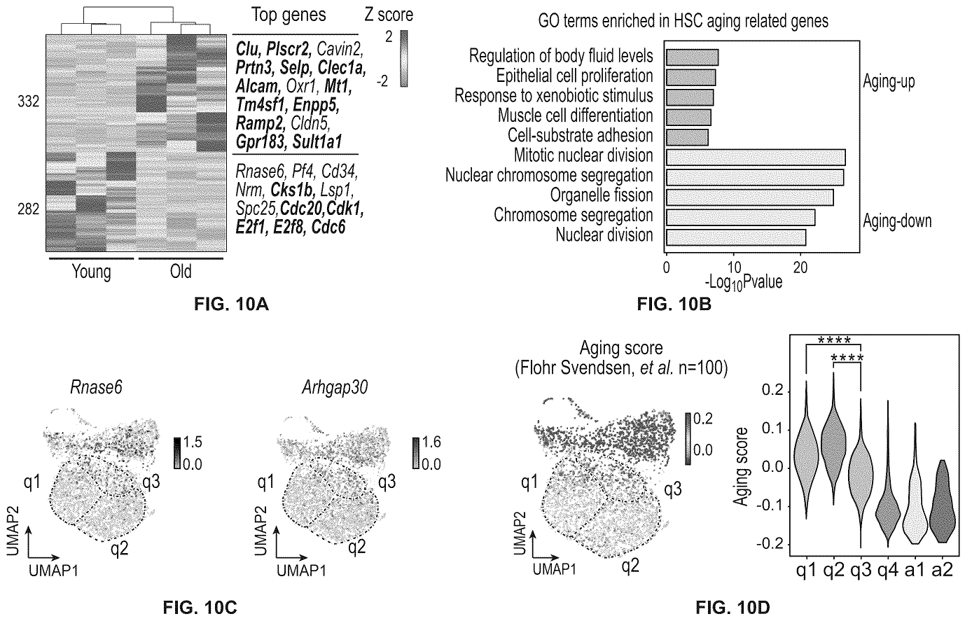

The results described in Example 1 suggested that HSCs from old mice are functionally defective compared to those from young mice. To understand the defects, both bulk RNA-seq and scRNA-seq were performed on young and old HSCs (FIG. 2A). The results revealed a clear transcriptomic difference between young and old HSCs (FIGS. 2B and 10A). Bulk RNA-seq experiments revealed 332 up-regulated genes in old HSCs, including previously annotated HSC aging marker genes such as Clu, Selp, Mt1, Ramp2 and Gpr183 (FIG. 10A; Tables 1A and 1B). These genes were defined as HSC aging marker genes for downstream analysis. Interestingly, unsupervised clustering analysis of scRNA-seq revealed that while the quiescent young HSCs are largely uniform (only form a single cluster q4), the quiescent old HSCs could be further divided into 3 clusters (q1-3) (FIG. 2C and FIG. 2D). This result indicates that quiescent old HSCs are transcriptionally more heterogeneous compared to young HSCs.

Marker genes for each of the clusters were identified (FIG. 2E). Although q1-q3 were all from old HSCs, the known aging marker genes, such as Mt1, Prtn3, Nupr1, Plscr2, Cavin2, Sult1a1, Clu, Ramp2, Alcam, Selp and Gpr183, were highly expressed in q1 and q2 clusters, but not in q3 cluster (FIG. 2E). In contrast, both q3 clusters and q4 (young HSCs) clusters shared highly expressed genes, even though the q3 cluster is from old mice. GO analysis of q3 marker genes revealed an enrichment of cell proliferation related pathways (FIG. 2E), which were also enriched in the young HSCs compared to old HSCs based on bulk RNA-seq (FIG. 10B, aging-down). The relative expression levels of q1, q2, and q3 marker genes in old and young HSCs were calculated, and it was found that the q1 and q2 marker genes have significantly higher expression in old HSCs, while expression of q3 marker genes is higher in young HSCs (FIG. 2F), indicating that q3 HSCs have a younger transcriptome than those of q1 and q2 HSCs.

To further investigate whether a subset of HSCs from old mice are “youthful” in their transcriptome, the UMAP distribution of some well-known HSC aging marker genes (Sbspon, Gpr183, Clu, Ramp2) was analyzed. It was found that these marker genes are expressed at a higher level in q1 and q2 compared to their expression in q3 (FIG. 2G). In contrast, the young HSC marker genes, such as Rnase6 and Arhgap30, exhibited an opposite expression pattern (FIG. 10C). To quantify the aging heterogeneity of old HSCs, the aging score, a measure of aging-related transcriptome, was calculated for each single cell using the 332 aging marker genes identified in the bulk RNA-seq (Tables 1A and 1B) or the top 100 HSCs aging genes as previously identified (see, e.g, Flohr Svendsen, A., et al., (2021), Blood, 138, 439-451. 10.1182/blood.2020009729.). Both analyses indicated that cluster q3 had a lower aging score than q1 and q2 despite that all cells were from the same old mice (FIG. 2H and FIG. 10D). Collectively, comparative scRNA-seq revealed increased aging heterogeneity in old HSCs and a subset of old HSCs have a youthful transcriptome.

### Example 3: CD150 can Serve as an Aging Heterogeneity Marker for Old HSCs

To better understand the aging heterogeneity of old HSCs and to assess its contribution to overall body aging, a unique cell surface marker that facilitates the separation of “youthful” from “old” cells in aged HSCs was needed. To this end, the top 150 genes showing strong correlation with aging scores in scRNA-seq were identified (FIG. 11A) and compared with the 332 genes upregulated in old HSCs (FIG. 10A), resulting in 54 shared genes (FIG. 3A; Table 2). For sorting purposes, membrane location was included, which reduced the candidate gene list to 26 (FIG. 3A). Furthermore, because the potential marker genes should have lower expression levels in q3 than in q1 and q2, the candidate gene list was reduced to 7, namely, Slamf1, Ehd, Efna1, Gpr183, Jam2, Ramp2, and Tm4sf1 (FIG. 11B). Considering the availability and specificity of the antibodies, CD150 (Slamf1) was selected as the marker for aging heterogeneity of old HSCs. FACS analysis of HSCs from young and old mice indicated that the population of HSCs with higher CD150 levels was significantly increased with aging (FIG. 3B), thus supporting the use of CD150 level to serve as an indicator of aging heterogeneity of old HSCs. By way of example, the relative fluorescent staining of CD150 is 5-fold higher in HSCs in old animals (e.g., mice) versus that in HSCs of young animals (e.g., mice). (FIG. 3B).

To further confirm that the CD150 level could serve as a marker of aging heterogeneity of old HSCs, old HSCs were separated into 4 groups (G1 to G4) by FACS, based on CD150 levels, and their transcriptomes were profiled (FIG. 3C). The expression correlation between CD150 and each gene was calculated. Based on this, 131 and 103 genes were identified that exhibited strong positive or negative correlation with CD150 level in aged HSCs, respectively (FIG. 11C; Table 3A). Notably, the genes that positively correlated with CD150 included many known HSC aging markers, such as Sbspon, Ehd3, Clu, Dpy19l2, Kcnb2, Selp, B3gnt8, Jam2 and Enpp5. In contrast, the negatively correlated genes included genes that are highly expressed in young HSCs, such as Serpinb1a, Usp6nl, Lrr1, Col4a2, Gng2 and Kif15 (FIG. 11D). These results suggested that CD150 level is a potential indicator of HSC transcriptome age.

Using the 131 genes that positively correlated with the CD150 level (referred to as CD150 feature genes), the CD150 feature score was calculated for each cell. It was found that q3 HSCs had a significantly lower CD150 feature score than q1 and q2 HSCs (FIG. 11E and FIG. 11F). Notably, a strong correlation between the CD150 feature score and aging score was observed (FIG. 3D). This indicates that the aging heterogeneity of old HSCs can be well reflected by CD150 feature genes. Consistently, HSC aging-related up- and down-regulated genes in the 4 groups of HSCs from G1 to G4 exhibited a trend that is similar to the aging process (FIG. 3E), confirming the positive correlation between CD150 level and HSC aging status in old mice. Other studies indicated that CD150 level can mark HSC subsets with different differentiation bias in both young and old mice (Morita, Y., et al., (2010), J Exp Med, 207, 1173-1182. 10.1084/jem.20091318; Beerman, I. et al., (2010), Proc Natl Acad Sci USA, 107, 5465-5470. 10.1073/pnas. 1000834107), provided support for the selection of CD150 to mark the aging heterogeneity of old HSCs.

### Example 4: CD150^{low} HSCs have a Younger Epigenome with Superior Functions in Old Mice

Epigenetic changes are believed to be one of the drivers of the aging process. To further determine whether the CD150 level can reflect epigenetic aging status in old HSCs, the epigenetic age of old CD150^{high} and CD150^{low} HSCs was compared by analyzing their DNA methylation profiles. Notably, CD150^{low} HSCs had a significantly lower epigenetic age than that of CD150^{high} HSCs, despite that they are derived from the same old mice (FIG. 3F). Given that stem cell aging is accompanied by chromatin accessibility changes, the chromatin accessibility landscapes were compared between old CD150^{low} and CD150^{high} HSCs, as well as young and old HSCs by performing ATAC-seq analyses. It was found that 4,694 peaks displayed increased chromatin accessibility and 3,842 peaks showed decreased accessibility in aged HSCs relative to young HSCs. Interestingly, a similar change between CD150^{low} and CD150^{high} old HSCs was also observed around the aging-related open and closed ATAC-seq peaks (FIG. 3G). These results indicate that CD150 can serve as a marker reflecting both transcriptional and epigenetic aging in aged HSCs.

Previous studies have shown that increased DNA damage and decreased percentage of cells in proliferation are associated with functional decline of old HSCs. Interestingly, an increase of double stranded breaks (indicated by gH2AX) as well as a decrease in the proportion of cells in proliferation for CD150^{high} was observed in the studies conducted in the described experiments compared to CD150^{low} old HSCs (FIG. 11G and FIG. 11H). This indicates that CD150^{low} HSCs may have better function than CD150^{high} HSCs in old mice.

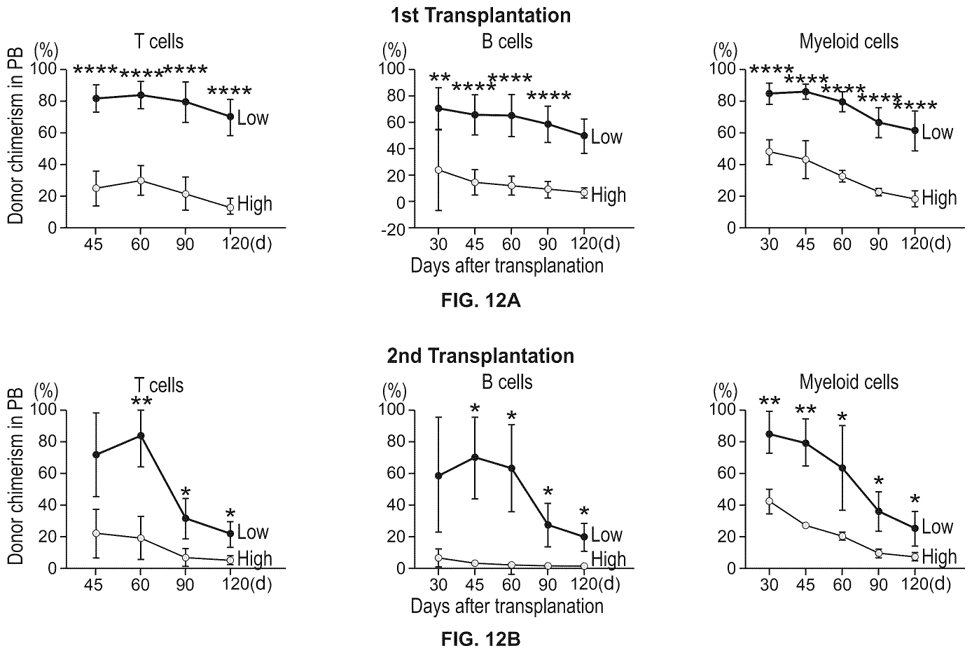

To directly compare the function between old CD150^{low} and CD150^{high} HSCs, serial competitive transplantation was performed (FIG. 3H). It was found that CD150^{low} old HSCs had significantly better engraftment capacity compared to their CD150^{high} counterparts in both the first and second transplantation (FIG. 3I and FIGS. 12A and 12B). These results demonstrate that CD150 marks transcriptome and epigenome heterogeneity; also shown is that the functional heterogeneity of old HSCs with CD150^{low} HSCs have better function than CD150^{high} HSCs.

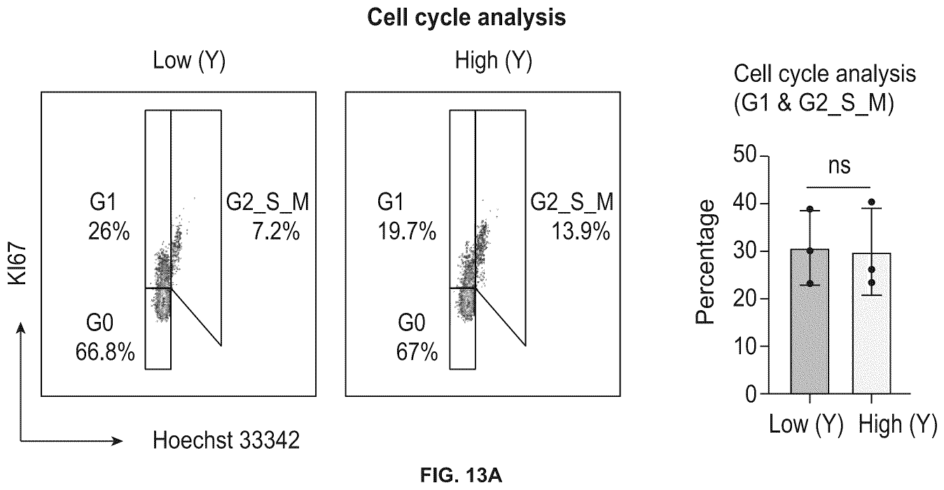

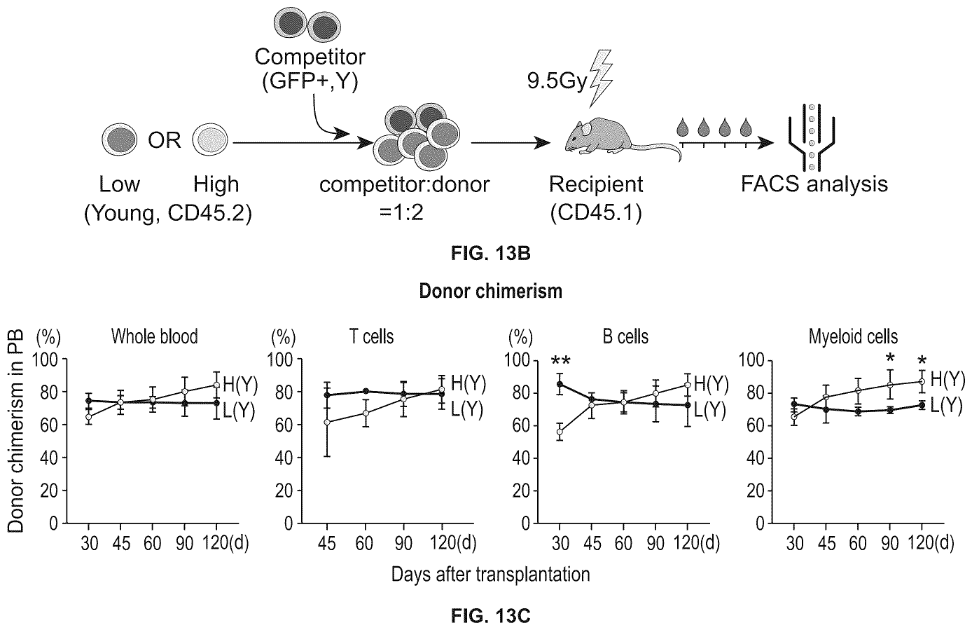

Although young HSCs exhibit a homogeneous transcriptome (FIG. 2D) and aging score (FIG. 2H), their CD150 levels are variable (FIG. 3B). This raised the question whether CD150^{low} HSCs are also functionally superior to CD150^{high} HSCs in young mice. To address this question, cell cycle was investigated and similar HSC transplantation experiments were performed using HSCs from young mice (FIG. 13A and FIG. 13B). Consistent with their transcriptome and aging score homogeneity, no significant functional difference was observed between CD150^{low} and CD150^{high} young HSCs for cell cycle analysis or peripheral blood (PB) chimerism during the period of 120 days after transplantation (FIG. 13A and FIG. 13C). However, a significantly higher contribution of CD150^{high} HSCs to myeloid cells was noted, along with an upward trend in PB chimerism when compared to CD150^{low} young HSCs. These results indicate that CD150^{high} HSCs from young mice have comparable or even better function than CD150^{low} HSCs, which is in contrast with that in old mice.

### Example 5: Old CD150^{high} HSCs are Defective in Differentiation, but not in Self-Renewal or Activation

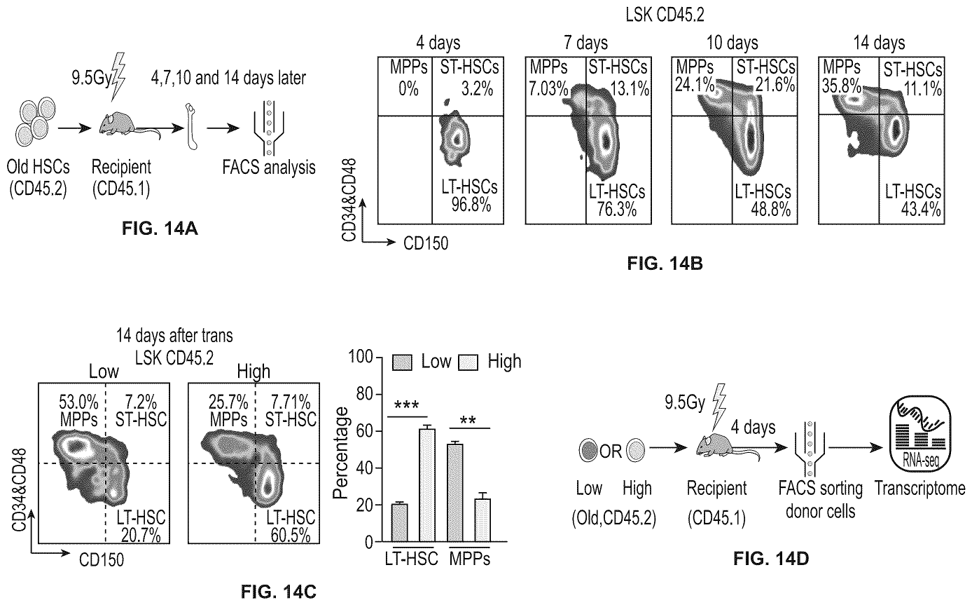

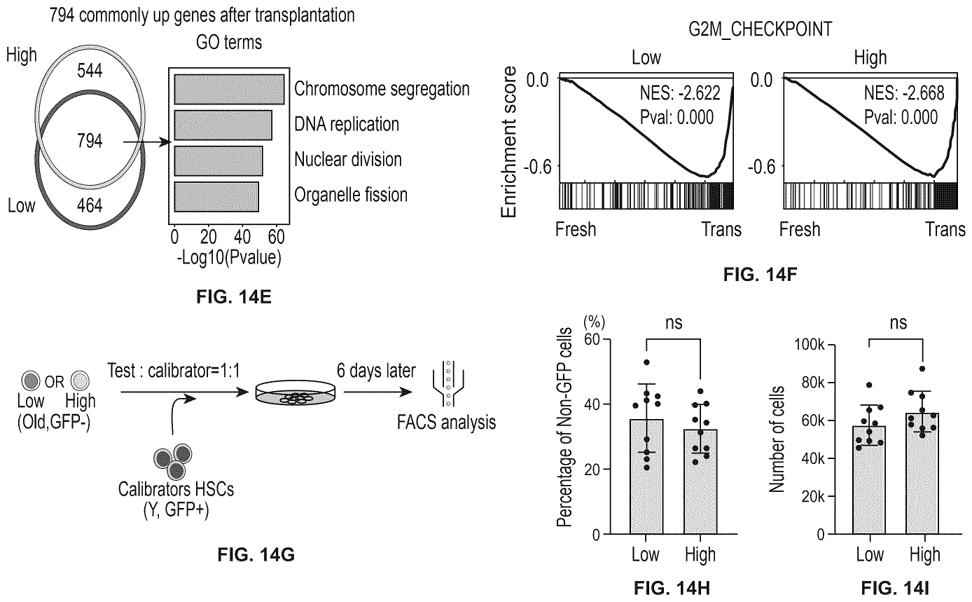

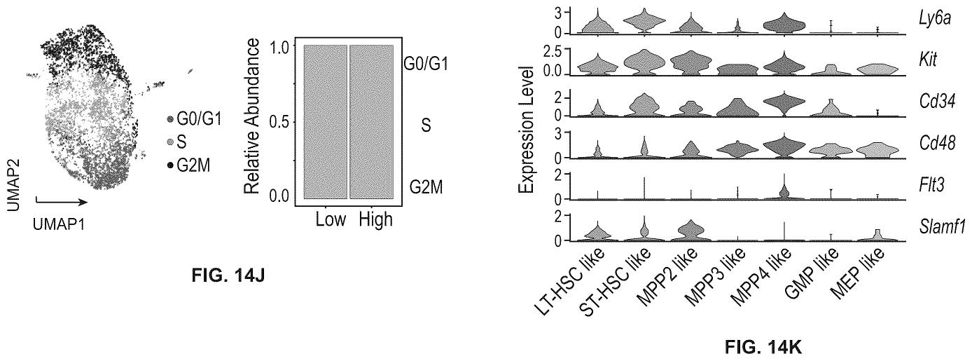

Experiments were conducted to dissect the mechanism underlying the differential repopulation capacity of CD150^{low} and CD150^{high} HSCs in old mice. Considering that the capabilities of self-renewal and differentiation are the two major features of stem cells, both features were compared after their transplantation. The differentiation dynamics were determined by analyzing the differentiation state of old HSCs at different time points after transplantation (FIG. 14A). It was found that transplanted old HSCs started to differentiate on day 7, and a progenitor subpopulation became apparent on day 10 (FIG. 14B). Based on this information, old CD150^{low} and CD150^{high} HSCs were separately transplanted, and the donor-derived hematopoietic stem and progenitor cells (HSPCs) were analyzed in bone marrow on days 7 and 14 after transplantation (FIG. 4A). Notably, it was found that CD150^{high} HSCs showed a significantly lower ratio in short term HSCs (ST-HSCs) and multipotent progenitors (MPPs), but a higher ratio in LT-HSCs compared to CD150^{low} HSCs (FIG. 4B and FIG. 14C), indicating that old CD150^{high} HSCs might have differentiation and/or activation (quiescent to activated state) defects compared to CD150^{low} HSCs.

To determine whether old CD150^{high} HSCs exhibited an activation defect, transcriptomes of CD150^{low} and CD150^{high} HSCs were profiled 4 days after transplantation, when the HSCs had yet to initiate differentiation, but were under stimulation (FIG. 14D). By comparing with freshly isolated HSCs (without activation), 794 commonly up-regulated genes in both CD150^{low} and CD150^{high} HSCs were identified after transplantation (FIG. 14E). The commonly up-regulated genes are highly enriched in terms related to cellular division. Consistently, the G2M checkpoint related genes were similarly upregulated in both CD150^{low} and CD150^{high} HSCs upon transplantation (FIG. 14F). These results indicate that old CD150^{high} HSCs can be effectively activated as CD150^{low} HSCs following transplantation. The proliferation capacity of old CD150^{low} and CD150^{high} HSCs in culture were also compared (FIG. 14G). It was found that old CD150^{low} and CD150^{high} HSCs exhibited comparable proliferation rates (FIG. 14H and FIG. 14I). Taken together, these results indicate that impaired repopulation capacity of old CD150^{high} HSCs is caused by defective differentiation, but not activation or proliferation.

To rule out a possible delay in differentiation of old CD150^{high} HSCs after transplantation, the persistence of the differentiation and self-renewal capacity of old CD150^{high} HSCs were analyzed over an extended period of 5 months. Old CD150^{low} or CD150^{high} HSCs (GFP negative) were co-transplanted with competitors (GFP positive) and the differentiation and chimerism of donor HSCs were analyzed in both bone marrow and PB 5 months later (FIG. 4C). A consistent differentiation defect of old CD150^{high} HSCs was observed when compared to their CD150^{low} counterparts when analyzing donor derived HSPCs in the bone marrow (FIG. 4D). Additionally, the donor chimerism of old CD150^{high} HSCs exhibited a clear trend of decline from LT-HSCs through ST-HSCs, MPPs, to PB, while the donor chimerism of old CD150^{low} HSCs remained relatively stable (FIG. 4E), supporting a long-term differentiation defect of old CD150^{high} HSCs. Collectively, these results indicate a long-lasting impairment in differentiation, but not in activation or self-renewal of old CD150^{high} HSCs, which explains the diminished repopulation capacity of this subpopulation.

### Example 6: Old CD150^{high} HSCs are Defective in the LT-HSCs to ST-HSCs Transition

To determine the specific stage when the differentiation defects of old CD150^{high} HSCs occur, comparative scRNA-seq analysis of the HSPCs derived from old CD150^{low} and CD150^{high} HSCs was performed 14 days after transplantation. 2,406 and 1,925 high quality HSPCs derived from transplanted CD150^{low} and CD150^{high} HSCs, respectively were obtained (FIG. 4F). Cell cycle phase analysis indicated that similar proportions of HSPCs derived from CD150^{low} and CD150^{high} HSCs were actively cycling (FIG. 14J), consistent with the conclusion that CD150^{low} and CD150^{high} HSCs can be similarly activated by transplantation (FIG. 14E and FIG. 14F).

Based on the expression of the HSPC marker genes and the HSPC sorting markers (Ly6a, Kit, Cd34, Cd48 and Slamf1), the 4,331 HSPC were divided into 7 subclusters that include LT-HSC-like, ST-HSC-like, MPP2-like, MPP3-like, MPP4-like, GMP-like and MEP-like cells (FIG. 4G and FIG. 14K). When HSPCs derived from CD150^{low} and CD150^{high} HSCs were compared, it was found that HSPCs derived from CD150^{high} HISCs were significantly enriched in LT-HSC-like cells compared to those derived from CD150^{low} HSCs (FIG. 4G and FIG. 4H), consistent with the FACS analysis (FIGS. 4B and 14C). Since the proportions of CD150^{low} and CD150^{high} HSCs derived ST-HSCs were comparable, the data indicated that the differentiation defects of CD150^{high} HSCs were mainly at the stages of LT-HSC to ST-HSC, and ST-HSC to MPP4 transitions. Given that MPP4 are the major progenitor cells of the lymphoid lineage, the substantial decrease in the MPP4 population might be a major cause of the biased differentiation of CD150^{high} HSCs. Collectively, comparative bone marrow scRNA-seq analysis of transplanted old HSCs indicated that the differentiation defects of old CD150^{high} HSCs is caused by a block of LT-HSCs to ST-HSCs transition.

### Example 7: The “Youthful” Subset of Old HSCs can Alleviate Aging Phenotypes and Extend Lifespan

Transplantation of young HSCs as an anti-aging therapy is impractical for humans due to immune incompatibility among different individuals. The demonstration that HSCs in old mice have molecularly and functionally “youthful” CD150^{low} HSCs prompted an investigation of whether this subset of HSCs can help to attenuate aging phenotypes of old mice. To this end, CD150^{low}, whole HSCs (un-selected), or CD150^{high} HSCs from old mice were transplanted into 13-month-old recipients and aging phenotypes were analyzed 5 months later (FIG. 5A). Compared to recipients that received whole-HSCs and CD150^{high} HSCs, mice transplanted with CD150^{low} HSCs showed a higher percentage of B cells, and a lower percentage of T cells and myeloid cells (FIG. 5B), as well as increased blood cell numbers, including lymphoid cells, neutrophils, eosinophils and red blood cells (FIG. 5C). Importantly, a significant increase in the naïve T cell ratio and a decrease in Tom and Tem ratios in the CD8⁺ (FIG. 5D) and CD4⁺ (FIG. 15A) T cells were also observed in mice that had received CD150^{low} HSCs relative to those that had received whole-HSCs or CD150^{high} HSCs. These results suggest that transplantation of old CD150^{low} HSCs can improve both hematopoiesis and immune parameters in old mice.

To determine whether improved hematopoiesis and immune parameters could benefit physical performance, a series of tests that measure muscle strength (grip strength, wire hanging), coordination (beam balance, pole test), and endurance (rotarod and treadmill) were performed. Consistently, a general trend of degrading physical performance in the order of CD150^{low}, whole-HSCs, and CD150^{high} HSCs (FIG. 5E) was found, without significant differences in their body weight (FIG. 15B). In almost all cases, mice transplanted with old CD150^{low} HSCs performed significantly better than those transplanted with CD150^{high} HSCs. Consistent with improved physical performance, it was found that old mice that had received CD150^{low} HSCs displayed a lower epigenetic age (FIG. 5F). Importantly, aged recipient mice transplanted with old CD150^{low} HSCs exhibited a substantial lifespan extension, achieving 9.2% and 11.7% increase in median lifespan compared to counterparts transplanted with whole-HSCs or CD150^{high} HSCs (FIG. 5G).

Taken together, these results indicate that not only transplantation of young HSCs, but the molecularly and functionally “youthful” subset of old HSCs can attenuate aging phenotypes of old mice. Considering that the functionally defective CD150^{high} HSCs increase with aging, these results are indicative that rejuvenation might be achieved by reducing or removing the functionally defective HSCs from old mice.

### Example 8: Reducing Dysfunctional HSCs can Alleviate Aging Phenotypes in Old Recipient Mice

To assess whether reducing defective HSCs in old mice can enhance hematopoiesis and mitigate aging-related phenotypes, a transplantation experiment was performed, as follows: 500 old CD150^{low} HSCs were co-transplanted into irradiated middle-aged (13 months) mice with 2,500 (1:5 ratio), 1,000 (1:2 ratio) and 0 (1:0 ratio) CD150^{high} old HSCs in order to mimic whole-HSC, partial removal, and complete removal of CD150^{high} HSCs, respectively. Five months after the transplantation, the hematopoietic system and physical capabilities of recipient mice were assessed to compare the aging status of the three groups (FIG. 6A). The 1:5 ratio served as a control, which was determined based on the percentage of q3 to the total HSC in old mice revealed by scRNA-seq (FIG. 2D).

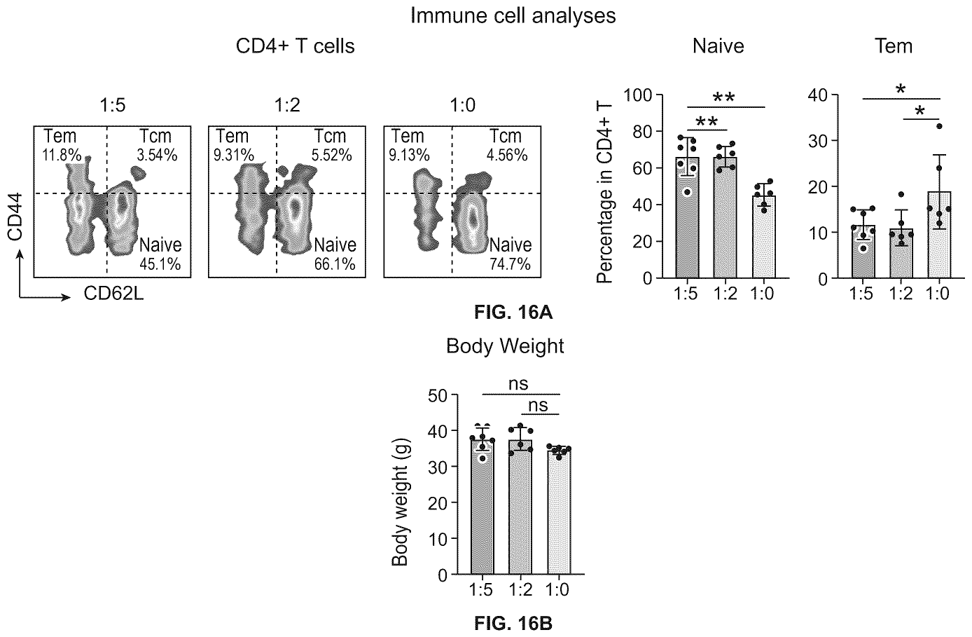

Peripheral blood (PB) analysis indicated that compared to the 1:5 group, the 1:2 and 1:0 groups had an increased percentage of B cells and a decreased percentage of myeloid cells (FIG. 6B). Complete blood counting analysis also showed higher numbers of whole white blood cells, neutrophils, and lymphoid cells in the 1:0 and 1:2 groups compared to the 1:5 group (FIG. 6C). Importantly, increased naïve CD4⁺ and CD8⁺ T cells, as well as decreased effective memory T cells, were also detected in the 1:2 and 1:0 groups compared to the 1:5 group (FIG. 6D and FIG. 16A), indicating the presence of the defective CD150^{high} HSCs had adverse effects on the “youthful” functional CD150^{low} HSCs for hematopoiesis and immune function in old mice.

Next, it was examined whether reducing the CD150^{high} HSCs ratio could improve physical functions of the recipient mice by performing a multitude of physical tests. The results indicated consistent improved physical functions in the 1:2 and 1:0 groups compared to the 1:5 group, including muscle strength (grip strength), motor coordination (pole test, beam balance, and rotarod), and locomotor activity (open field test), with no notable differences in body weight (FIG. 6E and FIG. 16B). These findings not only confirmed the adverse effect of the defective CD150^{high} HSCs in aged mice, but also suggested that their reduction in old mice can ameliorate aging-related phenotypes. It is noted that the 1:2 group exhibited a significantly younger and healthier hematopoietic system, immune parameters, and enhanced physical functions compared to those of the 1:5 group. This observation indicates that anti-aging effects can be accomplished by partial elimination of defective HSCs in aged mice, highlighting its application potential, since partial removal is much more practical than complete removal.

### Example 9: Materials and Methods

The following materials and methods relate to the experimental studies described in Examples 1-8 hereinabove.

### Mice

All experiments were conducted in accordance with the National Institute of Health Guide for Care and Use of Laboratory Animals and approved by the Institutional Animal Care and Use Committee (IACUC) of Boston Children's Hospital and Harvard Medical School. For bulk and single-cell RNA-seq, 2-3 months young and 22-24 months old C57BL/6 mice were used (Jackson Lab #00664). For HSC transplantation, 2-3 months old young (Jackson Lab #00664) or 2-3 months old GFP+young (Jackson Lab #006567) mice were used. For competitive transplantation, competitor HSCs were collected from 3-4 months GFP+young (Jackson Lab #006567) mice. Except as particularly mentioned, the recipient mice were B6 CD45.1 mice (Jackson Lab #002014), and the helper cells were also collected from B6 CD45.1 mice. For old recipient mice, 13 months old wild type (Jackson Lab #00664) were used. The helper cells were from age-matched wild type mice.

### Long Term Hematopoietic Stem Cell (LT-HSC) Sorting and Transplantation

Bone marrow (BM) cells were collected by crushing tibias, femurs, pelvic bone and spin (spin cord is removed). The resulting cell suspension was filtered through 70 μm cell strainers and red blood cell were lysed with red blood cell lysis buffer (ebioscience catalog #00-4333-57). To improve the sorting efficiency, filtered cells were further enriched by removing lineage positive cells. For detail, the filtered cells were first stained with biotin-conjugated antibodies against lineage markers (CD4, CD8, Gr-1, CD11b, CD5, B220 and Ter119). Lineage positive cells were removed by streptavidin-conjugated magnetic beads (STEMCELL catalog #19856), and the lineage negative cells were further stained with antibodies against mouse c-Kit, Sca-1, CD48, CD34, CD150. The dead cells were labeled with DAPI. The LT-HSCs were defined as Lin⁻Sca-1⁺c-Kit⁺CD48⁻CD34⁻CD150⁺. To prepare helper cells, bone marrow cells were collected from long bones (tibias and femurs) of CD54.1 mice (JACKSON LAB #2014) by flushing. After lysing red blood cells, all HSPCs were removed by Sca-1 positive selection kit (STEM CELL catalog #18756). The remaining cells were counted and used as helper cells for transplantation.

For transplantation experiments, a specific number of LT-HSCs were sorted with FACS sorter (SONY SH800). Sorted LT-HSCs were mixed with 3×10⁵ helper cells and transplanted into lethally irradiated (9.5 Gy) recipient mice (Jackson lab #2014) using 1 ml syringe attached with a 27.5-gauge needle.

Cell Cycle and DNA Damage Marker γH2AX Analysis of HSCs with FACS

The analysis of cell cycle status of HSCs was performed as previously described (Szade, K., et al., (2016), Methods Mol Biol, 1516, 91-99. 10.1007/7651_2016_361). Briefly, bone marrow cells were prepared, enriched, and stained with HSC marker antibodies. The enriched bone marrow cells were then fixed and permeabilized with BD Intra Kit (#641776). After washing, the fixed HSCs were stained using a monoclonal antibody against mouse KI67 (eBioscience, #11-5698-80) and Hoechst33342 (Thermo Scientific, #R37165) or using an antibody against DNA damage marker γH2AX (Cell Signaling Technology, #9719S). The cell cycle state of the HSCs or DNA damage marker γH2AX levels were analyzed with FACS after washing.

### Bulk RNA-Seq Library

A bulk-RNA sequencing library was constructed based on Smart-seq2 (Picelli, Björklund Å et al. 2013). SMART-Seq v4 Ultra Low Input RNA Kit for Sequencing (Takara catalog #634890) was used and experiments were performed according to the manufacturer's instructions. Briefly, sorted HSCs were incubated in lysis buffer with RNase inhibitor on dry ice for 1 minutes, and then thawed at room temperature. First-Strand Buffer, SMART-Seq v4 Oligonucleotide, 3′ SMART-Seq CDS primer II and RNase inhibitor were added into the lysed cells and incubated at 72° C. for 3 min before being put on ice. Incubation was performed after adding SMARTScribe II Reverse Transcriptase as follows: 42° C. for 90 minutes, then 70° C. for 10 minutes. Without purification, SeqAmp PCR Buffer, PCR Primer II A and SeqAmp DNA Polymerase were added for pre-amplification using thermal cycler for 12 cycles following the manufacturer's instructions. The amplified cDNA was then purified by 1.8× volume of SPRIselect beads (Beckman Coulter catalog #B23318). Next, 200 μg cDNA was used for library preparation with Nextera XT DNA Library Preparation Kit (Illumina catalog #FC-131-1024) according to the manufacturer's instructions.

scRNA-Seq Library Preparation

Single-cell RNA seq library was prepared with Chromium Next GEM Single Cell 3′ Reagent Kits V3.1 (Dual Index) from 10× Genomics according to the manufacturer's instructions. Briefly, single HSCs were sorted by FACS into PBS solution. Then microfluids were used to construct droplets containing single-cell, GEMs and reverse transcription reagents. 100 μl of droplets were incubated at 53° C. for 45 minutes, then at 85° C. for 5 minutes. Droplets were then released by Recovery Agent, cleaned up with Dynabeads MyOne SILANE and amplified by PCR for 12 cycles. The amplified cDNA was used for library construction according to the manufacturer's instructions.

### Peripheral Blood Analysis Via FACS

For PB analysis, up to 30 μL vein blood was collected from retro orbital sinus or tail tips into EDTA-coated tubes. Red blood cells were firstly removed by red blood cell lysis buffer (ebioscience catalog #00-4333-57). The remaining white blood cells were stained with mixed monoclonal conjugated antibodies (CD45.1, CD45.2, CD3, B220, Gr-1 and CD11b). After incubation in 4° C. and darkness for 30 minutes, PB was analyzed by FACS (BD FICR canto-II).

### HSC Culture In Vitro

Sorted LT-HSCs were cultured in STEMSPAN™ SFEM II (STEM CELL catalog #09605) with 10 ng/ml SCF (PeproTech, catalog #AF-250-03), 100 ng/ml TPO (PeproTech, catalog #315-14), 25 ng/ml Flt3L (PeproTech, catalog #250-31L) and 25 ng/ml IL3 (PeproTech, catalog #213-13). SCF (Stem Cell Factor) is a hematopoietic growth factor that exerts its activity by signaling through the c-Kit receptor. SCF and c-Kit are essential for the survival, proliferation and differentiation of hematopoietic cells committed to the melanocyte and germ cell lineages. Human SCF manifests low activity on murine cells, while murine and rat SCF are fully active on human cells. TPO (thrombopoietin) is a lineage-specific growth factor produced in the liver, kidney and skeletal muscle. It stimulates the proliferation and maturation of megakaryocytes, and promotes increased circulating levels of platelets in vivo. TPO signals through the c-mpl receptor, and acts as an important regulator of circulating platelets. Human and murine TPO exhibit cross-species reactivity. Six days after culture, the number of HSCs were quantified by FACS (BD FICR canto-II).

### Complete Blood Counting Analysis of Recipient Mice

For whole mouse blood analysis, 75 μL vein blood was collected from the retro orbital sinus and mixed with 225 μL 5 mM EDTA (Research Products International, catalog #E14000-250.0). Freshly collected whole blood was analyzed by Hematology system (ADVIA 120) 2 hours after collection.

### Naïve T, Tcm and Tem Analysis

The ratios of Naïve T cells, Tem and Tem were analyzed based on a previous report (Pakpour, N. et al. 2008, J Immunol, 180(12): 8299-8305). Briefly, PB was collected and red blood cells were removed by red blood cell lysis buffer (ebioscience catalog #00-4333-57). The remaining cells were stained with antibodies directed against CD45.1, CD45.2, CD4, CD8, CD44 and CD62L. T cell subtypes were defined as follows: naïve (CD62L^{high} CD44^{low}), Tcm (CD62L^{high} CD44^{high}) and Tem (CD62L^{low} CD44^{high}).

### Reduced-Representation Bisulfite Sequencing (RRBS) Library Preparation for HSCs

FACS sorted HSCs were lysed at 50° C. for 3 hours and 75° C. for 30 min in a 5 μl lysis buffer (20 mM tris-EDTA (Sigma catalog #T9285-100ML), 20 mM KCl (Thermo Scientific, catalog #AM9640G), 0.3% Triton X-100 (Sigma catalog #93443-100ML), and protease (1 mg/ml; Qiagen catalog #19155)). The lysed cells were then subjected to Msp I digestion at 37° C. for 3 hours and at 80° C. for 20 minutes. Following enzymatic digestion, end-repair mix (1 μl Klenow frag exo (5 U/μl), 0.2 μl of 10× TangoBuf, and 0.8 μl of dNTP mix) was added and incubated at 37° C. for 40 minutes and at 75° C. for 15 minutes. For adaptor ligation, the reaction was incubated at 16° C. for 30 minutes, 4° C. overnight, and at 65° C. for 20 minutes after adding the ligation mix (2.25 μl of H₂O, 1 μl of T4 ligase (30 U/μl); Thermo Scientific, catalog #EL0013), 0.5 μl of 100 mM ATP (Thermo Scientific, catalog #R0441), and 0.75 μM methylated adaptor). Bisulfite conversion reaction was then performed by the EpiTect Bisulfite Kit (Qiagen catalog #59104) following the manufacturer's instructions. Adapter-tagged BS-DNA was then amplified by 16 cycles using Kapa HiFi Uracil+Ready Mix (Kapa Biosystems, catalog #KK2801) with NEBNext Multiplex Oligos for Illumina (New England BioLabs catalog #E7500L).

### ATAC-Seq Library Construction

About 500-1000 cells were sorted by FACS in 10 μl tagmentation buffer (33 mM Tris-Acetate, 66 mM K-Acetate, 10 mM Mg-Acetate, 16% Dimethylformamide, and 0.02% Digitonin). 0.5 μl Tn5-Adapter complex (Diagenode) was added after FACS sorting and the samples were incubated at 37° C. for 30 minutes. Then, the tagmentation reaction was stopped by adding 10 μl stop buffer (100 mM Tris-HCl pH 8.0, 100 mM NaCl, 0.4% SDS, 40 μg/ml Proteinase K), and the samples were incubated at 55° C. overnight to release the tagmentation fragments. SDS was then quenched by adding 5 μl 25% Tween-20, and the samples were incubated on ice for 10 minutes. Next, 25 μl samples were mixed with 30 μl NEBNext High-Fidelity 2×PCR Master Mix (NEB), 2.5 μl P5 primer (10 μM) and 2.5 μl P7 primer (10 μM) for PCR amplification. The PCR products were then purified with SPRI beads with 1:1.6 ratio (96 μl beads) before sequencing.

### Physical Function Tests

For the grip strength test, a mouse was gently placed on the top of a grid strength meter (Bioseb, Model GT3) so that only its forepaws can grasp the grid. The tail of the mouse was pulled three times, and then the animal was returned to the cage for at least a 1 minute rest. Two (2) series of pulls were performed; the top 3 animals' maximum grip strength were averaged, and body weight was normalized for each mouse.

For the beam balance test, home-made equipment was used. Briefly, a 1-meter-long smooth wood strip was elevated to 50 cm tall from the table. A black box (20×20×20 cm) was put on the plate at one end and the mouse was put at the other end. For training day, a thicker pole was used (28 mm diameter). Each mouse was trained until it was able to go through the pole and to the box without dropping. The next day, the mice were tested with a thicker pole or a thinner pole (17 mm diameter), 3 times per mouse. Before being used for another test, the pole and box were cleaned with 70% ethanol. The latent time for the mice to go through the pole was recorded.

For the pole test, mice were placed at the top of a 50 cm grooved metal pole with a diameter of 1 cm with the head of the mice pointing down. The time from initial placement on the top of the pole to the time a mouse reached the base of the pole (forelimb contact with platform) was recorded with a stopwatch. Following a 30-minute rest in the home cage, the trial was repeated another 2 times. The average time was calculated as the latent time to go down for each mouse.

The rotarod test was used to evaluate motor coordination and balance. Mice were placed in separate lanes on the rod rotating at an initial speed of 4 rpm/min in an accelerating Rotarod system (Ugo Basile Apparatus). On the first day, each mouse finished a training program at speed of 4 rpm/min for 5 min and repeated another 2 times with a 20 min interval. For the test day, the apparatus was set to accelerate from 4 to 40 rpm/min in 5 min. The time and speed were recorded when the mice dropped or after two consecutive rounds of passive rotation. A test was performed one- or two-times per day for each mouse with an interval of at least 30 min. The test was continued on the next day, and last for 3 tests in total. The apparatus was cleaned between tests of the mice to make sure it was dry and clean. Results were the average over 3 tests.

For the wire hanging test, the mice were placed on a medal salamander with a 1-cm square mesh 50 cm above a cushioned surface covered with approximately 3-cm thick bedding. First, the mice were put on the salamander and then the salamander was inverted slowly. The stopwatch was started when the mouse was attached upside down. The latent time for the mouse to fall was recorded. Within a 30 min interval, each mouse was tested for 3 times and the results were the average over 3 trials.

For the treadmill fatigue test, the protocol was adapted and modified from a previous publication (Dougherty, J. P. et al., (2016), J Vis Exp., 10.3791/54052). Briefly, mice were acclimatized to the treadmill (Columbus Instruments Exer 3/6 Treadmill) for 2 consecutive days prior to testing. The fatigue zone was set as one mouse body-length from the shock grid. On day 1, the mice were placed on the treadmill and allowed to explore for 5 minutes (min) with the shock grid turned off. After 5 min, the shock grid was powered on at 1.5 mA, 3 Hz and the speed was set to 6 m/min for 5 min. After 5 min, the speed of 2 m/min was increased for every 2 min. The treadmill was stopped when the mice had run at 10 m/min for 5 min. On day 2, the same training program was performed, except that the top speed was 12 m/min for 5 min. On the test day, the speed was increased stepwise over time, up to maximum of 26 m/min. The fatigue standard was met when mice were present in the fatigue zone for more than 5 seconds in three separate instances. The latency time for fatigue of each mouse was recorded for analysis.

For the fear conditioning test, contextual and cued fear conditioning (CCFC) was implemented. In brief, mice underwent the following training protocol on the first day: 2 minutes of acclimation, followed by a 30-second tone, then a 2-second foot shock (0.5 mA). This was followed by a 2-minute intertrial interval (ITI). Subsequently, another 30-second tone was paired with a 2-second footshock (0.5 mA), and then there was a 1-minute period before lights out. The following day, a context test was conducted where mice were placed in the same context for 5 minutes without any shock or tone. On the third day, mice were assessed in a modified context: 3 minutes of acclimation without tone, followed by 3 minutes with tone. Video was captured using Noduls MediaRecorder (version 4.0), and freezing behavior was analyzed using Noldus Etho Vision XT17.

For the Novel Y-Maze test, the protocol was adapted from previous study (Chen, R., et al. (2022), Sci Adv, 8, eabm1077. 10.1126/sciadv.abm1077). Briefly, mice were pre-acclimated to a separate holding room for 30 minutes before testing. The Y-maze procedure consisted of a 3-minute habituation phase, followed by a 3-minute test phase, with a strict 2-minute ITI. In the habituation phase, one arm (left or right) was obstructed, and the start arm remained consistent for each mouse. After habituation, mice were briefly returned to their holding cage during the ITI. The obstruction was then removed, and the maze was cleaned before the test phase. Then, the mouse was placed back into the start arm for the test trial and returned to the home cage after the trial was completed. Distance and time traveled in the maze were recorded and analyzed with Noldus Etho Vision XT17.

For the open field test, the protocol was adapted from previous study (Chen, R. et al., (2022), Sci Adv, 8, eabm1077. 10.1126/sciadv.abm1077). In the open-field test, the apparatus was a transparent enclosure with a base measuring 27.3 cm by 27.3 cm and walls with a height of 20.3 cm. The central zone accounted for half of the total area. Prior to testing, mice were acclimated to the test environment for a minimum of 20 minutes. Each mouse was then positioned in the center of the arena to begin the assessment. Movements were tracked for a duration of 20 minutes and data were collected in intervals of 5 minutes using a tracking system (Med Associates, St. Albans, VT, ENV-510). Analysis included standard locomotor activity parameters, such as total distance moved, average speed, active and inactive periods, as well as vertical events in the first 10 minutes.

### RNA-Seq Data Analysis

For bulk RNA-seq datasets, adaptor of all sequenced reads was first trimmed by Trim Galore with parameter “-illumina-paried”. The filtered reads were then aligned to the mm10 reference genome by Hisat2 (version 2.0.0-b)⁷³ with parameters “--no-mixed --no-discordant --dta-cufflinks --no-unal”. The aligned SAM files were then converted to BAM files with Samtools (version 1.14) for further analysis. Next, HTSeq-count (V0.12.4) was used to calculate the count of reads for each gene, with the main parameters set as: -f bam -s no -r pos. The reference genome was downloaded from the USCS Table Browser. To quantify gene expression, the read count for each gene was normalized using DESeq2 (V1.22.2). The differentially expressed genes from each group were filtered with following criteria: adjusted P-value <0.05 and fold-change >2 or <0.5.

### ATAC-Seq Analysis

For ATAC-seq datasets, sequencing adaptors and low-quality reads were removed by Trim Galore with parameters: -q 20 --length 30 --paired. Then the clean reads were mapped to mouse reference genome (mm10) by bowtie2 (Version 2.5.1) with parameter: “—N 1-L 25 -X 2000 -no-discordant” (ATAC-seq). After converting aligned SAM to BAM files using Samtools (Version 1.13), Picard (Version 2.26.4) was used for removing PCR duplications. Next, read counts were normalized by Reads Per Kilobase per Million mapped reads (RPKM) and converted to bigwig via bamCoverage (Version 3.5.1). The peaks were called by MACS2 (Version 2.2.7.1) with parameter “-t file -f BED -g mm -outdir OUTPUT -n NAME --nomodel --nolambda” (ATAC-seq). The “norrowpeak” files were used for downstream analysis. The peaks that overlap in more than 60% of the replicate samples in each group were kept for feature counts and subsequent analysis using DiffBind (V3.0.15). The read counts for each peak were normalized using DESeq2 (V1.30.1). Peaks that differed significantly between groups were identified using DESeq2 with the following criteria: P-value <0.05 and fold-change >1.5 or <0.67. Deeptools (Version 3.5.1) was used to visualize the read coverages at peak regions.

scRNA-Seq Data Analysis

Paired reads from single-cell RNA seq were first aligned to mm10 reference genome with Cellranger --count (version 3.0.2) from 10× Genomics. Transcriptome annotation composed of protein coding genes was download and processed from Ensembl released-102. To eliminate low-quality single cells, single cells were filtered out with feature numbers below 800 for freshly isolated HSCs (FIGS. 2A-2H) and below 2,000 for short-term transplanted HSCs (FIGS. 4A-4H). Additionally, cells with mitochondrial transcripts accounting for more than 5 percent were also removed. Then, the remaining single cells were processed and analyzed with Seurat (V4.0.2) (Hao, Y. et al., (2021), Cell, 184(13): 3573-3587.e3529). The young and old single cell data was first merged and then normalized and scaled. Two thousand variable feature genes were identified and used for UMAP presentation.

### Epigenetic Age Calculation

Paired fastq files from RRBS seq were first merged and processed as a single file. Adaptors of all sequenced reads were trimmed by Trim Galore with parameters --Illumina. Adaptor trimmed reads were mapped using Bismark (v0.20.0) (Krueger F. et al. 2011, Bioinformatics 27(11): 1571-1572) using parameters --fastq -L 30 -N 1 --non_directional. Methylated CpGs were interpreted by bismark_methylation_extractor with parameters -s --no_overlap --report --bedGraph. For base resolution 5mC level calculation, a cutoff of minimal 3× coverage was required for each CpG site. The 5mC level for certain CpG was calculated using the number of methylated CpGs to divide the number of total CpGs detected on the CpG site. Epigenetic age was calculated based on the identified sites from a previous study (Meer, M. V. et al. 2018, Elife, 7).

### Data Accession

All data have been deposited to GEO with the accession number GSE233879. Access to the data by the reviewers can be achieved with the link below using token “ynadskqcxvmdbal”: worldwideweb.ncbi.nlm.nih.gov/geo/query/acc,cgi?acc-GSE233879.

### Example 10: Targeted Depletion of Dysfunctional CD150^{high} HSCs In Vitro

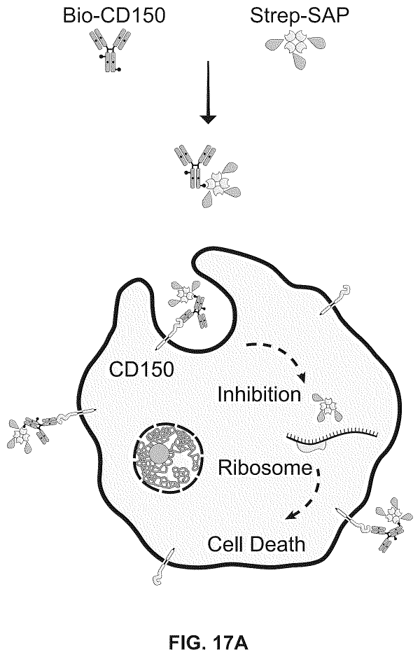

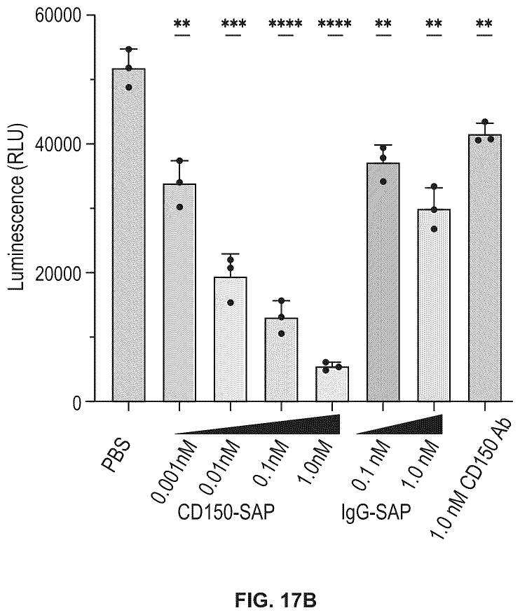

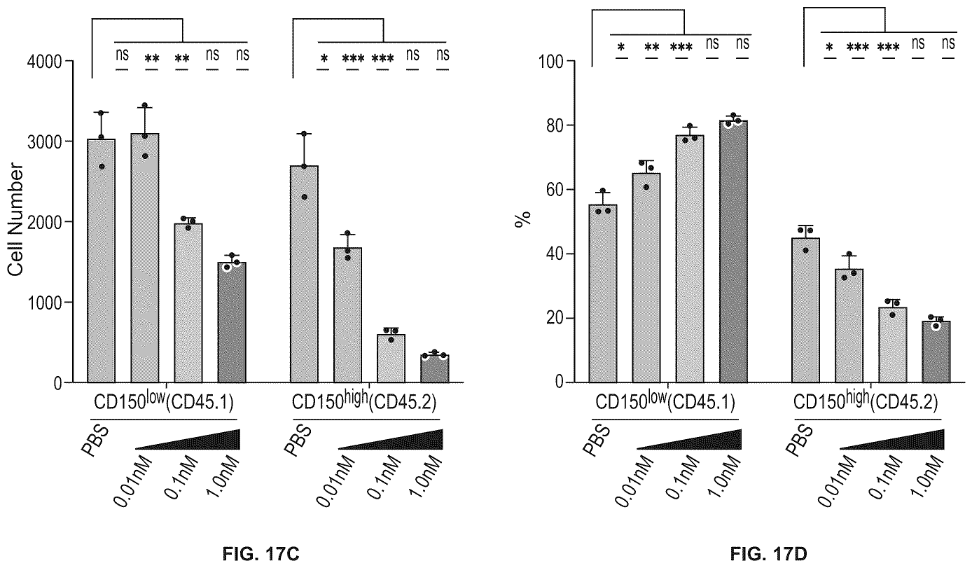

To specially remove dysfunctional CD150^{high} HSCs from a sample, an antibody-toxin conjugation method was employed according to previous methods (Palchaudhuri R. et al., (2016), Nature biotechnology 2016; 34(7): 738-45; Czechowicz A. et al., (2019), Nat Commun; 10(1): 617), (FIGS. 17A-17D). In this system, through biotin and streptavidin binding, CD150 antibody is conjugated with saporin, a specific toxin that inhibits translocation of the nascent protein chain during protein synthesis. (Polito L., et al., (2013), Toxins (Basel), 5(10): 1698-722). When the biotin-conjugated CD150 antibody is complexed with streptavidin-conjugated saporin (CD150-SAP), it is expected to induce cell death upon endocytosis into the targeted cells. The killing effect of the CD150-SAP was assessed using HSCs in vitro. To this end, purified old HSCs (e.g., obtained from old mice (about 24 months) were treated with different concentrations of CD150-SAP, IgG-SAP, or CD150 Ab for 3 days, and live cells were quantified by the ATP concentration using a titer assay. The results showed that CD150-SAP treatment resulted in HSC killing in a dose-dependent manner (FIG. 17B). Old or aged mice exhibit HSC heterogeneity as they contain CD150^{high} HSCs and CD150^{low} HSCs, which have functional differences as described herein. Based on the FACS profile of HSCs analyzed in the sample used in the experiments described in this example, the CD150^{high} HSCs represented the top 15% of HSCs and the CD150^{low} HSCs represented the bottom 15% of HSCs.

It was found that the IgG-SAP control reduced the cell number compared to a PBS control, which indicated a certain level of off-target effect. Thus, the killing specificity of CD150-SAP under a defined concentration should be carefully evaluated. To this end, CD150^{low} HSCs from a 17-month-old CD45.1 mouse were mixed with the same number of CD150^{high} HSCs from an age-matched CD45.2 mouse, and the cell numbers and percentages of CD150^{low} HSCs (CD45.1⁺) and CD150^{high} HSCs (CD45.2⁺) were analyzed 3 days after the treatment with different concentrations of CD150-SAP.

It was found that with increasing CD150-SAP concentration, the numbers of both CD150^{low} and CD150^{high} HSCs were decreased when compared to the PBS treated control (FIG. 17C). However, at the lowest concentration of 0.01 nM, CD150-SAP significantly reduced the number of CD150^{high} HSCs, while no obvious effect was observed for the CD150^{low} HSCs (FIG. 17C). These data indicate that at a concentration of 0.01 nM, the CD150-SAP antibody conjugate exhibited a specific killing effect on CD150^{high} HSCs. In addition, the decreased ratio of CD150^{high} to CD150^{low} HSCs in response to increased CD150-SAP also supports that the CD150^{high} HSCs were preferentially targeted by CD150-SAP (FIG. 17D).

### Example 11: Targeted Depletion of the Dysfunctional CD150^{high} HSCs In Vivo

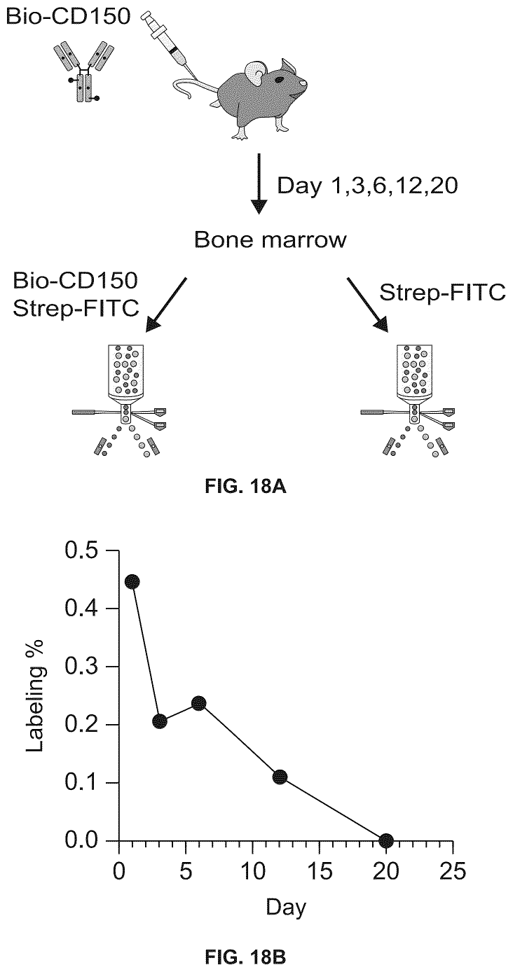

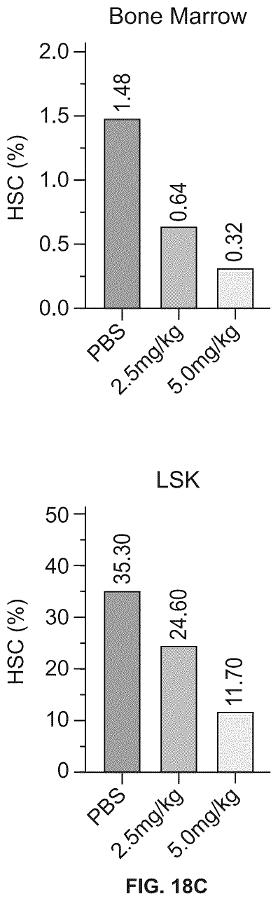

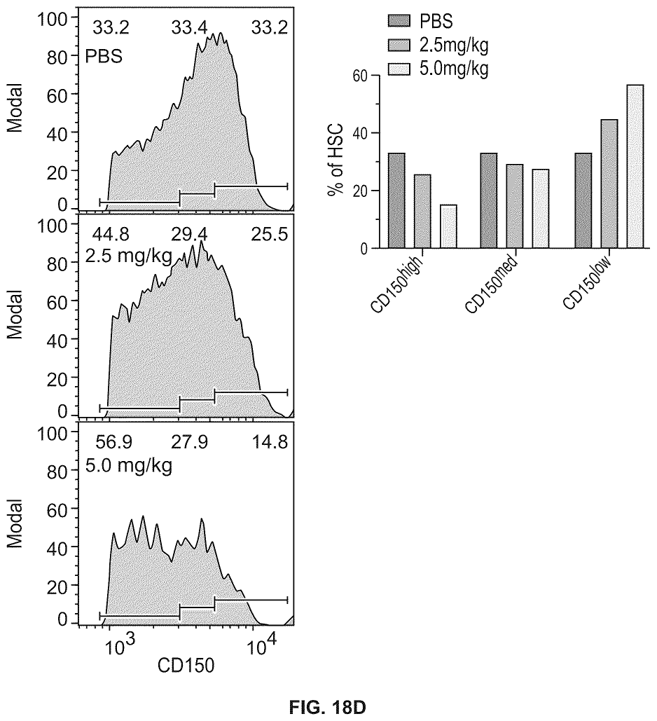

Having demonstrated the preferential killing effect of CD150-SAP toward CD150^{high} HSCs in vitro, it was investigated whether the effect was also observed in vivo. Because CD150 is one of the surface markers of HSCs for FACS analysis, the injected CD150-SAP complex could hinder the flow detection of CD150⁺ cells after CD150-SAP treatment. To avoid such a potential masking effect, a time course experiment was performed to measure the persistence of the CD150 antibody after injection of the CD150-SAP complex (FIG. 18A). To this end, mice were injected with bio-CD150 antibody, and bone marrow was collected on different days and divided into two parts. One part was used to determine the percentage of total HSCs that can be labeled by added bio-CD150 antibody, while the other part received no additional bio-CD150 antibody to reflect the labeling of HSCs by the injected bio-CD150 antibody. The labeling efficiency of HSCs by bio-CD150 antibody was calculated by comparing these two percentages at different time points. The results indicated that the percentage of labeled HSCs gradually decreased, reaching zero by day 20 (FIG. 18B), indicating that the injected bio-CD150 antibody was totally removed and absent by this time point. Consequently, 3 weeks was established as the minimum time needed to use FACS to measure the CD150-SAP killing effect.

To examine whether CD150-SAP can deplete CD150^{high} HSCs in vivo, two doses of CD150-SAP were injected into 17-month-old C57BL/6J mice intravenously compared with PBS injection as a control. The percentage of HSCs in the total bone marrow cells or LSK was calculated 3 weeks after the treatment, which revealed a dose-dependent decrease when compared with the PBS control (FIG. 18C), indicating that HSCs were at least partially depleted by the treatment. To assess whether CD150^{high} HISCs were preferentially depleted by the treatment, the distribution of HSCs in relation to their CD150 level was analyzed 3 weeks after the treatment. The results indicated a dose-dependent shift of the CD150^{high} to the CD150^{low} population (FIG. 18D), peak shift to the left). Quantification of the distribution of the HSCs indicated that the treatment resulted in a decrease in the percentage of CD150^{high} HSCs concomitant with an increase in the CD150^{low} HSCs when compared with the control (FIG. 18D). These data support the finding that CD150-SAP preferentially depleted CD150^{high} HSCs in vivo.

### Example 12: Depletion of Dysfunctional CD150^{high} HSCs In Vivo Improves Hematopoiesis

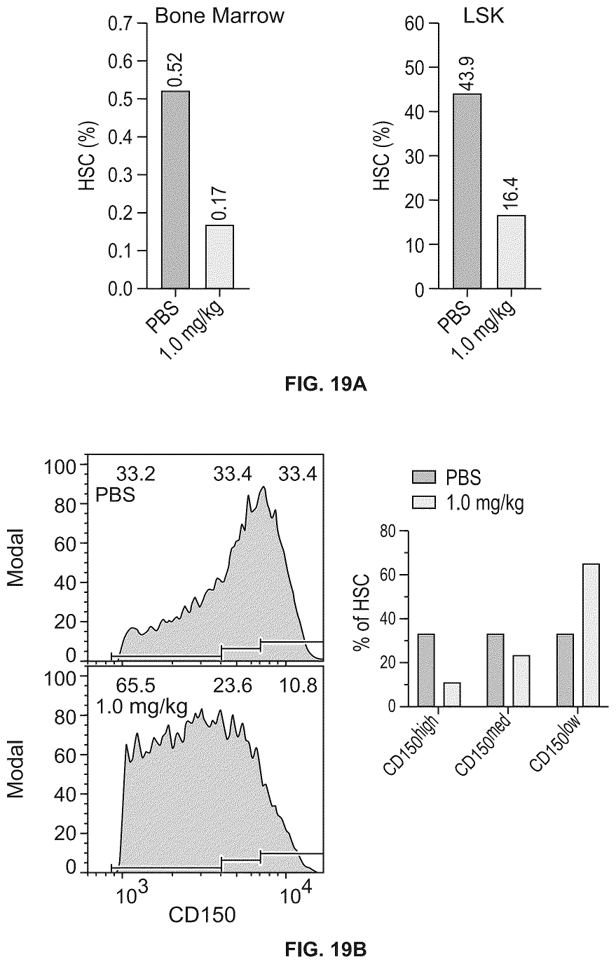

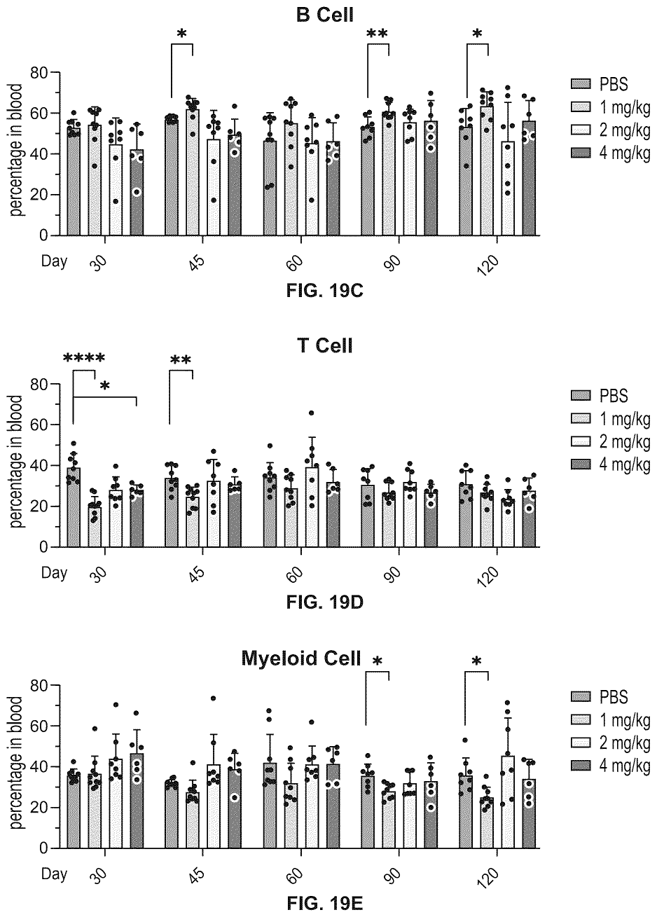

To avoid potential off-target depletion, a lower dosage (1.0 mg/kg) of CD150-SAP was also evaluated. Forty-five (45) days after the injection, the percentage of HSCs was determined in bone marrow and LSK. It was found that at a dosage of 1.0 mg/kg, CD150-SAP treatment was still able to reduce about 60% of the HSCs compared with control (FIG. 19A). Importantly, compared to the PBS-injected control, the distribution pattern of CD150 expression level on HSCs shifted to the left side (FIG. 19B, left), leading to a decreased ratio of CD150^{high} to CD150^{low} HSCs in old mice (FIG. 19B, right).

Because doses of CD150-SAP from 1 to 5 mg/kg all were able to decrease the CD150^{high} to CD150^{low} HSC ratio, experiments were performed to assess a dose that has a beneficial effect. In vivo experiments were performed by injecting three different doses (1, 2, 4 mg/kg) of CD150-SAP into 15-months old mice with PBS as a control. To monitor the effect of these treatments on hematopoiesis, peripheral blood analyses were performed at different time points after the treatment. It was found that treatment at 1.0 mg/kg CD150-SAP significantly increased the B cell percentage at days 45, 90, and 120 after the treatment, and decreased the myeloid cell percentage at days 90 and 120 after the treatment (FIG. 19C, FIG. 19E). The decreased T cell percentage at 30 and 45 days after the treatment might be due to the depletion of T cells in the blood, as some T cells also express CD150 (FIG. 19D). These results indicate that depletion of the dysfunctional CD150^{high} HSCs can at least partly correct the myeloid biased differentiation observed in old mice.

Consistent with the experimental results described herein, depletion of myeloid-biased HSCs (marked by NEO1 in most cases) using an antibody cocktail was reported to restore immune function in old mice (Ross J. B. et al., (2024), Nature; 628(8006): 162-70). This approach of simultaneous targeting both myeloid-biased HSCs and c-Kit positive progentior cells may cause serious side-effects and may not lead to a systemic body rejuvenation. It is noted that the killing mechanism as described and exemplified by experimental results herein is distinct and differs from the approaches of others. By way of example, according to the report of Ross et al., the myeloid biased HSCs are eliminated by phagocytosis facilitated by blocking CD47 signaling by continuous injection of high-dose CD47 antibodies into old mice. In contrast, the examples as described herein employ the ribosome toxin saporin to inhibit protein synthesis, thus providing an approach that preferentially eliminates the dysfunctional CD150^{high} HSCs in old mice and leaves the functional CD150^{low} HSCs largely unaffected when the appropriate concentration of the CD150-SAP complex is used. Notably, the amount of CD150 antibody used in the approach as described and exemplified herein is at least ten times lower than the amount of antibody reported by Ross et al. Given the conserved expansion of the dysfunctional HSCs in both mice and humans as subjects, the results of the studies described and exemplified herein provide an advantageous foundation for translational applicability in humans.

### Example 13: Material and Methods Related to Examples 10-12

### Mice

All experiments were conducted in accordance with the National Institute of Health Guide for Care and Use of Laboratory Animals and approved by the Institutional Animal Care and Use Committee (IACUC) of Boston Children's Hospital and Harvard Medical School. For in vitro CD150-SAP experiments, 18-20-month-old C57BL/6 mice (Jackson Lab #000664) and B6.SJL-Ptprcª Pepcb/BoyJ mice (Jackson Lab #002014) were used. For bio-CD150 labeling experiment, 8-week-old C57BL/6 mice (Jackson Lab #000664) were used. For in vivo CD150-SAP depletion experiments, 15-month-old C57BL/6 mice (Jackson Lab #000664) were used.

### Antibody-Saporin Conjugation and Administration

Bio-CD150 (Biolegend, clone TC15-12F12.2, catalog #B115908) or bio-IgG (BioLegend, clone RTK2758, catalog #400504) were desalted using ZEBA™ Spin Desalting Columns (Thermo Scientific, catalog #89882). Bio-CD150 or bio-IgG antibody was mixed with strep-SAP (Advanced Targeting Systems, catalog #IT-27-1000) in a 1:1 molar ratio and then diluted in PBS to the desired concentration. CD150-SAP was added to HSC culture medium or administered in 300 μL via tail vein intravenous injection.

### In Vitro Cell Viability Assay

HSCs were purified and cultured as previously described (Wilkinson A. C. et al., (2020), Nat Protoc; 15(2): 628-48). HSCs were plated in 96-well microtiter plates with 300-500 cells/well in a 100 μL volume of cell culture medium containing various concentrations of antibody-saporin-conjugate. After 72 hours, cell viability was determined using a Cell Titer assay (Promega, catalog #G9241).

### HSC Analysis

Bone marrow (BM) cells were collected by crushing tibias, femurs, pelvic bone and spine (spinal cord was removed). The resulting cell suspension was filtered through 70 μm cell strainers, and red blood cells were lysed with red blood cell lysis buffer (ebioscience, catalog #00-4333-57). To improve the sorting efficiency, filtered cells were further enriched by removing lineage positive cells. In particular, the filtered cells were first stained with biotin-conjugated antibodies against lineage markers (CD4, CD8, Gr-1, CD11b, CD5, B220 and Ter119). Lineage positive cells were removed by streptavidin-conjugated magnetic beads (STEMCELL, catalog #19856), and the lineage negative cells were further stained with antibodies against mouse c-Kit, Sca-1, CD48, CD34, CD150. The dead cells were labeled with DAPI. The samples were analyzed by FACS (BD FICR canto-II). HSCs are defined cells that express biomarkers as follows: Lin−Sca-1+c-Kit+CD48−CD34−CD150+.

### Peripheral Blood Analysis

Up to 30 μL vein blood was collected from retro-orbital sinus or tail tips into EDTA-coated tubes. Red blood cells were first removed by red blood cell lysis buffer (ebioscience, catalog #00-4333-57). The remaining white blood cells were stained with mixed monoclonal conjugated antibodies (antibodies directed against biomarkers CD45.1, CD45.2, CD3, B220, Gr-1 and CD11b) in FACS staining buffer (PBS containing 1% FBS and 1 mM EDTA). After incubation at 4° C. in the dark for 30 minutes, samples were washed with FACS staining buffer and resuspended with staining buffer containing 1 μg/ml DAPI. Peripheral blood samples were analyzed by FACS (pBD FICR canto-II).

### OTHER EMBODIMENTS

From the foregoing description, it will be apparent that variations and modifications may be made to the aspects and embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### TABLES 1A, 1B, 2, 3A, 3B

TABLE 1A
List of DEGs between young and old HSCs derived from bulk RNA-seq
Up-regulated genes in old HSCs when compared with young HSCs

| Genes | baseMean | log2FoldChange | IfcSE | stat | pvalue | pad |
|---|---|---|---|---|---|---|
| Ntf3 | 91.46564939 | 9.937234709 | 1.323283145 | 7.509530175 | 5.93399E−14 | 1.56581E−11 |
| Mab21l2 | 159.909083 | 9.779494394 | 1.264906102 | 7.73139949 | 1.06371E−14 | 3.09321E−12 |
| Gabra4 | 70.48625962 | 9.560717973 | 1.380415948 | 6.925968936 | 4.33001E−12 | 7.7123E−10 |
| Meiob | 68.47190939 | 9.518853724 | 1.41227983 | 6.740062076 | 1.58319E−11 | 2.50654E−09 |
| Ccbe1 | 62.01845392 | 9.376771121 | 1.404547913 | 6.676006587 | 2.45541E−11 | 3.68721E−09 |
| Osmr | 119.1878792 | 9.355106288 | 1.313307689 | 7.123316468 | 1.05361E−12 | 2.14469E−10 |
| Rbpjl | 58.81657363 | 9.299888891 | 1.382530217 | 6.726716549 | 1.73534E−11 | 2.68771E−09 |
| Scgb3a1 | 46.69332853 | 8.966792973 | 1.526451009 | 5.874274981 | 4.24698E−09 | 3.83008E−07 |
| Tacr1 | 90.22608732 | 8.953093419 | 1.442760128 | 6.205531498 | 5.45124E−10 | 5.92936E−08 |
| Shisa7 | 44.47811575 | 8.896761389 | 1.466516304 | 6.066595622 | 1.3065E−09 | 1.32974E−07 |
| Klf15 | 43.62208825 | 8.868516729 | 1.689836489 | 5.248150803 | 1.53634E−07 | 9.55949E−06 |
| Cntn1 | 42.66670909 | 8.837519537 | 1.546864181 | 5.713183902 | 1.10882E−08 | 9.02831E−07 |
| Kcnb2 | 40.07510205 | 8.746624563 | 1.487788833 | 5.878942204 | 4.12896E−09 | 3.77139E−07 |
| Zkscan2 | 35.32711577 | 8.564166961 | 1.632782904 | 5.245135126 | 1.56168E−07 | 9.67494E−06 |
| Rassf10 | 28.25878766 | 8.242779172 | 1.804236973 | 4.56856793 | 4.91068E−06 | 0.00020107 |
| Antxr1 | 28.01025854 | 8.230769419 | 1.889902449 | 4.355129242 | 1.32988E−05 | 0.000480952 |
| C4b | 27.7848005 | 8.217839019 | 1.620800974 | 5.070233269 | 3.97328E−07 | 2.13643E−05 |
| Aplp1 | 27.07843828 | 8.180310689 | 1.676692656 | 4.87883731 | 1.06713E−06 | 5.31662E−05 |
| Nap1l2 | 23.73857492 | 7.990686805 | 1.66972541 | 4.785629277 | 1.70452E−06 | 7.86012E−05 |
| Panct2 | 23.35209493 | 7.967526953 | 1.735632944 | 4.590559876 | 4.42059E−06 | 0.000184336 |
| Zfp345 | 22.17833778 | 7.892454878 | 2.164280894 | 3.646686944 | 0.000265643 | 0.005970269 |
| Elovl2 | 19.95213298 | 7.739313481 | 1.846665283 | 4.19096712 | 2.77768E−05 | 0.000893435 |
| Gm20554 | 18.7309319 | 7.649583657 | 1.76612224 | 4.33128777 | 1.4824E−05 | 0.000530721 |
| Efemp1 | 17.20355042 | 7.526701843 | 2.109965546 | 3.56721552 | 0.000360795 | 0.007696054 |
| Nol3 | 16.56916686 | 7.471243946 | 1.888335997 | 3.956522546 | 7.60487E−05 | 0.002128916 |
| Spint1 | 171.7996551 | 7.421555267 | 0.815748624 | 9.097845889 | 9.21411E−20 | 4.23522E−17 |
| Cytl1 | 15.79960413 | 7.403110688 | 2.333458524 | 3.172591504 | 0.001510849 | 0.024161721 |
| Scara3 | 15.31217438 | 7.357453901 | 2.034912209 | 3.615612442 | 0.000299638 | 0.006640042 |
| Susd2 | 15.16120317 | 7.345046038 | 2.364004475 | 3.10703559 | 0.001889736 | 0.028433849 |
| Marveld3 | 14.22269444 | 7.250662887 | 2.007407325 | 3.611953985 | 0.000303898 | 0.006692813 |
| Popdc2 | 12.69919537 | 7.088579901 | 1.984593392 | 3.571804647 | 0.00035453 | 0.007585128 |
| Ptprz1 | 12.4505377 | 7.061062056 | 2.139796949 | 3.299874813 | 0.00096728 | 0.016973852 |
| 1700030C10Rik | 11.55865212 | 6.953624108 | 2.05872316 | 3.37763923 | 0.000731109 | 0.013564553 |
| Rcvrn | 42.4273902 | 6.947493739 | 1.382445803 | 5.025508939 | 5.02099E−07 | 2.64978E−05 |
| Rasgrf2 | 10.63015867 | 6.831450317 | 2.0760447 | 3.290608491 | 0.000999709 | 0.017499828 |
| Dpyd | 9.371599887 | 6.649621966 | 2.297963368 | 2.893702337 | 0.003807289 | 0.047629553 |
| Ripk4 | 48.83604089 | 6.572096075 | 1.412201123 | 4.653796099 | 3.25879E−06 | 0.000139863 |
| Nos1 | 8.831672842 | 6.563495614 | 2.243801856 | 2.925167209 | 0.003442712 | 0.044154096 |
| Pcdhb20 | 16.01287153 | 6.446575836 | 1.749200077 | 3.685442232 | 0.000228306 | 0.005281056 |
| Ces1g | 15.23467516 | 6.373675726 | 1.898429736 | 3.357340862 | 0.00078696 | 0.014427979 |
| Ccdc79 | 14.55849374 | 6.30703989 | 1.987862697 | 3.172774406 | 0.001509898 | 0.024161721 |
| Sbspon | 2209.626159 | 6.256992979 | 1.083817445 | 5.773105989 | 7.78235E−09 | 6.65839E−07 |
| Mt2 | 561.5331389 | 6.212775857 | 1.028586502 | 6.040110232 | 1.54009E−09 | 1.50306E−07 |
| Slc16a5 | 13.45274983 | 6.191490243 | 1.999505065 | 3.096511408 | 0.001958123 | 0.029154962 |
| Gabrb1 | 12.94385163 | 6.136273397 | 2.100802816 | 2.920918304 | 0.003490013 | 0.044480501 |
| Zg16 | 298.9344361 | 6.009510418 | 1.163308512 | 5.165878489 | 2.39312E−07 | 1.38616E−05 |
| Aass | 11.66444494 | 5.982302897 | 2.007524345 | 2.979940399 | 0.002883045 | 0.039012825 |
| Gprc5c | 42.57996683 | 5.951853739 | 1.175441485 | 5.063504917 | 4.11618E−07 | 2.19668E−05 |
| Elane | 10.92796593 | 5.887995831 | 2.006034515 | 2.935141837 | 0.003333953 | 0.043147584 |
| Gpr183 | 2118.133864 | 5.64790482 | 0.511302524 | 11.04611175 | 2.28916E−28 | 1.71675E−25 |
| Khdrbs2 | 14.95337051 | 5.411666354 | 1.709481032 | 3.165677918 | 0.00154722 | 0.024660334 |
| Gdf9 | 21.32829529 | 5.314276486 | 1.509685317 | 3.520121994 | 0.000431348 | 0.008894765 |
| Sntg2 | 68.58254983 | 5.015122829 | 0.957672771 | 5.236781271 | 1.63401E−07 | 9.99272E−06 |
| Dazl | 39.18817441 | 4.870517422 | 1.368130702 | 3.559979623 | 0.000370884 | 0.007875887 |
| Lox | 40.70503403 | 4.799642751 | 1.336986666 | 3.58989575 | 0.00033081 | 0.007185541 |
| Gipc2 | 137.1109726 | 4.745613271 | 0.768018148 | 6.179037935 | 6.44934E−10 | 6.90952E−08 |
| Cited4 | 101.7377166 | 4.732356198 | 0.824155103 | 5.742069885 | 9.35262E−09 | 7.67745E−07 |
| Trpc1 | 50.63749273 | 4.72775904 | 1.013486391 | 4.664847089 | 3.08847E−06 | 0.000132953 |
| Tram1l1 | 28.27692328 | 4.688871786 | 1.394715013 | 3.361885218 | 0.000774123 | 0.014251263 |
| Abi3bp | 66.11220416 | 4.649653482 | 1.181657261 | 3.934857962 | 8.32459E−05 | 0.002298781 |
| Pcdhgb2 | 16.61404557 | 4.615377064 | 1.593622129 | 2.89615523 | 0.003777655 | 0.047341956 |
| Agtr1a | 207.4272647 | 4.585023351 | 0.71678725 | 6.396630731 | 1.58843E−10 | 2.02085E−08 |
| Eya4 | 56.25897218 | 4.579187496 | 1.213025276 | 3.77501408 | 0.000159999 | 0.003910498 |
| Bmpr1a | 461.7613894 | 4.563698155 | 1.040603621 | 4.385625863 | 1.15653E−05 | 0.000422548 |
| Tmem215 | 130.6477024 | 4.388401721 | 0.761214331 | 5.765001446 | 8.16571E−09 | 6.88481E−07 |
| Chrm3 | 154.7646229 | 4.346969016 | 0.450311163 | 9.653256183 | 4.76192E−22 | 2.82719E−19 |
| Prom2 | 26.04563719 | 4.338472594 | 1.455187609 | 2.981383684 | 0.00286949 | 0.038866319 |
| Selp | 1964.535113 | 4.326139958 | 0.292661624 | 14.7820541 | 1.91244E−49 | 3.40629E−46 |
| Zswim5 | 79.4037549 | 4.21212101 | 0.936985614 | 4.495395604 | 6.94407E−06 | 0.000276386 |
| Ndst3 | 28.56177676 | 4.178353384 | 1.100605719 | 3.796412567 | 0.000146805 | 0.00367632 |
| Matn4 | 139.881751 | 4.149168181 | 0.788143065 | 5.26448606 | 1.40582E−07 | 8.86351E−06 |
| Tmod2 | 38.56117103 | 4.144377404 | 1.296829058 | 3.195777716 | 0.001394545 | 0.022683644 |
| Plscr2 | 2941.794427 | 3.980629266 | 0.238127736 | 16.71636127 | 9.96189E−63 | 7.09735E−59 |
| Pak7 | 49.40237256 | 3.966376505 | 1.250146632 | 3.172729025 | 0.001510134 | 0.024161721 |
| Gm6623 | 36.7734087 | 3.960463102 | 0.965541286 | 4.101806064 | 4.09938E−05 | 0.001253477 |
| Rprm | 63.22168367 | 3.847683953 | 0.906936676 | 4.24250563 | 2.21038E−05 | 0.000739336 |
| Wwtr1 | 334.3193101 | 3.831360447 | 1.228846664 | 3.117850712 | 0.001821751 | 0.027762699 |
| Muc1 | 119.5108139 | 3.719253074 | 0.536874997 | 6.927595981 | 4.28052E−12 | 7.7123E−10 |
| Zcchc14 | 91.52915615 | 3.634185369 | 0.952280478 | 3.81629725 | 0.000135469 | 0.0034286 |
| Clca3a1 | 2354.527995 | 3.585999029 | 0.53716125 | 6.675833428 | 2.45831E−11 | 3.68721E−09 |
| Tgfb3 | 39.98164187 | 3.539768859 | 1.188753298 | 2.977715278 | 0.002904056 | 0.039222651 |
| Gm5833 | 887.1450602 | 3.53623638 | 0.792522062 | 4.462003707 | 8.11968E−06 | 0.000315252 |
| H2-T3 | 117.155829 | 3.521013135 | 1.013663316 | 3.473552885 | 0.000513616 | 0.010293272 |
| Sept5 | 57.24441598 | 3.500302015 | 0.76252972 | 4.590381101 | 4.42437E−06 | 0.000184336 |
| Cldn5 | 934.1189281 | 3.490798354 | 0.314785311 | 11.08945757 | 1.41141E−28 | 1.11729E−25 |
| Rnf43 | 26.45057737 | 3.474169986 | 1.138333687 | 3.051978542 | 0.002273384 | 0.032490915 |
| Tc2n | 457.9572126 | 3.457237028 | 0.365879014 | 9.449126334 | 3.41671E−21 | 1.80313E−18 |
| Rgn | 92.9313366 | 3.447709595 | 1.143654008 | 3.014643913 | 0.002572811 | 0.035941164 |
| Nupr1 | 10482.14513 | 3.417296032 | 0.356258021 | 9.592193939 | 8.62315E−22 | 4.91485E−19 |
| Zmynd15 | 35.45487971 | 3.371030339 | 1.136264327 | 2.966765971 | 0.003009499 | 0.040094556 |
| Isl1 | 76.02738624 | 3.327229314 | 0.923278538 | 3.603711316 | 0.000313705 | 0.006890605 |
| Abca4 | 130.2816158 | 3.313847987 | 0.614397341 | 5.393656131 | 6.90383E−08 | 4.72945E−06 |
| Prkcz | 59.67562094 | 3.305327719 | 0.807143309 | 4.095093996 | 4.21997E−05 | 0.00127937 |
| Mapk11 | 37.40353211 | 3.302538628 | 1.036557212 | 3.186064975 | 0.001442222 | 0.023352525 |
| Abat | 42.45657187 | 3.287683371 | 0.900915734 | 3.649268459 | 0.000262988 | 0.005919934 |
| Cd200r4 | 166.5237405 | 3.279623928 | 0.541546352 | 6.056035493 | 1.39517E−09 | 1.3902E−07 |
| Rorb | 144.7247585 | 3.247226541 | 0.59302324 | 5.475715493 | 4.35747E−08 | 3.12008E−06 |
| Klrb1c | 1564.768926 | 3.204059021 | 0.210056903 | 15.25329076 | 1.56541E−52 | 5.57637E−49 |
| Map3k9 | 39.58896636 | 3.175428263 | 0.921039531 | 3.447656865 | 0.000565472 | 0.011159847 |
| Tm4sf1 | 1233.287685 | 3.156924232 | 0.258870386 | 12.19499951 | 3.30506E−34 | 3.36384E−31 |
| Dnm3 | 235.5541925 | 3.156140506 | 0.597574972 | 5.281580814 | 1.28074E−07 | 8.14699E−06 |
| Shc4 | 61.54022948 | 3.116417751 | 1.058412695 | 2.944425899 | 0.003235544 | 0.042143359 |
| Clu | 5958.622838 | 3.110391186 | 0.153257047 | 20.29525719 | 1.41612E−91 | 2.01783E−87 |
| Hoxb7 | 92.22943988 | 3.01677178 | 0.957969279 | 3.149132073 | 0.001637562 | 0.025868754 |
| Emp2 | 151.9479279 | 2.976461838 | 0.613086897 | 4.854877594 | 1.20461E−06 | 5.85819E−05 |
| Ramp2 | 1620.602415 | 2.973265913 | 0.264698945 | 11.23263228 | 2.81951E−29 | 2.36325E−26 |
| Aldh3a1 | 124.0845943 | 2.942449807 | 0.907948192 | 3.240768398 | 0.00119208 | 0.020084505 |
| Dnm3os | 64.71856405 | 2.924831938 | 0.94088965 | 3.108581264 | 0.001879879 | 0.028405516 |
| Gpx3 | 596.6232668 | 2.890003416 | 0.493166505 | 5.860096714 | 4.62598E−09 | 4.14563E−07 |
| Art2b | 60.86923766 | 2.859107703 | 0.983813727 | 2.906147398 | 0.00365909 | 0.046017987 |
| Rorc | 1154.269933 | 2.853768782 | 0.245403556 | 11.62888113 | 2.93925E−31 | 2.61758E−28 |
| 1700012l11Rik | 41.45014504 | 2.831650044 | 0.915756989 | 3.09214134 | 0.001987182 | 0.029395106 |
| Cd14 | 87.00634429 | 2.812782289 | 0.96460009 | 2.916008736 | 0.003545406 | 0.045025389 |
| Cystm1 | 69.23607369 | 2.786550757 | 0.708454648 | 3.933280363 | 8.37944E−05 | 0.002309452 |
| Npas3 | 65.20275156 | 2.77857915 | 0.898449908 | 3.09263669 | 0.001983868 | 0.029384763 |
| Ehd3 | 1493.489026 | 2.76375836 | 0.297883892 | 9.277971845 | 1.72735E−20 | 8.79037E−18 |
| Afap1 | 38.68189885 | 2.756166446 | 0.916295803 | 3.007943983 | 0.002630216 | 0.036563854 |
| Ncam1 | 81.93444718 | 2.721419596 | 0.778329726 | 3.496486778 | 0.000471428 | 0.009596251 |
| Gm8909 | 101.0675199 | 2.713569568 | 0.713466478 | 3.803359586 | 0.000142747 | 0.003593641 |
| Pgr | 332.3886423 | 2.705802706 | 0.541023665 | 5.001264978 | 5.69554E−07 | 2.98367E−05 |
| Fhdc1 | 289.1652518 | 2.698050004 | 0.563056542 | 4.7917923 | 1.65298E−06 | 7.64718E−05 |
| Mt1 | 3375.166007 | 2.603250747 | 0.20758411 | 12.54070336 | 4.4701E−36 | 5.30788E−33 |
| Il1rapl2 | 310.9879532 | 2.581644134 | 0.347351309 | 7.432371967 | 1.06667E−13 | 2.57611E−11 |
| Dlec1 | 40.39640738 | 2.567510456 | 0.858457717 | 2.990840907 | 0.002782104 | 0.037986228 |
| Gstm2 | 2762.498282 | 2.56681946 | 0.170182537 | 15.08274293 | 2.10346E−51 | 5.99443E−48 |
| Ar | 146.2542468 | 2.549570083 | 0.570186295 | 4.471468544 | 7.76843E−06 | 0.000303961 |
| Pclo | 159.1862323 | 2.543375416 | 0.836005611 | 3.042294672 | 0.002347819 | 0.033387303 |
| Bmp4 | 552.782805 | 2.541694175 | 0.3773308 | 6.735983853 | 1.62824E−11 | 2.54954E−09 |
| Nova1 | 59.52809219 | 2.520689259 | 0.876120873 | 2.877102163 | 0.004013456 | 0.049673439 |
| Acpp | 186.8576193 | 2.487800603 | 0.656006474 | 3.792341543 | 0.000149233 | 0.003711043 |
| Crip2 | 85.24461602 | 2.481079242 | 0.577147634 | 4.298864098 | 1.71676E−05 | 0.000595184 |
| Hsf4 | 35.7551283 | 2.478681085 | 0.801058787 | 3.094256157 | 0.001973071 | 0.029316248 |
| Tgfb1i1 | 136.8643701 | 2.463232305 | 0.597876185 | 4.119970601 | 3.78921E−05 | 0.001173748 |
| Ttc23 | 127.0645472 | 2.43098106 | 0.625942829 | 3.883711016 | 0.000102874 | 0.002719581 |
| Adgrg2 | 579.7308357 | 2.418636543 | 0.302984303 | 7.982712373 | 1.43152E−15 | 4.53284E−13 |
| Efemp2 | 151.1281194 | 2.415530893 | 0.429003176 | 5.630566463 | 1.79619E−08 | 1.38466E−06 |
| Tmem56 | 269.2849068 | 2.413136735 | 0.568871569 | 4.241971066 | 2.21565E−05 | 0.000739364 |
| Amotl2 | 276.2276402 | 2.412136967 | 0.391627274 | 6.159267062 | 7.30824E−10 | 7.71371E−08 |
| Ptprk | 260.0920791 | 2.408818664 | 0.501569137 | 4.802565559 | 1.56646E−06 | 7.36647E−05 |
| Meis2 | 89.53281427 | 2.405345524 | 0.615933499 | 3.905203282 | 9.41462E−05 | 0.00252719 |
| Skap1 | 195.1252771 | 2.393995376 | 0.375863743 | 6.369317124 | 1.89872E−10 | 2.31238E−08 |
| Sfrp1 | 296.4104022 | 2.38475632 | 0.497031133 | 4.798001901 | 1.60256E−06 | 7.43968E−05 |
| Pih1d2 | 94.26286577 | 2.334362477 | 0.539196922 | 4.329331977 | 1.49562E−05 | 0.000532778 |
| Arhgef28 | 493.4673973 | 2.324434125 | 0.342694091 | 6.782825235 | 1.17848E−11 | 1.92236E−09 |
| Sh3bgrl2 | 186.4268063 | 2.299884728 | 0.468029141 | 4.913977629 | 8.92469E−07 | 4.57439E−05 |
| Ffar2 | 97.21888651 | 2.29392639 | 0.670241853 | 3.422535283 | 0.0006204 | 0.011978436 |
| Gpx8 | 317.7041438 | 2.276047697 | 0.557169558 | 4.085018043 | 4.40734E−05 | 0.001327699 |
| Dsg2 | 253.5091034 | 2.269181924 | 0.329297373 | 6.890980941 | 5.5409E−12 | 9.74719E−10 |
| Gadd45g | 1141.003479 | 2.244524882 | 0.246655598 | 9.099833546 | 9.04704E−20 | 4.23522E−17 |
| Ackr1 | 156.4681092 | 2.242192193 | 0.458177084 | 4.893724004 | 9.89456E−07 | 4.96435E−05 |
| Fap | 243.492957 | 2.238776017 | 0.52989908 | 4.224910175 | 2.39036E−05 | 0.000792099 |
| Map1b | 502.239146 | 2.237588814 | 0.292200324 | 7.657721866 | 1.8926E−14 | 5.39354E−12 |
| Phf11d | 930.6250662 | 2.225903771 | 0.411227853 | 5.412823463 | 6.20386E−08 | 4.35462E−06 |
| Il17rc | 61.9580252 | 2.209830457 | 0.644765447 | 3.427340078 | 0.000609525 | 0.011800441 |
| Gm9199 | 703.4807629 | 2.176205307 | 0.284485236 | 7.649624779 | 2.01567E−14 | 5.63161E−12 |
| Neo1 | 409.4788928 | 2.175868193 | 0.449467782 | 4.840988123 | 1.29195E−06 | 6.19832E−05 |
| Hspb1 | 117.3333249 | 2.175441304 | 0.602039091 | 3.613455233 | 0.000302144 | 0.006664463 |
| Hoxb6 | 146.9088445 | 2.172419095 | 0.511591113 | 4.246397248 | 2.17235E−05 | 0.00073177 |
| Kif5a | 76.9148023 | 2.162441556 | 0.653551349 | 3.308755401 | 0.000937117 | 0.016543317 |
| Ehd2 | 96.87920061 | 2.155685364 | 0.622264247 | 3.464260361 | 0.000531692 | 0.010581109 |
| Sdpr | 4678.231742 | 2.145958511 | 0.135637697 | 15.82125435 | 2.22025E−56 | 1.05454E−52 |
| Cyp26b1 | 416.9828785 | 2.137081514 | 0.49320246 | 4.333071477 | 1.47043E−05 | 0.000527763 |
| Clec1a | 2327.664152 | 2.125985506 | 0.154969167 | 13.71876453 | 7.83898E−43 | 1.24108E−39 |
| Exoc314 | 117.574951 | 2.125161517 | 0.548833327 | 3.872143714 | 0.000107882 | 0.00282925 |
| Aspa | 871.923836 | 2.12428811 | 0.258618108 | 8.213996021 | 2.13947E−16 | 7.43543E−14 |
| Tmem231 | 104.7110459 | 2.122416524 | 0.625395267 | 3.393720159 | 0.000689501 | 0.012995637 |
| Lgmn | 98.59854649 | 2.099685428 | 0.671672717 | 3.126054365 | 0.001771688 | 0.027208451 |
| Nrg4 | 571.4584358 | 2.080683364 | 0.232172679 | 8.961792474 | 3.19443E−19 | 1.42242E−16 |
| 9530026P05Rik | 90.93901094 | 2.077276716 | 0.56407567 | 3.682620661 | 0.000230849 | 0.00532259 |
| Nxph1 | 51.44064617 | 2.074395414 | 0.698067609 | 2.971625365 | 0.002962279 | 0.039808186 |
| Zfp92 | 104.8548233 | 2.048156544 | 0.659800989 | 3.104203507 | 0.00190792 | 0.028616799 |
| C130026l21Rik | 81.50112212 | 2.044142622 | 0.680617975 | 3.003362673 | 0.00267014 | 0.036781666 |
| Slc16a12 | 320.2163372 | 2.038738555 | 0.447833339 | 4.552449266 | 5.3025E−06 | 0.000216491 |
| Zfp57 | 266.4061294 | 2.014185439 | 0.364014881 | 5.533250278 | 3.1435E−08 | 2.32082E−06 |
| Jam2 | 1571.492923 | 2.006805471 | 0.216483473 | 9.270016966 | 1.86116E−20 | 9.14473E−18 |
| Maf | 200.4607638 | 1.999750584 | 0.394866401 | 5.064372606 | 4.09748E−07 | 2.19492E−05 |
| Nr1h3 | 148.1243537 | 1.994293763 | 0.462252065 | 4.314299309 | 1.6011E−05 | 0.000559168 |
| Gpr4 | 203.7004868 | 1.971689736 | 0.58732708 | 3.357055725 | 0.000787772 | 0.014427979 |
| Rab34 | 263.7825431 | 1.966396711 | 0.507870953 | 3.871843226 | 0.000108015 | 0.00282925 |
| Irf2bpl | 149.5842055 | 1.955404745 | 0.45448723 | 4.302441556 | 1.68926E−05 | 0.00058708 |
| Enpp5 | 5616.301704 | 1.939852694 | 0.162939676 | 11.90534276 | 1.11006E−32 | 1.05449E−29 |
| Cd38 | 1824.085275 | 1.929889453 | 0.155046573 | 12.44715968 | 1.44911E−35 | 1.58834E−32 |
| Cxcl16 | 181.6418571 | 1.926281258 | 0.609534194 | 3.160251346 | 0.001576331 | 0.025096245 |
| Serpinb8 | 443.5450853 | 1.918276179 | 0.40402831 | 4.747875663 | 2.05564E−06 | 9.3283E−05 |
| Ptafr | 87.28442725 | 1.915605822 | 0.607618722 | 3.152644502 | 0.001617987 | 0.025587902 |
| Meis3 | 315.6727297 | 1.901135964 | 0.324478942 | 5.859042657 | 4.65543E−09 | 4.14595E−07 |
| Sult1a1 | 14975.4053 | 1.892094378 | 0.181488807 | 10.42540532 | 1.89848E−25 | 1.28816E−22 |
| Kdr | 192.289582 | 1.889692799 | 0.547628074 | 3.450686497 | 0.000559163 | 0.011065985 |
| Dhrs3 | 343.3786633 | 1.887622502 | 0.428468189 | 4.405513759 | 1.05533E−05 | 0.000396767 |
| Rbm19 | 499.1169208 | 1.877842088 | 0.325614416 | 5.767072947 | 8.06601E−09 | 6.84123E−07 |
| Dpy19l2 | 160.2871646 | 1.865449377 | 0.473255646 | 3.941737185 | 8.08936E−05 | 0.002246886 |
| Fam46c | 177.4112848 | 1.862725999 | 0.466882124 | 3.989713688 | 6.61531E−05 | 0.001873987 |
| Entpd2 | 153.1795921 | 1.860906802 | 0.594135378 | 3.132125897 | 0.001735454 | 0.026849609 |
| Lpl | 452.490044 | 1.842225613 | 0.28296752 | 6.510378342 | 7.49618E−11 | 1.02705E−08 |
| Gm13546 | 270.0168318 | 1.837773969 | 0.347070326 | 5.295105433 | 1.18948E−07 | 7.63462E−06 |
| Ampd3 | 451.025726 | 1.836133209 | 0.357850885 | 5.131000882 | 2.88206E−07 | 1.61679E−05 |
| Gm9958 | 226.4433984 | 1.832890778 | 0.460930378 | 3.976502452 | 6.99363E−05 | 0.001969411 |
| Smtnl2 | 67.11615099 | 1.831463949 | 0.593218804 | 3.08733293 | 0.002019613 | 0.029728781 |
| Hpgds | 786.7310286 | 1.819413498 | 0.371767316 | 4.893957642 | 9.88281E−07 | 4.96435E−05 |
| Dlk1 | 325.72222 | 1.797512381 | 0.402496432 | 4.465908854 | 7.97296E−06 | 0.000310401 |
| Rundc3a | 121.4751211 | 1.79547212 | 0.559509987 | 3.209008171 | 0.001331937 | 0.021839783 |
| Gulo | 99.14996111 | 1.780789772 | 0.60027342 | 2.96663106 | 0.00301082 | 0.040094556 |
| Cysltr2 | 1004.187476 | 1.767771212 | 0.235020855 | 7.52176316 | 5.40424E−14 | 1.45293E−11 |
| Ppap2a | 132.9912743 | 1.728865996 | 0.469831457 | 3.679757862 | 0.000233456 | 0.005365335 |
| C1qtnf4 | 141.7277682 | 1.711035007 | 0.466096542 | 3.670988419 | 0.000241614 | 0.00549962 |
| Pde9a | 592.8933351 | 1.709798627 | 0.215722342 | 7.925922809 | 2.26458E−15 | 7.01479E−13 |
| Dcbld2 | 152.5912765 | 1.689096992 | 0.450305729 | 3.751000449 | 0.00017613 | 0.004246501 |
| Alcam | 2565.137063 | 1.682831782 | 0.127452491 | 13.20360057 | 8.36397E−40 | 1.19178E−36 |
| Prtn3 | 8497.608822 | 1.676364457 | 0.11291761 | 14.84590812 | 7.39466E−50 | 1.50524E−46 |
| Mmp14 | 297.4552488 | 1.671850886 | 0.492247907 | 3.39635956 | 0.000682886 | 0.012905095 |
| Rab40b | 242.1181753 | 1.633480099 | 0.342708281 | 4.766386423 | 1.87559E−06 | 8.56581E−05 |
| Klhl13 | 210.1446988 | 1.627323978 | 0.481004065 | 3.38318134 | 0.000716513 | 0.013373814 |
| Ctsw | 921.5067028 | 1.622828922 | 0.259965165 | 6.242486068 | 4.3067E−10 | 4.72048E−08 |
| Rdh10 | 685.6202947 | 1.61791673 | 0.316490444 | 5.112055548 | 3.18672E−07 | 1.77373E−05 |
| Cpne8 | 2008.895172 | 1.613091178 | 0.153015927 | 10.54198217 | 5.53204E−26 | 3.9413E−23 |
| Epdr1 | 741.3928489 | 1.602278689 | 0.284612744 | 5.629680062 | 1.80544E−08 | 1.38466E−06 |
| Echdc2 | 144.3716492 | 1.602206355 | 0.496132669 | 3.229390955 | 0.001240542 | 0.020698454 |
| 1810041H14Rik | 83.50564396 | 1.597553574 | 0.554926181 | 2.878857815 | 0.003991182 | 0.049538638 |
| Ccdc92 | 242.4559404 | 1.593326736 | 0.413448175 | 3.853752014 | 0.000116321 | 0.003002652 |
| Plek | 1719.975563 | 1.593176124 | 0.245433314 | 6.491279034 | 8.51107E−11 | 1.12291E−08 |
| Tmem150a | 484.7680207 | 1.592738117 | 0.263581641 | 6.042674715 | 1.5158E−09 | 1.48956E−07 |
| Lsr | 370.6447433 | 1.590264075 | 0.284668812 | 5.586365663 | 2.31871E−08 | 1.75741E−06 |
| Gda | 2573.490874 | 1.589070898 | 0.238627925 | 6.659199247 | 2.75323E−11 | 4.08655E−09 |
| Nxnl2 | 300.9684225 | 1.57959984 | 0.306722112 | 5.149937936 | 2.60573E−07 | 1.49112E−05 |
| Pigg | 329.0269646 | 1.565775417 | 0.356463855 | 4.392522259 | 1.12043E−05 | 0.000413602 |
| Perp | 313.8383856 | 1.546314656 | 0.386050084 | 4.005476806 | 6.18925E−05 | 0.001774459 |
| 9230112J17Rik | 271.9845636 | 1.540221103 | 0.343806566 | 4.479906009 | 7.46759E−06 | 0.000294864 |
| Smtnl1 | 862.3831481 | 1.538780504 | 0.224867388 | 6.843057675 | 7.75204E−12 | 1.31499E−09 |
| Cdkn2c | 601.7928118 | 1.535894722 | 0.238884534 | 6.429443954 | 1.28072E−10 | 1.65899E−08 |
| Nuak2 | 336.8059202 | 1.53514246 | 0.426587919 | 3.598654329 | 0.000319868 | 0.007010085 |
| Gkn3 | 384.9752643 | 1.534541103 | 0.498981712 | 3.075345384 | 0.002102589 | 0.030477917 |
| Aph1b | 293.2878667 | 1.526396432 | 0.407900903 | 3.742076622 | 0.000182506 | 0.00437799 |
| Epcam | 403.134906 | 1.526352719 | 0.332957924 | 4.584221038 | 4.55682E−06 | 0.000188203 |
| Arpin | 470.4475776 | 1.520691029 | 0.336061461 | 4.525038435 | 6.03845E−06 | 0.000244437 |
| Apol7e | 540.2597369 | 1.518525566 | 0.359217254 | 4.227318011 | 2.36493E−05 | 0.0007855 |
| Fam57a | 152.9401628 | 1.517663506 | 0.480567579 | 3.1580647 | 0.001588203 | 0.02522888 |
| Npdc1 | 2158.161937 | 1.515565166 | 0.187959616 | 8.063248888 | 7.4293E−16 | 2.40591E−13 |
| Ldhd | 353.1406355 | 1.508397126 | 0.271246628 | 5.560980195 | 2.68264E−08 | 2.0013E−06 |
| Rarb | 1015.782841 | 1.491397772 | 0.280208798 | 5.322451627 | 1.02378E−07 | 6.69167E−06 |
| Klhl4 | 2296.958611 | 1.48344917 | 0.199243495 | 7.4454083 | 9.6645E−14 | 2.3743E−11 |
| Calml4 | 465.4183473 | 1.47125947 | 0.332802943 | 4.420812673 | 9.83304E−06 | 0.000373629 |
| Shtn1 | 239.2543603 | 1.450565258 | 0.3689231 | 3.931890571 | 8.42804E−05 | 0.002318363 |
| 4921504A21Rik | 194.9198781 | 1.437658457 | 0.439543553 | 3.270798645 | 0.001072442 | 0.018500281 |
| Vldlr | 775.9600936 | 1.436547985 | 0.227399587 | 6.317284929 | 2.66199E−10 | 3.10907E−08 |
| Dcun1d4 | 305.9038175 | 1.429603823 | 0.447490247 | 3.194715043 | 0.00139969 | 0.022729077 |
| Runx1t1 | 750.4681443 | 1.429343375 | 0.234290728 | 6.100725322 | 1.05588E−09 | 1.09024E−07 |
| Srd5a1 | 325.1095036 | 1.414682256 | 0.3669729 | 3.85500471 | 0.000115727 | 0.002992909 |
| A230065H16Rik | 291.3074819 | 1.4076396 | 0.421182954 | 3.342109615 | 0.000831442 | 0.015115768 |
| Plbd1 | 212.2854781 | 1.402244197 | 0.429028601 | 3.268416592 | 0.00108151 | 0.018624839 |
| Ocln | 446.3636477 | 1.394275465 | 0.483351377 | 2.88460017 | 0.003919111 | 0.048899655 |
| Rasl11a | 198.4245836 | 1.393630548 | 0.371622446 | 3.75012479 | 0.000176747 | 0.004254159 |
| Pros1 | 1101.986687 | 1.369272907 | 0.327427722 | 4.181908904 | 2.89072E−05 | 0.000921473 |
| Oxr1 | 12230.70433 | 1.366469367 | 0.10697174 | 12.77411557 | 2.28733E−37 | 2.96292E−34 |
| Fhl1 | 3118.847314 | 1.365408939 | 0.168488667 | 8.103862186 | 5.32414E−16 | 1.80628E−13 |
| Pam | 823.2316177 | 1.365227251 | 0.182690955 | 7.472878188 | 7.84595E−14 | 1.96135E−11 |
| Plcl1 | 696.8679763 | 1.354376181 | 0.21179806 | 6.394658093 | 1.60907E−10 | 2.02899E−08 |
| Fam169a | 250.0658072 | 1.349885729 | 0.34301512 | 3.935353432 | 8.30744E−05 | 0.002298498 |
| Aldh1a1 | 4772.323795 | 1.346027241 | 0.129465511 | 10.39680166 | 2.56395E−25 | 1.66062E−22 |
| 1700029J07Rik | 684.2496322 | 1.340968525 | 0.302226756 | 4.436961655 | 9.12375E−06 | 0.000349474 |
| 2410017l17Rik | 180.5696759 | 1.33727344 | 0.44926953 | 2.976550491 | 0.002915111 | 0.039334672 |
| Glipr1l1 | 423.5368688 | 1.330467302 | 0.307654917 | 4.324544241 | 1.52848E−05 | 0.000541138 |
| Heg1 | 931.8311036 | 1.303929106 | 0.202395416 | 6.4424834 | 1.17534E−10 | 1.53646E−08 |
| Vwf | 1720.439387 | 1.298342607 | 0.228375025 | 5.685133957 | 1.3071E−08 | 1.03472E−06 |
| Eps8l2 | 218.6312427 | 1.295629127 | 0.415288813 | 3.1198267 | 0.001809575 | 0.027636259 |
| Gstm6 | 152.70394 | 1.295561757 | 0.450216056 | 2.877644502 | 0.004006564 | 0.049643066 |
| Neu3 | 299.5930327 | 1.286187276 | 0.38636244 | 3.328965606 | 0.000871692 | 0.015643243 |
| Rbpms2 | 939.5123515 | 1.279368283 | 0.217694061 | 5.876909452 | 4.17997E−09 | 3.79365E−07 |
| Gem | 1700.15707 | 1.269384618 | 0.259741002 | 4.887116816 | 1.02323E−06 | 5.11581E−05 |
| Itgb3 | 1775.814631 | 1.268121702 | 0.211566444 | 5.993964237 | 2.04786E−09 | 1.93245E−07 |
| Cebpd | 258.1223354 | 1.257124657 | 0.303152605 | 4.146837715 | 3.37099E−05 | 0.001058 |
| Bhlhe40 | 433.7098779 | 1.247904173 | 0.405358055 | 3.078523189 | 0.002080293 | 0.030308896 |
| Rnf208 | 285.083629 | 1.244623246 | 0.407370975 | 3.05525755 | 0.002248673 | 0.032233329 |
| Ppargc1a | 163.4026831 | 1.242671624 | 0.382800482 | 3.246264522 | 0.001169301 | 0.019811385 |
| Bend7 | 154.7152903 | 1.240717929 | 0.431701404 | 2.874018751 | 0.004052849 | 0.049955922 |
| Zfp354a | 428.3012542 | 1.238751267 | 0.3990114 | 3.104551063 | 0.00190568 | 0.028613318 |
| Mill2 | 187.893856 | 1.238294309 | 0.430017127 | 2.879639509 | 0.003981301 | 0.049459075 |
| Bcl3 | 984.0554197 | 1.234338764 | 0.240398906 | 5.134544013 | 2.82829E−07 | 1.59296E−05 |
| Rapgef3 | 195.3106186 | 1.228752676 | 0.396317625 | 3.100424001 | 0.001932438 | 0.028863007 |
| Ptges | 213.4727335 | 1.221010236 | 0.412189499 | 2.962254591 | 0.003053952 | 0.040555224 |
| Col16a1 | 449.8342887 | 1.220281809 | 0.263450866 | 4.63191421 | 3.623E−06 | 0.000154563 |
| Fads3 | 512.5332386 | 1.212555752 | 0.309409317 | 3.918937429 | 8.89402E−05 | 0.002413922 |
| Ptger4 | 1149.167556 | 1.208006255 | 0.226240541 | 5.339477397 | 9.32149E−08 | 6.14916E−06 |
| Sox18 | 592.6998902 | 1.200510365 | 0.231828808 | 5.178434781 | 2.23755E−07 | 1.30147E−05 |
| Ypel2 | 227.7385545 | 1.19858235 | 0.326459298 | 3.67146029 | 0.000241169 | 0.005498257 |
| Ak1 | 658.2133615 | 1.193426441 | 0.258726809 | 4.61268952 | 3.97492E−06 | 0.000168067 |
| Nhsl2 | 154.9016906 | 1.192081003 | 0.40956721 | 2.910587013 | 0.003607505 | 0.045570336 |
| Pygm | 1014.21432 | 1.191575824 | 0.167260112 | 7.124088409 | 1.04772E−12 | 2.14469E−10 |
| Adamtsl5 | 380.6140161 | 1.183396188 | 0.287583429 | 4.114966544 | 3.87236E−05 | 0.001191732 |
| Slamf1 | 3241.247711 | 1.17155997 | 0.196290389 | 5.968503989 | 2.39439E−09 | 2.24458E−07 |
| B3gnt8 | 404.0044084 | 1.165901237 | 0.286618851 | 4.067775833 | 4.7464E−05 | 0.001403142 |
| Cdc14b | 306.8327056 | 1.163047885 | 0.377967098 | 3.077114097 | 0.002090153 | 0.030390395 |
| Efna1 | 1987.294291 | 1.161041484 | 0.204377878 | 5.680856938 | 1.34022E−08 | 1.05507E−06 |
| Id2 | 1834.410622 | 1.147897284 | 0.179869135 | 6.38184691 | 1.74965E−10 | 2.18691E−08 |
| B9d1 | 253.4558138 | 1.134566175 | 0.368690627 | 3.077285105 | 0.002088954 | 0.030390395 |
| Prune2 | 316.687701 | 1.134234725 | 0.349999491 | 3.240675356 | 0.001192469 | 0.020084505 |
| Bbs10 | 560.0282357 | 1.133861105 | 0.38985272 | 2.908434511 | 0.003632432 | 0.045804009 |
| H2-Eb1 | 2168.54257 | 1.131055617 | 0.25700746 | 4.400866873 | 1.07819E−05 | 0.000402177 |
| Mir155hg | 1219.819008 | 1.130184575 | 0.238792349 | 4.732917876 | 2.21315E−06 | 9.94801E−05 |
| Tmem50b | 1670.763049 | 1.124956032 | 0.209326837 | 5.37416057 | 7.69403E−08 | 5.19584E−06 |
| Art4 | 4357.182182 | 1.121766549 | 0.150066643 | 7.47512258 | 7.71319E−14 | 1.96135E−11 |
| Dock9 | 516.3348651 | 1.121372376 | 0.29928151 | 3.746881576 | 0.000179047 | 0.00430225 |
| Stom | 476.3033005 | 1.120251232 | 0.254918701 | 4.394543154 | 1.11006E−05 | 0.000410837 |
| Fyb | 3075.950362 | 1.116141869 | 0.214755296 | 5.197272854 | 2.02233E−07 | 1.20068E−05 |
| Srxn1 | 967.176831 | 1.108377653 | 0.289122097 | 3.833597173 | 0.000126283 | 0.00322474 |
| Pabpc4l | 454.3778874 | 1.1011729 | 0.286795255 | 3.839578512 | 0.000123246 | 0.003164195 |
| Evc2 | 258.6596656 | 1.098615627 | 0.321767456 | 3.414315547 | 0.000639425 | 0.012262669 |
| Ppp1r16b | 485.5334446 | 1.095197877 | 0.35969598 | 3.044787649 | 0.002328446 | 0.033144889 |
| Smarcd3 | 270.1158723 | 1.094892409 | 0.31039222 | 3.527447975 | 0.000419586 | 0.008689947 |
| St3gal6 | 849.1381399 | 1.092900671 | 0.241896402 | 4.51805261 | 6.2411E−06 | 0.000251495 |
| Tfcp2 | 303.0388621 | 1.085956953 | 0.3669024 | 2.959797898 | 0.003078409 | 0.040728182 |
| Manea | 700.7700352 | 1.08498414 | 0.209520419 | 5.178417197 | 2.23776E−07 | 1.30147E−05 |
| Camkk1 | 439.7126719 | 1.082982207 | 0.323694005 | 3.345697451 | 0.000820759 | 0.014955236 |
| Chac2 | 660.8567318 | 1.080782826 | 0.273726652 | 3.94840188 | 7.86747E−05 | 0.002193806 |
| Rbm38 | 1135.651954 | 1.074265062 | 0.28277047 | 3.799070894 | 0.00014524 | 0.003649944 |
| Selm | 2210.477056 | 1.069897119 | 0.159173071 | 6.721596246 | 1.79744E−11 | 2.75396E−09 |
| Xdh | 943.9429888 | 1.064226035 | 0.221759809 | 4.799003213 | 1.59457E−06 | 7.43968E−05 |
| Pbx3 | 908.7191151 | 1.062373768 | 0.203515216 | 5.220119591 | 1.78808E−07 | 1.07503E−05 |
| Tox | 1394.402636 | 1.054817836 | 0.174015253 | 6.06164009 | 1.3474E−09 | 1.36164E−07 |
| Rhoj | 764.9614706 | 1.054613517 | 0.203136546 | 5.191648368 | 2.0844E−07 | 1.2273E−05 |
| Fads2 | 393.99267 | 1.041974034 | 0.31886007 | 3.267809715 | 0.001083832 | 0.018629099 |
| Stk39 | 198.2003864 | 1.026518728 | 0.35492905 | 2.892180078 | 0.003825786 | 0.047818969 |
| Cd302 | 1221.920239 | 1.016368306 | 0.208442289 | 4.876017772 | 1.08249E−06 | 5.37435E−05 |
| Procr | 5651.121685 | 1.014760858 | 0.125459832 | 8.088332651 | 6.0487E−16 | 2.00437E−13 |
| Adck3 | 3632.644948 | 1.011827675 | 0.216821905 | 4.666630319 | 3.06179E−06 | 0.000132205 |
| Tacstd2 | 1930.656676 | 1.011676823 | 0.230621905 | 4.386733433 | 1.15066E−05 | 0.000422548 |
| Gstt1 | 1716.775936 | 1.010172966 | 0.183367294 | 5.50901388 | 3.60849E−08 | 2.62334E−06 |
| Lpar6 | 9913.552595 | 1.008966447 | 0.155100467 | 6.505244429 | 7.75672E−11 | 1.05262E−08 |
| Yipf2 | 461.3834107 | 1.008625485 | 0.346752299 | 2.908778078 | 0.003628443 | 0.045794232 |
| Rab3il1 | 665.5925094 | 1.002090491 | 0.228469113 | 4.386109248 | 1.15396E−05 | 0.000422548 |
| Plscr1 | 3942.018783 | 1.001494809 | 0.12018258 | 8.333111218 | 7.87451E−17 | 2.87702E−14 |
| Gchfr | 830.0150468 | 1.001463574 | 0.233613674 | 4.286836293 | 1.81236E−05 | 0.000622467 |
| Smagp | 813.8781206 | 1.001178075 | 0.214249352 | 4.672957318 | 2.96893E−06 | 0.000128585 |

TABLE 1B
List of DEGs between young and old HSCs derived from bulk RNA-seq
Down-regulated genes in old HSCs when compared with young HSCs

| Genes | baseMean | log2FoldChange | IfcSE | stat | pvalue | padj |
|---|---|---|---|---|---|---|
| Spns3 | 285.376115 | −1.003060007 | 0.327983065 | −3.058267675 | 0.002226206 | 0.032009296 |
| Gins2 | 886.039298 | −1.004339064 | 0.229830447 | −4.369913027 | 1.24296E−05 | 0.00045181 |
| Rev3l | 850.140504 | −1.004615372 | 0.240423179 | −4.178529607 | 2.934E−05 | 0.000933182 |
| Nt5dc2 | 1283.12923 | −1.005012181 | 0.192392011 | −5.223772946 | 1.75314E−07 | 1.063E−05 |
| Clspn | 444.852792 | −1.007286438 | 0.322356582 | −3.124758403 | 0.001779511 | 0.027264795 |
| Trf | 917.669287 | −1.013601738 | 0.251912428 | −4.023627357 | 5.73086E−05 | 0.00166651 |
| Myo7a | 271.53045 | −1.015375373 | 0.317632863 | −3.196694962 | 0.001390118 | 0.022637484 |
| Nmral1 | 1527.75713 | −1.017878117 | 0.194496232 | −5.233407912 | 1.66413E−07 | 1.01334E−05 |
| Afp | 648.742424 | −1.019797917 | 0.232853923 | −4.379560825 | 1.18919E−05 | 0.000433369 |
| Dusp6 | 1769.37116 | −1.022914824 | 0.28276837 | −3.617500874 | 0.000297461 | 0.006602066 |
| Syce2 | 2702.43517 | −1.023777629 | 0.141017133 | −7.259952073 | 3.87228E−13 | 8.7581E−11 |
| Acy1 | 532.652951 | −1.024543149 | 0.307800944 | −3.32859002 | 0.000872868 | 0.015644648 |
| Gpr65 | 766.604372 | −1.027778582 | 0.262002848 | −3.922776382 | 8.75344E−05 | 0.002387228 |
| Prim2 | 965.80022 | −1.030007444 | 0.256882557 | −4.009643377 | 6.08105E−05 | 0.001746954 |
| Atad5 | 830.091822 | −1.033320381 | 0.319643394 | −3.232728723 | 0.001226139 | 0.020506174 |
| Rad54l | 421.894574 | −1.034349483 | 0.247818052 | −4.173826229 | 2.99526E−05 | 0.000950546 |
| Dnmt1 | 1736.75296 | −1.034449438 | 0.196999115 | −5.251035952 | 1.51246E−07 | 9.45222E−06 |
| Gng2 | 1139.67928 | −1.044454122 | 0.226935667 | −4.602423824 | 4.17602E−06 | 0.000176048 |
| 4930579G24Rik | 949.563667 | −1.051675886 | 0.219192356 | −4.797958775 | 1.60291E−06 | 7.43968E−05 |
| Arhgap30 | 798.207636 | −1.061868841 | 0.229792147 | −4.620997091 | 3.819E−06 | 0.000162439 |
| Carns1 | 256.59195 | −1.064733427 | 0.339337192 | −3.137685619 | 0.001702874 | 0.026569378 |
| Rgcc | 800.623557 | −1.069651366 | 0.233829443 | −4.574493923 | 4.77372E−06 | 0.000196025 |
| Cd68 | 312.191056 | −1.072267245 | 0.348625846 | −3.075696356 | 0.002100116 | 0.030477917 |
| Kif20b | 1833.94757 | −1.073056493 | 0.169218413 | −6.341251375 | 2.27906E−10 | 2.7062E−08 |
| E030024N20Rik | 238.62727 | −1.076386046 | 0.3696724 | −2.911729539 | 0.003594337 | 0.045448669 |
| Dtl | 1068.73982 | −1.089348559 | 0.231795431 | −4.699611878 | 2.60656E−06 | 0.000115279 |
| Slfn9 | 842.518094 | −1.094731781 | 0.295045753 | −3.710379723 | 0.000206949 | 0.004874067 |
| Cd37 | 1030.0848 | −1.095180695 | 0.23219188 | −4.716705406 | 2.39694E−06 | 0.000106731 |
| Lsp1 | 5385.75339 | −1.095788819 | 0.126492053 | −8.662906418 | 4.59886E−18 | 1.82025E−15 |
| Rwdd2a | 408.218471 | −1.097584574 | 0.279259564 | −3.930338346 | 8.48264E−05 | 0.002319945 |
| Gins1 | 636.567042 | −1.100133363 | 0.315163523 | −3.490674789 | 0.000481802 | 0.009751707 |
| Szt2 | 319.937154 | −1.102674737 | 0.349753905 | −3.152716009 | 0.001617591 | 0.025587902 |
| C330027C09Rik | 448.979523 | −1.104419283 | 0.372561073 | −2.964397955 | 0.003032758 | 0.040311353 |
| Gab2 | 286.424144 | −1.115299043 | 0.327270349 | −3.407882948 | 0.00065469 | 0.012471489 |
| Tnik | 372.947261 | −1.117158779 | 0.376308145 | −2.968733985 | 0.002990293 | 0.040008161 |
| Ptgr1 | 457.948188 | −1.127724023 | 0.313936062 | −3.592209241 | 0.000327886 | 0.007149186 |
| Kif23 | 1231.64942 | −1.130434016 | 0.239206579 | −4.725764733 | 2.29251E−06 | 0.000102401 |
| Mad2l1 | 2086.50659 | −1.143496374 | 0.190024596 | −6.017622967 | 1.76997E−09 | 1.69264E−07 |
| Cdt1 | 799.788125 | −1.144301419 | 0.295212262 | −3.876198813 | 0.000106101 | 0.002794516 |
| Susd1 | 546.059382 | −1.144611345 | 0.291158394 | −3.931232516 | 8.45115E−05 | 0.002319945 |
| P2ry14 | 567.348513 | −1.149216418 | 0.287814591 | −3.992905345 | 6.52686E−05 | 0.001853055 |
| Fgl2 | 451.343696 | −1.15892242 | 0.287211604 | −4.035082158 | 5.45832E−05 | 0.001597035 |
| Ap1s2 | 273.093525 | −1.162770524 | 0.338976585 | −3.430238478 | 0.000603051 | 0.011690984 |
| Il15 | 2219.02575 | −1.164277196 | 0.163296845 | −7.129820504 | 1.005E−12 | 2.10592E−10 |
| Asf1b | 1519.9997 | −1.167131568 | 0.30381606 | −3.841572988 | 0.000122248 | 0.003144255 |
| Brip1 | 368.256003 | −1.17875801 | 0.38001861 | −3.101842856 | 0.0019232 | 0.028797512 |
| Rad51 | 1007.98302 | −1.188223981 | 0.331273662 | −3.586835041 | 0.000334716 | 0.007259311 |
| Wdhd1 | 681.28342 | −1.188461996 | 0.243956009 | −4.871624192 | 1.10685E−06 | 5.4762E−05 |
| Fignl1 | 1311.99704 | −1.189596647 | 0.208776239 | −5.697950355 | 1.21256E−08 | 9.76148E−07 |
| Nrm | 1947.54229 | −1.192721423 | 0.135027166 | −8.83319603 | 1.01727E−18 | 4.26324E−16 |
| Tacc3 | 929.450817 | −1.192910131 | 0.187358389 | −6.366996098 | 1.92766E−10 | 2.32773E−08 |
| Exo1 | 601.446575 | −1.200776122 | 0.377091757 | −3.18430753 | 0.001451008 | 0.023459077 |
| Serpinb1a | 1965.1794 | −1.204949315 | 0.169897783 | −7.09220152 | 1.31995E−12 | 2.57644E−10 |
| Col4a1 | 419.839151 | −1.210909475 | 0.399951611 | −3.027639951 | 0.002464716 | 0.034806474 |
| Depdc1b | 275.583411 | −1.216453428 | 0.387652565 | −3.137999174 | 0.001701054 | 0.026569378 |
| Cenpf | 902.669919 | −1.221755708 | 0.398186596 | −3.068299433 | 0.002152808 | 0.031079391 |
| Tyms | 3030.83598 | −1.221969906 | 0.195235193 | −6.258963277 | 3.87545E−10 | 4.38264E−08 |
| Cdc6 | 743.564791 | −1.234007593 | 0.256644655 | −4.808234152 | 1.52269E−06 | 7.18439E−05 |
| Slc28a2 | 348.525789 | −1.235707346 | 0.348012363 | −3.550757035 | 0.000384125 | 0.008108733 |
| Hells | 1581.85658 | −1.236963746 | 0.194476718 | −6.360472133 | 2.01135E−10 | 2.40837E−08 |
| Cd34 | 4235.32531 | −1.238016899 | 0.130153537 | −9.511972769 | 1.8708E−21 | 1.02527E−18 |
| Slc22a3 | 341.537131 | −1.238047045 | 0.275216501 | −4.498447732 | 6.84514E−06 | 0.000273211 |
| Tipin | 4767.11823 | −1.246316242 | 0.163331231 | −7.630605829 | 2.33653E−14 | 6.40254E−12 |
| Kctd12b | 220.66266 | −1.254301631 | 0.407852011 | −3.075384199 | 0.002102315 | 0.030477917 |
| Prim1 | 2874.51515 | −1.265776822 | 0.177914893 | −7.114507412 | 1.12313E−12 | 2.22271E−10 |
| Gmnn | 1965.17939 | −1.269462602 | 0.213375002 | −5.949443899 | 2.69055E−09 | 2.48946E−07 |
| Arxes2 | 546.482163 | −1.269551866 | 0.261588855 | −4.853233765 | 1.21464E−06 | 5.88689E−05 |
| Arsb | 287.822852 | −1.276164204 | 0.377764902 | −3.378196851 | 0.000729628 | 0.013554724 |
| Pole2 | 686.244066 | −1.278945761 | 0.312151985 | −4.097189264 | 4.18197E−05 | 0.001272158 |
| Pidd1 | 161.305876 | −1.280497586 | 0.420122425 | −3.047915343 | 0.002304348 | 0.03283466 |
| Hist1h1e | 327.880975 | −1.281842348 | 0.384024981 | −3.337913969 | 0.000844099 | 0.015263406 |
| Il4 | 739.947261 | −1.295391167 | 0.242067083 | −5.351372644 | 8.72896E−08 | 5.8121E−06 |
| Cryl1 | 496.731527 | −1.300197084 | 0.293144983 | −4.435337998 | 9.19279E−06 | 0.000351175 |
| Spry1 | 725.845669 | −1.303116867 | 0.40925872 | −3.184090655 | 0.001452095 | 0.023459077 |
| Nkg7 | 1345.25536 | −1.306051197 | 0.212819784 | −6.136888071 | 8.41536E−10 | 8.75259E−08 |
| Gpatch3 | 138.991251 | −1.310400191 | 0.413245403 | −3.170997627 | 0.001519164 | 0.02426745 |
| Cdca2 | 323.115498 | −1.318244413 | 0.393348159 | −3.351342527 | 0.000804208 | 0.014691224 |
| Cks1b | 4394.19378 | −1.335892505 | 0.153480564 | −8.703984861 | 3.2043E−18 | 1.30452E−15 |
| E130307A14Rik | 264.953883 | −1.338675837 | 0.341278106 | −3.922536531 | 8.76216E−05 | 0.002387228 |
| 2810408A11Rik | 203.964047 | −1.341778119 | 0.450592471 | −2.977808564 | 0.002903173 | 0.039222651 |
| Myh10 | 216.73725 | −1.343797559 | 0.42840337 | −3.136757679 | 0.001708273 | 0.026573335 |
| 1190002F15Rik | 1228.27547 | −1.34884084 | 0.23472775 | −5.746405535 | 9.11605E−09 | 7.59618E−07 |
| Cdk5rap1 | 951.050335 | −1.355335157 | 0.346557751 | −3.910849358 | 9.19721E−05 | 0.002482028 |
| Mgst1 | 1298.85228 | −1.356084626 | 0.204556733 | −6.629381513 | 3.37096E−11 | 4.95184E−09 |
| Filip1l | 438.713224 | −1.362386392 | 0.39023749 | −3.491172499 | 0.000480906 | 0.009747403 |
| Rad51ap1 | 603.668206 | −1.370712554 | 0.359460007 | −3.813254683 | 0.000137149 | 0.003464949 |
| Rel | 147.364104 | −1.371446027 | 0.403384072 | −3.399851706 | 0.000674224 | 0.012774823 |
| St3gal5 | 240.129897 | −1.385848745 | 0.48071569 | −2.882886438 | 0.003940495 | 0.049080515 |
| Cks2 | 858.501933 | −1.386695533 | 0.218859611 | −6.336004754 | 2.358E−10 | 2.77678E−08 |
| Syk | 796.292299 | −1.3879761 | 0.276813995 | −5.014111014 | 5.32792E−07 | 2.80139E−05 |
| Rad51b | 342.336113 | −1.390101944 | 0.288501115 | −4.818358995 | 1.44744E−06 | 6.85201E−05 |
| Aurka | 748.045968 | −1.400534823 | 0.334802291 | −4.183169775 | 2.87473E−05 | 0.00091843 |
| Cenph | 591.420387 | −1.402763895 | 0.480892634 | −2.917000171 | 0.003534156 | 0.0449259 |
| Cenpp | 432.231269 | −1.412769203 | 0.260241287 | −5.428689733 | 5.67693E−08 | 4.00448E−06 |
| Tnfaip8l1 | 134.655264 | −1.434229749 | 0.472322165 | −3.036549747 | 0.002393026 | 0.033878855 |
| Hmgb2 | 3511.98076 | −1.435623025 | 0.20931783 | −6.858579728 | 6.95485E−12 | 1.19397E−09 |
| Tpx2 | 767.408944 | −1.435848131 | 0.406073764 | −3.535929325 | 0.000406343 | 0.008455198 |
| Fbxo5 | 793.778266 | −1.436220114 | 0.405256547 | −3.543977578 | 0.000394139 | 0.00826652 |
| Uhrf1 | 674.686343 | −1.437131916 | 0.23901629 | −6.012694426 | 1.82465E−09 | 1.73329E−07 |
| Rgs12 | 307.004827 | −1.459687951 | 0.384612828 | −3.795213901 | 0.000147516 | 0.003681188 |
| Id1 | 120.574074 | −1.472625084 | 0.473505782 | −3.110046676 | 0.001870578 | 0.028348279 |
| Ppbp | 314.771376 | −1.478721468 | 0.39096551 | −3.782229967 | 0.00015543 | 0.003838332 |
| Kif20a | 309.657442 | −1.484165108 | 0.459997728 | −3.226461823 | 0.00125331 | 0.020813994 |
| Phldb2 | 139.379737 | −1.489358103 | 0.479001803 | −3.1092954 | 0.001875341 | 0.028367024 |
| Ms4a6b | 1391.95131 | −1.49464604 | 0.292119425 | −5.116558196 | 3.11161E−07 | 1.73872E−05 |
| Lat2 | 251.575335 | −1.502021246 | 0.442534927 | −3.394130392 | 0.000688469 | 0.012993371 |
| Rps4l | 813.40242 | −1.507130407 | 0.303837645 | −4.960314935 | 7.0379E−07 | 3.65996E−05 |
| Skint3 | 365.035435 | −1.512077455 | 0.428124322 | −3.531865345 | 0.000412639 | 0.008558514 |
| Sell | 195.438849 | −1.512954051 | 0.476809601 | −3.17307799 | 0.00150832 | 0.024161721 |
| Cenpk | 902.140906 | −1.528067944 | 0.301185506 | −5.073510886 | 3.90542E−07 | 2.10789E−05 |
| Tonsl | 231.793998 | −1.531495593 | 0.324921867 | −4.713427277 | 2.43585E−06 | 0.000108126 |
| Rrm2 | 1316.13494 | −1.56095271 | 0.329719521 | −4.734183485 | 2.19939E−06 | 9.91743E−05 |
| Bub1 | 469.412703 | −1.571756244 | 0.261190558 | −6.017661045 | 1.76955E−09 | 1.69264E−07 |
| F2rl2 | 267.25412 | −1.571818854 | 0.334642198 | −4.697013299 | 2.63993E−06 | 0.0001161 |
| Ccnb1 | 996.355168 | −1.592392545 | 0.355554768 | −4.478613953 | 7.51293E−06 | 0.000295723 |
| Chaf1a | 616.615661 | −1.597799219 | 0.254382117 | −6.281098831 | 3.36188E−10 | 3.86318E−08 |
| Fgd2 | 469.586661 | −1.600799015 | 0.360439553 | −4.441241256 | 8.94414E−06 | 0.000343518 |
| Lrr1 | 190.968489 | −1.611526769 | 0.478717076 | −3.366344861 | 0.000761714 | 0.014077386 |
| Nfam1 | 397.038846 | −1.621599684 | 0.280360487 | −5.783980842 | 7.29533E−09 | 6.30007E−07 |
| Ndc80 | 830.004808 | −1.62833525 | 0.343817771 | −4.73604155 | 2.17933E−06 | 9.85818E−05 |
| Apitd1 | 593.382583 | −1.63240114 | 0.306830247 | −5.320209312 | 1.03648E−07 | 6.71309E−06 |
| E2f1 | 322.888387 | −1.634637754 | 0.283638036 | −5.763111948 | 8.2577E−09 | 6.92141E−07 |
| Lgals1 | 785.131617 | −1.653771552 | 0.19789816 | −8.356679779 | 6.45081E−17 | 2.41888E−14 |
| Cenpm | 578.925418 | −1.674637719 | 0.257858852 | −6.494396866 | 8.33667E−11 | 1.11018E−08 |
| Akr1c12 | 310.766582 | −1.680290787 | 0.489627616 | −3.431772904 | 0.000599649 | 0.01165676 |
| Dscc1 | 475.644523 | −1.686392842 | 0.299564256 | −5.629486189 | 1.80747E−08 | 1.38466E−06 |
| Cenpa | 1449.48008 | −1.688770878 | 0.303580283 | −5.562847688 | 2.65408E−08 | 1.99042E−06 |
| Chek1 | 925.117971 | −1.689415257 | 0.327400206 | −5.160092215 | 2.46828E−07 | 1.42391E−05 |
| Ttk | 325.623539 | −1.691156691 | 0.478289108 | −3.535846132 | 0.000406471 | 0.008455198 |
| Hist1h2ae | 964.202552 | −1.693421442 | 0.329810006 | −5.134536276 | 2.82841E−07 | 1.59296E−05 |
| Fxyd1 | 104.476882 | −1.694206776 | 0.52449681 | −3.230156495 | 0.001237225 | 0.02066731 |
| Lmnb1 | 242.386896 | −1.694336491 | 0.478109875 | −3.54382241 | 0.000394371 | 0.00826652 |
| Tnfrsf13c | 367.563352 | −1.69831795 | 0.337128968 | −5.03759128 | 4.71427E−07 | 2.50648E−05 |
| Nrk | 206.03742 | −1.719182361 | 0.486681928 | −3.532455723 | 0.000411719 | 0.008551875 |
| Mc5r | 114.157746 | −1.73023846 | 0.522181558 | −3.313480599 | 0.000921425 | 0.016330081 |
| Il12rb2 | 146.901761 | −1.734990032 | 0.467101216 | −3.714377041 | 0.000203705 | 0.004805614 |
| Ckap2 | 731.108628 | −1.745959849 | 0.278334717 | −6.27287846 | 3.54434E−10 | 4.04026E−08 |
| Fam105a | 669.649301 | −1.746463368 | 0.265438275 | −6.57954609 | 4.71887E−11 | 6.68292E−09 |
| Mcm10 | 187.735365 | −1.747837713 | 0.570800162 | −3.062083421 | 0.002198022 | 0.031667961 |
| Spp1 | 169.284354 | −1.753828579 | 0.58104416 | −3.018408408 | 0.002541062 | 0.035672505 |
| Spic | 216.686065 | −1.759409073 | 0.542755966 | −3.241620883 | 0.00118852 | 0.020065431 |
| Rad54b | 400.362097 | −1.767009397 | 0.335211484 | −5.27132716 | 1.35441E−07 | 8.57732E−06 |
| Cenpn | 532.212876 | −1.774613113 | 0.410437755 | 4.323708266 | 1.53428E−05 | 0.000541138 |
| Fancd2 | 114.716017 | −1.805265566 | 0.47155417 | −3.828331253 | 0.000129015 | 0.00327689 |
| Tyrobp | 114.362098 | −1.815964396 | 0.51537146 | −3.52360295 | 0.000425722 | 0.008791461 |
| Ms4a6c | 473.589687 | −1.833809551 | 0.293272439 | −6.252921549 | 4.02845E−10 | 4.48448E−08 |
| Kif22 | 647.788325 | −1.83458771 | 0.385157914 | −4.76320918 | 1.90538E−06 | 8.67405E−05 |
| Ect2 | 456.386913 | −1.836475278 | 0.331525061 | −5.539476487 | 3.03377E−08 | 2.25147E−06 |
| Col4a2 | 421.959129 | −1.839550864 | 0.375443713 | −4.899671515 | 9.5997E−07 | 4.85057E−05 |
| Oas2 | 194.67636 | −1.85717158 | 0.499329414 | −3.719331422 | 0.000199751 | 0.004727989 |
| Mfsd2b | 202.515868 | −1.858589216 | 0.450554544 | −4.125114795 | 3.7055E−05 | 0.001157887 |
| Spc25 | 1410.98784 | −1.86019522 | 0.225676194 | −8.242762286 | 1.6828E−16 | 5.99454E−14 |
| Mis18bp1 | 547.785963 | −1.862446171 | 0.47671473 | −3.906835793 | 9.35127E−05 | 0.002518831 |
| Polq | 194.621428 | −1.865204815 | 0.588995634 | −3.166754908 | 0.001541502 | 0.024596706 |
| Mybl2 | 111.365472 | −1.868110217 | 0.479667552 | −3.894593684 | 9.83635E−05 | 0.002629608 |
| Igf1 | 310.276766 | −1.871823957 | 0.444480367 | −4.211263527 | 2.53946E−05 | 0.000829379 |
| Shcbp1 | 759.406293 | −1.879617068 | 0.430615108 | −4.36495848 | 1.27147E−05 | 0.000460997 |
| Ebi3 | 1332.19493 | −1.885787615 | 0.255613038 | −7.377509508 | 1.61278E−13 | 3.83008E−11 |
| Kif15 | 325.154189 | −1.899696936 | 0.515773523 | −3.683199796 | 0.000230324 | 0.005319115 |
| Kcnk12 | 143.472785 | −1.908747913 | 0.53383705 | −3.575525365 | 0.000349525 | 0.007511894 |
| Ptn | 292.223688 | −1.914600669 | 0.564520166 | −3.391554077 | 0.000694974 | 0.013064039 |
| Cdk1 | 1828.8631 | −1.917561096 | 0.292071319 | −6.565386509 | 5.18981E−11 | 7.17957E−09 |
| 5430427O19Rik | 523.811955 | −1.939514952 | 0.263869854 | −7.350271058 | 1.97805E−13 | 4.62054E−11 |
| Ugt1a7c | 99.8099714 | −1.94275864 | 0.573943586 | −3.384929615 | 0.000711965 | 0.013330868 |
| Slc35d3 | 105.180173 | −1.946697005 | 0.580350066 | −3.354349591 | 0.000795518 | 0.014551135 |
| Ctss | 430.400784 | −1.948239277 | 0.396665317 | −4.911544306 | 9.03619E−07 | 4.61493E−05 |
| Spc24 | 1183.00222 | −1.951846959 | 0.63369982 | −3.080081291 | 0.002069441 | 0.030212567 |
| Tcf19 | 416.026607 | −1.954834234 | 0.297540207 | −6.569983446 | 5.03209E−11 | 7.02963E−09 |
| Traf3ip2 | 89.3405019 | −1.961052849 | 0.552371774 | −3.550240875 | 0.000384879 | 0.008112631 |
| Cenpe | 913.15855 | −1.966531631 | 0.312388527 | −6.295146782 | 3.07109E−10 | 3.55772E−08 |
| Knstrn | 712.578109 | −1.984545896 | 0.381066119 | −5.207878094 | 1.91012E−07 | 1.14359E−05 |
| Fam46a | 78.5934022 | −1.995618468 | 0.65718816 | −3.03660137 | 0.002392616 | 0.033878855 |
| Arhgef39 | 428.769225 | −1.996565719 | 0.367098714 | −5.438770669 | 5.36495E−08 | 3.82226E−06 |
| Tspan2 | 130.586566 | −2.002536546 | 0.447012208 | −4.479825183 | 7.47042E−06 | 0.000294864 |
| Mthfd2 | 175.111948 | −2.027600226 | 0.59328927 | −3.417557552 | 0.000631857 | 0.012154542 |
| Prc1 | 1519.17838 | −2.033436011 | 0.53807855 | −3.779069079 | 0.000157416 | 0.00387395 |
| Pf4 | 2876.08509 | −2.039912637 | 0.203189563 | −10.03945579 | 1.02237E−23 | 6.33378E−21 |
| Sik1 | 132.006502 | −2.040234624 | 0.65282859 | −3.125222541 | 0.001776706 | 0.027251111 |
| 9030619P08Rik | 748.889835 | −2.045989377 | 0.259207156 | −7.893259607 | 2.94394E−15 | 8.73922E−13 |
| Cep55 | 480.789247 | −2.046266008 | 0.482077611 | −4.244681687 | 2.18904E−05 | 0.000735651 |
| Hist1h1c | 387.223025 | −2.047839858 | 0.557733619 | −3.67171673 | 0.000240927 | 0.005498257 |
| Hist1h2ab | 58.3626725 | −2.049988359 | 0.682906517 | −3.001857953 | 0.002683374 | 0.036906747 |
| E2f8 | 192.573953 | −2.05937341 | 0.374646601 | −5.496842643 | 3.86651E−08 | 2.79665E−06 |
| Jakmip1 | 106.115218 | −2.063319418 | 0.493199043 | −4.183543031 | 2.87001E−05 | 0.00091843 |
| Endod1 | 73.1491809 | −2.073376957 | 0.667272333 | −3.107242507 | 0.001888414 | 0.028433849 |
| Cdc20 | 1130.90773 | −2.073876595 | 0.305396162 | −6.790774917 | 1.11533E−11 | 1.84794E−09 |
| Gata1 | 246.593889 | −2.090842731 | 0.520359702 | −4.018071968 | 5.86763E−05 | 0.001692466 |
| Eldr | 252.593473 | −2.103586823 | 0.572559867 | −3.674003267 | 0.00023878 | 0.005461271 |
| Top2a | 1964.10317 | −2.130786855 | 0.708769167 | −3.006319905 | 0.002644306 | 0.036694475 |
| Csf2rb | 146.432495 | −2.135381981 | 0.68976142 | −3.095826933 | 0.001962649 | 0.02919184 |
| Spdl1 | 204.723773 | −2.145619548 | 0.420953753 | −5.0970434 | 3.44999E−07 | 1.9128E−05 |
| Ncaph | 339.446634 | −2.154028346 | 0.423984647 | −5.080439498 | 3.76563E−07 | 2.04801E−05 |
| Flt3 | 242.788593 | −2.167128028 | 0.435551359 | −4.975596983 | 6.50469E−07 | 3.39506E−05 |
| Hist1h3e | 189.631676 | −2.201870159 | 0.411688345 | −5.348390809 | 8.87397E−08 | 5.88117E−06 |
| Shisa8 | 399.782638 | −2.213397119 | 0.31310189 | −7.069255054 | 1.55768E−12 | 2.95938E−10 |
| Ccne2 | 302.494123 | −2.216104628 | 0.335972943 | −6.596080653 | 4.22169E−11 | 6.07625E−09 |
| Cd69 | 926.237776 | −2.321355867 | 0.391824911 | −5.924472395 | 3.13302E−09 | 2.88015E−07 |
| Pbk | 2042.59738 | −2.339678883 | 0.672181914 | −3.480722754 | 0.000500063 | 0.010064117 |
| Fanci | 123.527916 | −2.342273894 | 0.771400208 | −3.036392614 | 0.002394274 | 0.033878855 |
| Plekhg5 | 76.174811 | −2.352237975 | 0.609099652 | −3.861827811 | 0.000112542 | 0.002931644 |
| Cdca3 | 570.135189 | −2.437334091 | 0.814813099 | −2.991279956 | 0.002778107 | 0.037986228 |
| BC051537 | 58.6197595 | −2.465480929 | 0.79199045 | −3.11301851 | 0.001851844 | 0.02819116 |
| Haao | 80.8791377 | −2.521699054 | 0.627416659 | −4.019177713 | 5.84016E−05 | 0.001687975 |
| Rgs2 | 2422.25068 | −2.525257621 | 0.71895024 | −3.512423364 | 0.00044404 | 0.009090699 |
| Nek2 | 172.678666 | −2.529895668 | 0.682032374 | −3.709348362 | 0.000207793 | 0.004885887 |
| Depdc1a | 73.2615112 | −2.569613295 | 0.660201451 | −3.892165478 | 9.93534E−05 | 0.002644342 |
| Gm16712 | 181.966183 | −2.591964331 | 0.418944852 | −6.186886698 | 6.1364E−10 | 6.62406E−08 |
| Ifi27l2a | 57.2077771 | −2.600211161 | 0.802103251 | −3.241741205 | 0.001188019 | 0.020065431 |
| Mmp2 | 80.2781721 | −2.658471281 | 0.766932586 | −3.46636892 | 0.000527539 | 0.010527874 |
| Srl | 50.4876306 | −2.698631669 | 0.782848164 | −3.44719678 | 0.000566436 | 0.011163409 |
| Dlgap5 | 321.741655 | −2.724411017 | 0.496410972 | −5.488216758 | 4.06012E−08 | 2.92185E−06 |
| Hist2h2ac | 50.5959157 | −2.746506165 | 0.767968277 | −3.576327625 | 0.000348455 | 0.007505605 |
| Esco2 | 637.928813 | −2.760316112 | 0.863193535 | −3.197795163 | 0.001384826 | 0.022577102 |
| Casc5 | 661.740752 | −2.808703621 | 0.840419283 | −3.342026627 | 0.000831691 | 0.015115768 |
| Rnase6 | 1274.52637 | −2.878566889 | 0.193106285 | −14.90664526 | 2.98371E−50 | 7.08582E−47 |
| Cdc25c | 96.5994252 | −2.965136679 | 0.956301634 | −3.100629105 | 0.0019311 | 0.028863007 |
| Satb1 | 97.4800299 | −3.081374488 | 0.600080678 | −5.13493369 | 2.82244E−07 | 1.59296E−05 |
| C1qc | 62.6282347 | −3.139675229 | 1.044876002 | −3.004830452 | 0.002657289 | 0.036781666 |
| Cd86 | 229.348161 | −3.168116264 | 0.444967209 | −7.119887043 | 1.08016E−12 | 2.16777E−10 |
| Fam83d | 147.912773 | −3.205415997 | 0.852442789 | −3.760271114 | 0.000169729 | 0.004106066 |
| Cd52 | 61.5410577 | −3.325556149 | 0.7990795 | −4.16173378 | 3.1584E−05 | 0.001000091 |
| Hist1h1b | 105.746292 | −3.344835581 | 1.033353558 | −3.236874305 | 0.001208466 | 0.020271062 |
| Sdc1 | 32.4342957 | −3.365675127 | 1.151562185 | −2.92270376 | 0.003470065 | 0.044345251 |
| Bank1 | 107.574721 | −3.459718348 | 0.866478636 | −3.992848994 | 6.52841E−05 | 0.001853055 |
| Ncf4 | 194.201903 | −3.560786521 | 0.489531261 | −7.273869526 | 3.49333E−13 | 8.02847E−11 |
| 3830403N18Rik | 54.7362387 | −3.578678499 | 1.183830287 | −3.022965824 | 0.002503105 | 0.035278679 |
| Troap | 86.4361274 | −3.653561412 | 0.965725469 | −3.78322984 | 0.000154806 | 0.003829576 |
| Peg3 | 18.3926769 | −3.78447716 | 1.302541557 | −2.905455983 | 0.003667184 | 0.046079108 |
| Rgs7bp | 232.876383 | −3.985539216 | 0.555792542 | −7.170911658 | 7.45E−13 | 1.60841E−10 |
| Gm13710 | 34.8124987 | −4.083357874 | 1.394474161 | −2.92824205 | 0.003408846 | 0.043798594 |
| Gpr182 | 24.032774 | −4.233526389 | 1.443811511 | −2.932187725 | 0.003365832 | 0.043467654 |
| Slc4a10 | 32.9396399 | −4.654635788 | 1.126548441 | −4.131767101 | 3.59985E−05 | 0.001127347 |
| C1qa | 46.335912 | −4.699173141 | 1.258301986 | −3.734535266 | 0.000188062 | 0.004488605 |
| Ccdc36 | 17.8534849 | −4.734731238 | 1.569082161 | −3.017516454 | 0.002548552 | 0.035706963 |
| Cnr2 | 66.1318567 | −4.825670538 | 1.079331873 | −4.47097937 | 7.78622E−06 | 0.000303961 |
| Bmp8a | 19.2871825 | −4.849544624 | 1.535937466 | −3.157384158 | 0.001591915 | 0.025231583 |
| Rhag | 110.605017 | −4.920372499 | 0.910563932 | −5.403654072 | 6.52968E−08 | 4.51657E−06 |
| Tgm5 | 42.1734926 | −4.963606735 | 1.126962708 | −4.404410813 | 1.06072E−05 | 0.000397741 |
| Cd83 | 42.949235 | −5.28884643 | 1.221415625 | −4.330095607 | 1.49045E−05 | 0.000532265 |
| Boll | 13.8608298 | −5.388852262 | 1.793517848 | −3.004627063 | 0.002659067 | 0.036781666 |
| Dqx1 | 23.4675845 | −5.588004729 | 1.447772794 | −3.859724917 | 0.000113515 | 0.00295159 |
| Mpp3 | 17.9392293 | −5.745460923 | 1.562823268 | −3.676334387 | 0.000236609 | 0.005422844 |
| Pkib | 58.4985381 | −5.891021615 | 1.065924882 | −5.526676144 | 3.26355E−08 | 2.38473E−06 |
| Ptprm | 11.2774827 | −6.003169777 | 2.088716478 | −2.874095092 | 0.004051869 | 0.049955922 |
| Tmem74 | 32.6451651 | −6.052988958 | 1.348018336 | −4.490286812 | 7.11273E−06 | 0.00028231 |
| Sparc | 22.0613594 | −6.102444267 | 1.814660624 | −3.362857046 | 0.000771403 | 0.014219562 |
| Ms4a4b | 24.7297865 | −6.197099463 | 1.505506769 | −4.116288012 | 3.85023E−05 | 0.001187488 |
| Hmga2-ps1 | 24.2841334 | −6.212827366 | 2.033228581 | −3.055646288 | 0.00224576 | 0.032233329 |
| Eid3 | 15.3514956 | −6.453962659 | 1.81430169 | −3.55727093 | 0.000374728 | 0.007933869 |
| Omd | 9.11205406 | −6.675950826 | 2.19784526 | −3.037498111 | 0.002385509 | 0.033878855 |
| Scimp | 9.13079789 | −6.679448082 | 2.168865643 | −3.07969657 | 0.002072116 | 0.030220653 |
| Fam46d | 18.2675049 | −6.705815358 | 2.135614704 | −3.139993064 | 0.001689518 | 0.026513157 |
| Mrap | 9.99586803 | −6.809975134 | 2.105520652 | −3.234342597 | 0.001219231 | 0.020414598 |
| Pde10a | 20.0288123 | −6.839618642 | 2.094893988 | −3.264899647 | 0.001095029 | 0.018798871 |
| Ret | 10.3529151 | −6.859671738 | 2.22525686 | −3.082642665 | 0.002051714 | 0.030077024 |
| Oosp1 | 10.5108593 | −6.881949553 | 2.150018671 | −3.200878972 | 0.001370091 | 0.022362454 |
| Grin3a | 10.6050638 | −6.895525197 | 2.079677439 | −3.315670531 | 0.000914235 | 0.01622284 |
| AW822252 | 10.7174176 | −6.91039281 | 2.101156458 | −3.28885209 | 0.001005969 | 0.01758779 |
| Synpo2 | 11.0704154 | −6.958326367 | 2.212788988 | −3.144595532 | 0.001663166 | 0.026186135 |
| Map3k15 | 11.5848097 | −7.02219576 | 2.059781878 | −3.409193874 | 0.000651552 | 0.012444985 |
| Dsc1 | 12.2572573 | −7.104654469 | 2.46404664 | −2.883327918 | 0.003934976 | 0.049054654 |
| Tespa1 | 12.8590521 | −7.173808356 | 2.027063657 | −3.539014836 | 0.000401623 | 0.0083911 |
| 1700024F13Rik | 13.0182382 | −7.191580646 | 2.005836362 | −3.585327688 | 0.000336655 | 0.007290274 |
| Ccr1 | 13.0656105 | −7.196812293 | 2.422482685 | −2.970841583 | 0.002969849 | 0.039808186 |
| Kcne1l | 13.3263498 | −7.225159302 | 2.415857781 | −2.990722119 | 0.002783186 | 0.037986228 |
| Alox5 | 13.4142311 | −7.234776428 | 2.40822688 | −3.004192208 | 0.00266287 | 0.036781666 |
| Tagln | 13.5699606 | −7.250958044 | 1.911370325 | −3.793591409 | 0.000148484 | 0.003698859 |
| Wdr86 | 13.8440958 | −7.280894681 | 2.041400847 | −3.566616861 | 0.00036162 | 0.007702119 |
| Tnfrsf11b | 14.186881 | −7.31486422 | 1.937217061 | −3.775965207 | 0.000159389 | 0.003902298 |
| Arhgdig | 14.637809 | −7.360486736 | 1.891990841 | −3.890339517 | 0.000100104 | 0.002651269 |
| Spta1 | 15.8280528 | −7.472874745 | 2.333779431 | −3.202048422 | 0.001364541 | 0.022297407 |
| Hesx1 | 15.8995739 | −7.479615413 | 1.830617913 | −4.085841922 | 4.39173E−05 | 0.001325799 |
| Cd209f | 17.0100367 | −7.576639097 | 2.281076125 | −3.321519617 | 0.000895287 | 0.015974093 |
| Kynu | 34.4775669 | −7.628089501 | 1.500093396 | −5.085076385 | 3.67478E−07 | 2.0217E−05 |
| Pdlim4 | 18.0352112 | −7.661070945 | 1.866020799 | −4.105565676 | 4.03327E−05 | 0.001235914 |
| Tmeff1 | 18.3048097 | −7.682784802 | 1.78299908 | −4.308911255 | 1.6406E−05 | 0.000571563 |
| Il1bos | 19.1441133 | −7.747206586 | 1.754351389 | −4.415994785 | 1.00547E−05 | 0.000379018 |
| D430019H16Rik | 21.489606 | −7.914523693 | 1.691932862 | −4.677800088 | 2.89969E−06 | 0.000126354 |
| Siglecg | 28.6042978 | −8.327026823 | 1.717317057 | −4.848858158 | 1.24174E−06 | 5.99782E−05 |
| Plagl1 | 41.7727125 | −8.873759651 | 1.833584027 | −4.839570766 | 1.3012E−06 | 6.22174E−05 |
| Flrt3 | 46.3705126 | −9.023212856 | 1.584660515 | −5.69409837 | 1.24026E−08 | 9.87285E−07 |
| Cxcl2 | 70.5291856 | −9.629234786 | 1.588972462 | −6.060038808 | 1.36089E−09 | 1.36558E−07 |

TABLE 2
List of the 54 potential marker genes for old HSC heterogeneity

| Membrane located proteins | Non-membrane located proteins |
|---|---|
| Alcam | Ar |
| Art4 | B3gnt8 |
| Cd150 | Bmp4 |
| Cd38 | Clu |
| Chrm3 | Cyp26b1 |
| Cldn5 | Dnm3 |
| Efna1 | Fads2 |
| Ehd3 | Gadd45g |
| Enpp5 | Gda |
| Epcam | Gpx3 |
| Gpr183 | Mt1 |
| Itgb3 | Mt2 |
| Jam2 | Ntf3 |
| Kcnb2 | Nupr1 |
| Klrb1c | Oxr1 |
| Lpar6 | Pam |
| Neo1 | Plcl1 |
| Npdc1 | Rbm19 |
| Nrg4 | Rdh10 |
| Plscr2 | Rorc |
| Procr | Sbspon |
| Prtn3 | Smtnl1 |
| Ptger4 | Sox18 |
| Ramp2 | Sult1a1 |
| Selp | Tox |
| Tm4sf1 | Vwf |
| Slamf1 | Xdh |
| | Zg16 |

TABLE 3A
List of the CD150 correlated genes based on bulk RNA-seq
CD150 positively correlated genes (CD150 feature genes)

Slamf1
Sbspon
Ehd3
Clu
Dpy19l2
Kcnb2
B3gnt8
Rbpjl
Cited2
Terf1
Fam63a
Gipc2
Enpp5
Ctsw
Ccdc92
Oaf
Dnm3os
Sdf4
Selp
Arpin
Hfe
4631405J19Rik
Sla2
Jam2
Ccnd2
Eid2
Matn4
Dhx40
Prkag2
Krt18
Slc14a1
Mycn
Bace2
Ncoa7
Epdr1
Pde9a
Ces2g
Oxr1
Krt80
Gm6654
Fam213a
Efcab14
Fez2
Trim47
Rad9b
Ppap2a
Trib3
Tacstd2
Gem
Clip3
Tacr1
Efhd1
Hpcal1
Cmas
Art4
Echdc2
Lrrc73
Fkbp10
B4galt4
Rab36
Sil1
Tc2n
Nxpe2
Gstm2
Hoxb7
Rapgef4
Fam131a
Eef2kmt
Rarb
Bcat2
Relb
Armc2
Smyd2
Phactr1
Fndc4
4930512B01Rik
Tnni3
Rae1
Eid1
Gfod1
Mboat2
Rtp4
Nufip1
Slc25a4
Slc9a3r2
Zg16
Taf11
Rbm19
Nova2
Pebp1
Npdc1
Mylk
Ctsb
4921509O07Rik
Skap1
Lrrc42
Igtp
Dnm3
Fhl1
Ryr3
Isca1
Gimap5
Veph1
Mllt3
Ttc14
Ccdc64
Erc2
Anpep
Exoc3l
Ctsh
Rnf128
Kitl
Cd38
Shisa5
Fads3
Slc24a5
Ap2m1
Dyrk3
Lmtk3
Tnfsf4
Pygm
Irgm1
Rpl3
Slamf8
Zfp622
Stx1a
Klrb1c
Ptger4
AA986860
Mtg1
Pepd

TABLE 3B
List of the CD150 correlated genes based on bulk RNA-seq
CD150 negatively correlated genes

Pdik1l
Serpinb1a
Elf1
1700097N02Rik
BC016579
1110002L01Rik
Smap2
Slc38a2
Usp6nl
Ptpn22
Lrr1
Zeb1
Mlec
Heatr6
Iqgap2
Col4a2
Wfdc17
Slmap
Ssh2
Fxr2
Pitpnc1
Gpr171
Runx2
Gng2
Chsy1
Smek2
Ctla2a
Ccl3
Ikzf5
Kif15
Mybpc3
Eed
Clnk
St8sia4
Dpp4
Naa50
Atad2
B3galt2
Myb
Heatr5b
Arhgap18
Akr1c12
Zcwpw1
Fabp5
Tuba8
Kdm3a
Cd1d1
Sppl2a
Syk
Reep5
Clspn
Fam105a
Ptpre
Rian
Pigu
Shisa8
Agpat1
Nkap
Celf2
Fxyd1
Slbp
Wipf1
Rnase6
Hmgb2
Rnu11
Galnt6
Ppp3cb
Dennd5a
Prss57
Camk1d
Rnase4
Cux1
Ccdc112
Tsg101
Spdl1
Hspa4l
Mybl1
Slc12a6
I730030J21Rik
Nkg7
Haus6
Golm1
Lat
Fam173a
Cdh18
Prim1
Smad6
Ppfia3
Kcnab2
Ndc80
Lrrc36
Tmem65
Zc3hc1
Cmss1
Ly96
Rbl1
Cd27
Cggbp1
Kif20b
Eef1e1
Cdca8
Bfsp2
Cmtm7

What is claimed is:

1. A method for characterizing hematopoietic stem cell (HSC) functionality, the method comprising detecting the level of CD150 polypeptide or polynucleotide in an HSC, wherein an alteration in the level of CD150 relative to a reference is indicative of HSC functionality.

2. A method for selecting a hematopoietic stem cell (HSC) for engraftment in a subject in need thereof, the method comprising detecting the level of CD150 polypeptide or polynucleotide expression in an HSC, wherein detection of a reduced level of CD150 expression relative to a reference selects the HSC for engraftment in a subject.

3. The method of claim 1, wherein the HSC is present in a population marked by heterogeneity in HSC functionality.

4. The method of claim 1, wherein a reduction in the level of the CD150 polypeptide or polynucleotide relative to the reference indicates that the HSC has increased self-renewal capacity and is capable of balanced differentiation.

5. The method of claim 2, wherein a reduction in the level of CD150 polypeptide or polynucleotide relative to the reference indicates that the HSC should be selected for engraftment; and
wherein an increase in the level of CD150 polypeptide or polynucleotide relative to the reference indicates that the HSC should not be selected for engraftment.

6. The method of claim 1, wherein the method further comprises detecting the expression of one or more markers selected from the group consisting of Sbspon, Ehd3, Clu, Dpy19l2, Kcnb2, Selp, B3gnt8, Jam2 and Enpp5, wherein expression of said one or more markers positively correlates with CD150 expression; and
wherein the method further comprises detecting the expression of one or more markers selected from the group consisting of Serpinb1a, Usp6nl, Lrr1, Col4a2, Gng2, Kif15, Rnase6 and Arhgap30, wherein expression of said one or more markers negatively correlates with CD150 expression.

7. The method of claim 1, further comprising characterizing the level of one or more of 332 HSC aging marker genes (Tables 1A, 1B), wherein detecting an increase in one or more of said genes is indicative of HSC functional decline.

8. The method of claim 1, wherein levels of CD150 polypeptide are detected using FACS analysis, and wherein levels of CD150 polynucleotide are detected using RNA-seq analysis.

9. A method of rejuvenating blood components of an aging or aged subject or alleviating aging-related hematopoietic cell functional decline, the method comprising:
administering to the subject an effective amount of hematopoietic stem cells (HSCs) expressing low levels of CD150 polypeptide (CD150^{low} HSCs) or a composition thereof comprising a physiologically acceptable carrier, excipient or diluent; wherein the CD150^{low} HSCs differentiate into hematopoietic cell types which rejuvenate blood components in the aged subject.

10. The method of claim 9, wherein CD150^{low} HSCs have a mean fluorescence intensity of less than about 4×10³ cell surface expressed CD150 proteins per cell or wherein CD150^{low} HSCs comprise the lowest 25% subpopulation of a Long-Term-HSC population of bone marrow which expresses a Lin⁻Sca-1⁺c-Kit⁺CD48⁻CD34⁻CD150⁺ cell surface biomarker signature based on flow cytometry or fluorescence activated cell sorting (FACS) analysis.

11. The method of claim 10, wherein CD150^{low} HSCs express marker genes Rnase6 and Arhgap30.

12. The method of claim 9, wherein, following administration of the CD150^{low} HSCs, the subject exhibits increased numbers of naïve T cells and a decrease in central memory T cell (Tcm) and effector memory T cell (Tem) ratio in the CD8⁺ and CD4⁺ subgroups of T cells.

13. A method of improving physical health and aging-related hematopoietic cell functional decline of a selected subject, the method comprising:
selecting the subject by detecting hematopoietic stem cells (HSC) expressing high levels of CD150 polypeptide (CD150^{high} HSCs) in a sample obtained from the subject; and
administering to the subject an effective amount of hematopoietic stem cells (HSCs) expressing low levels of CD150 polypeptide (CD150^{low} HSCs) or a composition thereof comprising a physiologically acceptable carrier, excipient or diluent; wherein the CD150^{low} HSCs differentiate into hematopoietic cell types which improve physical health and aging-related hematopoietic cell functional decline in the subject.

14. The method of claim 13, wherein the subject having CD150^{high} HSCs is selected by further detecting in cells of the sample one or more age-related genes selected from Sbspon, Ehd3, Clu, Dpy19l2, Kcnb2, Selp, B3gnt8, Jam2, Enpp5, Gpr183, or Ramp2; and
wherein CD150^{low} HSCs express one or more genes selected from Serpinb1a, Usp6nl, Lrr1, Col4a2, Gng2, Kif15, Rnase6, or Arhgap30.

15. The method of claim 13, wherein the CD150^{low} HSCs administered to the subject dilute or reduce the numbers of CD150^{high} HSCs in the subject, thereby rejuvenating the blood with CD150^{low} HSCs capable of differentiation into activated or activatable hematopoietic cells and cell types.

16. A composition comprising an effective amount of hematopoietic stem cells (HSCs) expressing a low abundance of the CD150 glycoprotein (CD150^{low} HSCs), wherein said CD150^{low} HSCs have a mean fluorescence intensity of less than about 4×10³ cell surface expressed CD150 proteins per cell or wherein CD150^{low} HSCs comprise the lowest 25% subpopulation of a Long-Term-HSC population of bone marrow which expresses a Lin⁻Sca-1⁺c-Kit⁺CD48⁻CD34⁻CD150⁺ cell surface biomarker signature based on flow cytometry or fluorescence activated cell sorting (FACS) analysis.

17. Use of a composition comprising an effective amount of hematopoietic stem cells expressing a low abundance of the CD150 glycoprotein (CD150^{low} HSCs), wherein said CD150^{low} HSCs have a mean fluorescence intensity of less than about 4×10³ cell surface expressed CD150 proteins per cell or wherein CD150^{low} HSCs comprise the lowest 25% subpopulation of a Long-Term-HSC population which expresses a Lin⁻Sca-1⁺c-Kit⁺CD48-CD34⁻CD150⁺ cell surface biomarker signature based on flow cytometry or fluorescence activated cell sorting (FACS) analysis and/or express marker genes Rnase6 and Arhgap30, in the manufacture of a medicament for treating an aging or aged subject having hematopoietic stem cells expressing a high abundance of the CD150 glycoprotein (CD150^{high} HSCs); wherein the CD150^{high} HSCs have a mean fluorescence intensity of greater than about 4×10³ cell surface expressed CD150 proteins per cell or wherein CD150^{high} HSCs comprise the highest 25% subpopulation of a Long-Term-HSC population which expresses a Lin⁻Sca-1⁺c-Kit⁺CD48⁻CD34⁻CD150⁺ cell surface biomarker signature based on flow cytometry or fluorescence activated cell sorting (FACS) analysis; and/or
wherein the CD150^{high} HSCs express a CD150 mRNA level of between about 0.3 to about 2 RNA Units per cell; and/or
wherein the CD150^{high} HSCs express one or more age-related marker genes selected from the group consisting of Sbspon, Ehd3, Clu, Dpy19l2, Kcnb2, Selp, B3gnt8, Jam2, Enpp5, Gpr183, and Ramp2.

18. A method of reducing or eliminating dysfunctional or defective CD150-expressing hematopoietic stem cells (HSCs) in a subject, the method comprising:
contacting a sample containing CD150-expressing hematopoietic stem cells (HSCs) obtained from a subject in need thereof with an anti-CD150 antibody conjugated to a cellular toxin in an amount effective to bind to high CD150-expressing HSCs (CD150^{high} HSCs); wherein the CD150^{high} HSCs express one or more age-related marker genes selected from the group consisting of Sbspon, Ehd3, Clu, Dpy19l2, Kcnb2, Selp, B3gnt8, Jam2, Enpp5, Gpr183, and Ramp2 which correlate with high expression of CD150 on the cell surface;
culturing the sample for a time sufficient for the anti-CD150 antibody-toxin conjugate to cause cytolysis of the high CD150-expressing HSCs; and
obtaining a sample in which dysfunctional or defective CD150-expressing hematopoietic stem cells (HSCs) are reduced or eliminated.

19. A method of reducing or eliminating dysfunctional or defective CD150-expressing hematopoietic stem cells (HSCs) in an old, aged or aging subject, the method comprising:
administering to a subject in need thereof an effective amount of an anti-CD150 antibody conjugated to a cellular toxin in an amount effective to bind to high CD150-expressing HSCs (CD150^{high} HSCs) in the subject; wherein the CD150^{high} HSCs express one or more age-related marker genes selected from the group consisting of Sbspon, Ehd3, Clu, Dpy19l2, Kcnb2, Selp, B3gnt8, Jam2, Enpp5, Gpr183, and Ramp2 which correlate with high expression of CD150 on the cell surface; thereby reducing or eliminating dysfunctional or defective CD150-expressing hematopoietic stem cells (HSCs) in the subject.

20. A kit or article of manufacture comprising a composition comprising an effective amount of hematopoietic stem cells (HSCs) expressing a low abundance of the CD150 glycoprotein (CD150^{low} HSCs), wherein said CD150^{low} HSCs have a mean fluorescence intensity of less than about 4×10³ cell surface expressed CD150 proteins per cell based on flow cytometry or fluorescence activated cell sorting (FACS) analysis and/or wherein CD150^{low} HSCs express one or more genes selected from Serpinb1a, Usp6nl, Lrr1, Col4a2, Gng2, Kif15, Rnase6, or Arhgap30, which correlate with CD150^{low} expressing HSCs, and instructions for use.


SEQUENCE LISTING (5 sequences)

SEQ ID NO: 1 - protein, 335 aa, Homo sapiens
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 2 - DNA, 1364 bp, Homo sapiens
  source 1..1364: mol_type = genomic DNA
ggccaggatcccttccttctcctcattggctgatggatcccaaggggctcctctccttgaccttcgtgctgtttctctccctggcttttggggcaagctacggaacaggtgggcgcatgatgaactgcccaaagattctccggcagttgggaagcaaagtgctgctgcccctgacatatgaaaggataaataagagcatgaacaaaagcatccacattgtcgtcacaatggcaaaatcactggagaacagtgtcgagaacaaaatagtgtctcttgatccatccgaagcaggccctccacgttatctaggagatcgctacaagttttatctggagaatctcaccctggggatacgggaaagcaggaaggaggatgagggatggtaccttatgaccctggagaaaaatgtttcagttcagcgcttttgcctgcagttgaggctttatgagcaggtctccactccagaaattaaagttttaaacaagacccaggagaacgggacctgcaccttgatactgggctgcacagtggagaagggggaccatgtggcttacagctggagtgaaaaggcgggcacccacccactgaacccagccaacagctcccacctcctgtccctcaccctcggcccccagcatgctgacaatatctacatctgcaccgtgagcaaccctatcagcaacaattcccagaccttcagcccgtggcccggatgcaggacagacccctcagaaacaaaaccatgggcagtgtatgctgggctgttagggggtgtcatcatgattctcatcatggtggtaatactacagttgagaagaagaggtaaaacgaaccattaccagacaacagtggaaaaaaaaagccttacgatctatgcccaagtccagaaaccaggtcctcttcagaagaaacttgactccttcccagctcaggacccttgcaccaccatatatgttgctgccacagagcctgtcccagagtctgtccaggaaacaaattccatcacagtctatgctagtgtgacacttccagagagctgacaccagagaccaacaaagggactttctgaaggaaaatggaaaaaccaaaatgaacactgaacttggccacaggccccaagtttcctctggcagacatgctgcacgtctgtacccttctcagatcaactccctggtgatgtttcttccacatacatctgtgaaatgaacaaggaagtgaggcttcccaagaatttagcttgctgtgcagtggctgcaggcgcagaacagagcgttacttgataacagcgttccatctttgtgttgtagcagatgaaatggacagtaatgtgagttcagactttgggcatcttgctcttggctgga

SEQ ID NO: 3 - protein, 343 aa, Mus musculus
MDPKGSLSWRILLFLSLAFELSYGTGGGVMDCPVILQKLGQDTWLPLTNEHQINKSVNKSVRILVTMATSPGSKSNKKIVSFDLSKGSYPDHLEDGYHFQSKNLSLKILGNRRESEGWYLVSVEENVSVQQFCKQLKLYEQVSPPEIKVLNKTQENENGTCSLLLACTVKKGDHVTYSWSDEAGTHLLSRANRSHLLHITLSNQHQDSIYNCTASNPVSSISRTFNLSSQACKQESSSESSPWMQYTLVPLGVVIIFILVFTAIIMMKRQGKSNHCQPPVEEKSLTIYAQVQKSGPQEKKLHDALTDQDPCTTIYVAATEPAPESVQEPNPTTVYASVTLPES

SEQ ID NO: 4 - DNA, 1282 bp, Mus musculus
  source 1..1282: mol_type = genomic DNA
ctcacagctggggaccctgtctgcgattgctggctaatggatcccaaaggatccctttcctggagaatacttctgtttctctccctggcttttgagttgagctacggaacaggtggaggtgtgatggattgcccagtgattctccagaagctgggacaggacacgtggctgcccctgacgaatgaacatcagataaataagagcgtgaacaaaagtgtccgcatcctcgtcaccatggcgacgtccccaggaagcaaatccaacaagaaaattgtgtcttttgatctctctaaagggagctatccagatcacctggaggatggctaccactttcaatcgaaaaacctgagcctgaagatcctcgggaacaggcgggagagtgaaggatggtacttggtgagcgtggaggagaacgtttctgttcagcaattctgcaagcagctgaagctttatgaacaggtctcccctccagagattaaagtgctaaacaaaacccaggagaacgagaatgggacctgcagcttgctgttggcctgcacagtgaagaaaggggaccatgtgacttacagctggagtgatgaggcaggcacccacctgctgagccgagccaaccgctcccacctcctgcacatcactcttagcaaccagcatcaagacagcatctacaactgcaccgcaagcaaccctgtcagcagtatctctaggaccttcaacctatcatcgcaagcatgcaagcaggaatcctcctcagaatcgagtccatggatgcaatatactcttgtaccactgggggtcgttataatcttcatcctagttttcacggcaataataatgatgaaaagacaaggtaaatcaaatcactgccagccaccagtggaagaaaaaagccttactatttatgcccaagtacagaaatcagggcctcaagagaagaaacttcatgatgccctaacagatcaggacccctgcacaaccatttatgtggctgccacagagcctgccccagagtctgtccaggaaccaaaccccaccacagtttatgccagtgtgacactgccagagagctgacccatatacccagtgaaaggactttttgaaggaggatagaagaaccaaaatccacactgaactggaccccgggtcccaagttctctgtgacagaaactgcacatctgtaaccttctccaatcagttccctggtgacggatctgcacaggcatgcttatgaagtagatgagaagtgaggcttcctgggcatgcaacctgctctgctgctgacaca

SEQ ID NO: 5 - protein, 299 aa, Saponaria officinalis
MKIYVVATIAWILLQFSAWTTTDAVTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPKSSNEANSTVRHYGPLKPTLLIT